(19) United States
(12) Patent Application Publication
(10) Pub. No.: US 2025/0188452 A1
(43) Pub. Date: Jun. 12, 2025

(54) SYSTEMS AND METHODS FOR GENETIC MODULATION TO TREAT OCULAR DISEASES

(71) Applicant: Epicrispr Biotechnologies, Inc., South San Francisco, CA (US)

(72) Inventors: Alexandra Sylvie COLLIN DE L'HORTET, South San Francisco, CA (US); Rosemarie Wenting TSOA, South San Francisco, CA (US); Alexander A. NEUBAUER, South San Francisco, CA (US); Amber Ruth SALZMAN, South San Francisco, CA (US); Yi Tang LAI, South San Francisco, CA (US)

(21) Appl. No.: 18/842,215

(22) PCT Filed: Mar. 10, 2023
(86) PCT No.: PCT/US2023/064186

§ 371 Date: Aug. 28, 2024

Related U.S. Application Data
(60) Provisional application No. 63/319,078, filed on Mar. 11, 2022.

(51) Int. Cl. C12N 15/11; C07K 14/72; C12N 9/22; C12N 15/86

(52) U.S. Cl. CPC C12N 15/111; C07K 14/723; C12N 9/22; C12N 15/86; C12N 2310/20; C12N 2750/14143

(57) ABSTRACT

Described herein are systems and methods for modulating gene expression. Also described herein are systems and methods for treating a disease or a condition by modulating gene expression.

### CROSS-REFERENCE

This application claims the benefit of U.S. Provisional Application No. 63/319,078, filed Mar. 11, 2022, which is incorporated herein by reference in its entirety.

### BACKGROUND

Aberrant expression of one or more genes (e.g., endogenous genes) can lead to a disease or a condition in a subject. In some cases, aberrant expression of an enzyme regulator (e.g., an enzyme inhibitor, such as a protease inhibitor) in a cell in the subject can lead to irregular enzymatic activity within the cell, thereby effecting various diseases. The aberrant expression can be due to one or more hereditary genetic mutations in a gene encoding the enzyme regulator. For example, mutation of Rhodopsin can lead to retinitis pigmentosa or autosomal congenital blindness.

### SUMMARY

Modifying aberrant expression of a mutant allele (e.g., a disease-causing allele) in a cell may not be sufficient to treat or cure a disease that is manifested by the aberrant expression of the mutant allele. Thus, there remains a substantial need for systems, compositions, and methods to modify the aberrant expression of the mutant allele and introduce expression of a non-disease causing allele (e.g., a wild-type allele) in a cell.

In an aspect, the present disclosure provides a system comprising: a heterologous polypeptide comprising an actuator moiety, wherein the actuator moiety is for binding an endogenous target gene encoding a target protein in a cell, to decrease expression level of the target protein, and wherein the actuator moiety substantially lacks DNA cleavage activity; a heterologous polynucleotide encoding a non-disease causing variant of the endogenous target gene that encodes the target protein; wherein the endogenous target gene is associated with an ocular disease.

In another aspect, the present disclosure provides a system comprising: a heterologous polypeptide comprising an actuator moiety, wherein the actuator moiety is for binding an endogenous target gene encoding Rhodopsin in a cell, to decrease expression level of the Rhodopsin; and a heterologous polynucleotide encoding a non-disease causing variant of the Rhodopsin, wherein the heterologous polynucleotide is not integrated into the endogenous target gene.

In another aspect, the present disclosure provides a system comprising: a heterologous nucleic acid molecule exhibiting specific binding to a target polynucleotide sequence of a chromosomal gene encoding Rhodopsin, to decrease expression level of the Rhodopsin in a cell, wherein the target polynucleotide sequence (i) is part of a non-coding region of the chromosomal gene, and (ii) exhibits at least about 70% sequence identity to the polynucleotide sequence of any one of SEQ ID NOs. 700-976.

In another aspect, the present disclosure provides one or more polynucleotides encoding the system provided heroin.

In another aspect, the present disclosure provides a method comprising administrating the system described herein to a subject in need thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates exemplary constructs encoding the dCas, the actuator moiety (effector), guide RNA (gRNA), and coding sequence (CDS) of an endogenous target gene, Rhodopsin. Legend: Promoter: photoreceptor specific or ubiquitous promoter; dCas: small dead Cas molecule such as dCasMini or equivalent effector: effector to suppress expression such as KRAB or equivalent thereof; Pr: Promoter for gRNA such as H1 or U6 or equivalent thereof; gRNA: gRNA targeting the endogenous target gene comprising Rhodopsin (RHO); Promoter 2: photoreceptor specific or ubiquitous promoter; CDS of Rhodopsin (RHO): coding sequence for the Rhodopsin gene; and Linker: P2A or equivalent thereof.

FIG. 2 illustrates a schematic for treating retinitis pigmentosa with the system described herein. AAV can be engineered to deliver an exemplary construct via subretinal or intravitreal injection to a subject in need thereof, where the expression of the exemplary construct can simultaneously decrease expression of endogenous mutated Rhodopsin and increase express of Rhodopsin encoded by the heterologous CDS of the construct.

FIG. 3 illustrates exemplary genomic loci of NM_000539 (ENST00000296271.4) and (ENSP00000296271.3) that can be targeted by the gRNA of the system and the method described herein.

FIGS. 4A-4F schematically illustrate example vectors encoding the system of the present disclosure.

FIGS. 5A and 5B schematically illustrate an example experimental procedure to assess gene modulation by the exemplary construct in isolated human retina tissues. FIG. 5C shows the normalized percent repression of wildtype Rhodopsin (RHO) expression. FIG. 5D represents the copy number of Cas transgene expression. FIG. 5E represents the copy number of exogenous RHO expression.

FIG. 6A schematically illustrate an example experimental procedure to assess gene modulation by the exemplary construct in 3D retinal organoids. FIG. 6B represents the percent reduction of wildtype RHO expression. FIG. 6C shows the copy number of exogenous RHO expression.

FIGS. 7A and 7B represent the immunostaining of human retina tissue transduced with the exemplary construct for endogenous and exogenous RHO (FIG. 7A) and dCas (FIG. 7B). FIG. 7C shows immunostaining of 3D retinal organoids of wild-type and p23H RHO mutant.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [aS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “sequence identity” generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 95%, or at least 98% sequence identity with any sequence provided herein.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence). For example, a gene can refer to a portion of the gene that is near or adjacent to a transcription start site (TSS) of the gene. The gene (e.g., that is targeted as disclosed herein) can be at least or up to about 2,000 nucleobases, at least or up to about 1,800 nucleobases, at least or up to about 1,600 nucleobases, at least or up to about 1,500 nucleobases, at least or up to about 1,400 nucleobases, at least or up to about 1,200 nucleobases, at least or up to about 1,000 nucleobases, at least or up to about 900 nucleobases, at least or up to about 800 nucleobases, at least or up to about 700 nucleobases, at least or up to about 600 nucleobases, at least or up to about 500 nucleobases, at least or up to about 400 nucleobases, at least or up to about 300 nucleobases, at least or up to about 200 nucleobases, at least or up to about 100 nucleobases, or at least or up to about 50 nucleobases away from the TSS of the gene.

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “expression profile” generally refers to quantitative (e.g., abundance) and qualitative expression of one or more genes in a sample (e.g., a cell). The one or more genes can be expressed and ascertained in the form of a nucleic acid molecule (e.g., an mRNA or other RNA transcript). Alternatively or in addition to, the one or more genes can be expressed and ascertained in the form of a polypeptide (e.g., a protein measured via Western blot). An expression profile of a gene may be defined as a shape of an expression level of the gene over a time period (e.g., at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 16 hours, at least or up to about 18 hours, at least or up to about 24 hours, at least or up to about 36 hours, at least or up to about 48 hours, at least up to about 3 days, at least up to about 4 days, at least up to about 5 days, at least up to about 6 days, at least up to about 7 days, at least up to about 8 days, at least up to about 9 days, at least up to about 10 days, at least up to about 11 days, at least up to about 12 days, at least up to about 13 days, at least up to about 14 days, etc.). Alternatively, an expression profile of a gene may be defined as an expression level of the gene at a time point of interest (e.g., the expression level of the gene measured at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 16 hours, at least or up to about 18 hours, at least or up to about 24 hours, at least or up to about 36 hours, at least or up to about 48 hours, at least up to about 3 days, at least up to about 4 days, at least up to about 5 days, at least up to about 6 days, at least up to about 7 days, at least up to about 8 days, at least up to about 9 days, at least up to about 10 days, at least up to about 11 days, at least up to about 12 days, at least up to about 13 days, or at least up to about 14 days after treating a cell to induce such expression level.)

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety.

The terms “engineered” and “modified” are used interchangeably herein. The terms “engineering” and “modifying” are used interchangeably herein. The terms “engineered cell” or “modified cell” are used interchangeably herein. The terms “engineered characteristic” and “modified characteristic” are used interchangeably herein.

The term “enhanced expression,” “increased expression,” or “upregulated expression” generally refers to production of a moiety of interest (e.g., a polynucleotide or a polypeptide) to a level that is above a normal level of expression of the moiety of interest in a host strain (e.g., a host cell). The normal level of expression can be substantially zero (or null) or higher than zero. The moiety of interest can comprise an endogenous gene or polypeptide construct of the host strain. The moiety of interest can comprise a heterologous gene or polypeptide construct that is introduced to or into the host strain. For example, a heterologous gene encoding a polypeptide of interest can be knocked-in (KI) to a genome of the host strain for enhanced expression of the polypeptide of interest in the host strain.

The term “enhanced activity,” “increased activity,” or “upregulated activity” generally refers to activity of a moiety of interest (e.g., a polynucleotide or a polypeptide) that is modified to a level that is above a normal level of activity of the moiety of interest in a host strain (e.g., a host cell). The normal level of activity can be substantially zero (or null) or higher than zero. The moiety of interest can comprise a polypeptide construct of the host strain. The moiety of interest can comprise a heterologous polypeptide construct that is introduced to or into the host strain. For example, a heterologous gene encoding a polypeptide of interest can be knocked-in (KI) to a genome of the host strain for enhanced activity of the polypeptide of interest in the host strain.

The term “reduced expression,” “decreased expression,” or “downregulated expression” generally refers to a production of a moiety of interest (e.g., a polynucleotide or a polypeptide) to a level that is below a normal level of expression of the moiety of interest in a host strain (e.g., a host cell). The normal level of expression is higher than zero. The moiety of interest can comprise an endogenous gene or polypeptide construct of the host strain. In some cases, the moiety of interest can be knocked-out or knocked-down in the host strain. In some examples, reduced expression of the moiety of interest can include a complete inhibition of such expression in the host strain.

The term “reduced activity,” “decreased activity,” or “downregulated activity” generally refers to activity of a moiety of interest (e.g., a polynucleotide or a polypeptide) that is modified to a level that is below a normal level of activity of the moiety of interest in a host strain (e.g., a host cell). The normal level of activity is higher than zero. The moiety of interest can comprise an endogenous gene or polypeptide construct of the host strain. In some cases, the moiety of interest can be knocked-out or knocked-down in the host strain. In some examples, reduced activity of the moiety of interest can include a complete inhibition of such activity in the host strain.

The term “subject,” “individual,” or “patient,” as used interchangeably herein, generally refers to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The term “treatment” or “treating” generally refers to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” generally refers to the quantity of a composition, for example a composition comprising heterologous polypeptides, heterologous polynucleotides, and/or modified cells (e.g., modified stem cells), that is sufficient to result in a desired activity upon administration to a subject in need thereof. Within the context of the present disclosure, the term “therapeutically effective” generally refers to that quantity of a composition that is sufficient to delay the manifestation, arrest the progression, relieve or alleviate at least one symptom of a disorder treated by the methods of the present disclosure.

### Overview

Gene expression underpins various physiological and pathological effects in cells and tissues, contributing to many diseases and conditions, thus agents that modulate expression of specific genes in a desirable way could have therapeutic benefit.

Developing agents that elicit robust, persistent, and/or reversible changes in gene expression has proven challenging, however, as many candidate therapeutics achieve only modest or short lived effects, or conversely result in off-target effects. Additionally, many current approaches to gene editing and genome engineering can result in off-target effects that can be associated with undesirable toxicity profiles, and in some cases, undesirable effects can be permanent. There is thus a need for novel strategies to regulate gene expression that allow robust, persistent, and/or reversible modulation of target gene expression and activity, for example, expression of genes that impact human disease.

For instance, ribonucleic acid interference (RNAi) can be used to silence aberrant expression of a mutant allele (e.g., a disease-causing allele) by generating knockdowns at the messenger RNA (mRNA) level in a sequence specific manner. However, by only interfering at the mRNA level and not at the upstream genetic level (e.g., chromosomal level), the effect of RNAi can be limited because more mRNAs can be continuously generated from the mutant allele in a cell. Thus, there remains a need for an alternative strategy that can act on the chromosomal level to regulate the aberrant expression of the mutant allele. Therefore, some aspects of the present disclosure provide systems, compositions, and methods for regulating expression level of a mutant allele of a gene in a cell via directly interacting with the mutant allele at the chromosomal level, e.g., via using endonuclease such as CRISPR (Clustered Regularly Interspaced Short Palindromic Repeats)/Cas (CRISPR-associated) system.

Furthermore, regulating the aberrant expression of a mutant allele of a gene in a cell may not be sufficient to treat or ameliorate a condition (e.g., a disease) of a subject. Thus, there remains a need for an alternative strategy that not only targets the aberrant expression of the mutant allele, but also introduce expression of a non-disease causing allele (e.g., a wild-type allele) of the gene to the subject. Therefore, some aspects of the present disclosure provides systems, compositions, and methods for achieve both (i) regulation of a disease causing allele of a gene and (ii) introduction of a non-disease causing allege of the gene to a cell or a subject.

### Systems, Compositions, and Methods Thereof

In an aspect, the present disclosure provides a system comprising a heterologous polypeptide comprising an actuator moiety, wherein the actuator moiety is for binding an endogenous target gene encoding a target protein in a cell, to decrease expression level of the target protein, and wherein the actuator moiety substantially lacks DNA cleavage activity; and a heterologous polynucleotide encoding a non-disease causing variant of the endogenous target gene that encodes the target gene, wherein the endogenous target gene is associated with an ocular disease. In an aspect, the present disclosure provides one or more polynucleotides encoding the heterologous polynucleotide described herein. In an aspect, the present disclosure provides a method comprising decreasing expression level of an endogenous target gene encoding a target protein in a cell, via action of a heterologous polypeptide comprising an actuator moiety, wherein the actuator moiety is for binding the endogenous target gene, and wherein the actuator moiety substantially lacks DNA cleavage activity; and contacting the cell with a heterologous polynucleotide encoding a non-disease causing variant of the endogenous target gene, wherein the endogenous target gene is associated with an ocular disease. In some embodiments, the endogenous target gene comprises a disease causing allele of the target protein. In some embodiments, the endogenous target gene comprises a disease causing allele of the target protein, where the disease causing allele is a mutant allele. In some embodiments, the endogenous target gene comprises a non-disease causing allele of the target protein. In some embodiments, the endogenous target gene comprises a non-disease causing allele of the target protein, where the non-disease causing allele is a wild type allele. In some embodiments, heterologous polynucleotide encoding the non-disease causing variant is codon optimized for expression in mammalian cell (e.g., human cell). In some embodiments, the non-disease causing variant is an engineered variant of a wild type allele.

In an aspect, the present disclosure provides a system comprising: a heterologous polypeptide comprising an actuator moiety, wherein the actuator moiety is for binding an endogenous target gene encoding a G-protein-coupled receptor (GPCR) in a cell, to decrease expression level of the GPCR; and a heterologous polynucleotide encoding a non-disease causing variant of the GPCR. In some embodiments, the GPCR comprises an opsin. In some embodiments, the opsin can include L-cone (red-cone) opsin, M-cone (green-cone) opsin, S-cone (blue-cone) opsin, Rhodopsin, Encephalopsin, panopsin, Melanopsin, Neuropsin, Peropsin, or Retinal G protein coupled receptor. In some embodiments, the opsin is Rhodopsin. In an aspect, the present disclosure provides a system comprising: a heterologous polypeptide comprising an actuator moiety, wherein the actuator moiety is for binding an endogenous target gene encoding a Rhodopsin in a cell, to decrease expression level of the Rhodopsin; and a heterologous polynucleotide encoding a non-disease causing variant of the Rhodopsin.

In some embodiments, described herein is a system for modulating a gene expression of an endogenous target gene described herein. In some embodiments, the system comprises a heterologous polypeptide comprising an actuator moiety, wherein the actuator moiety is for binding an endogenous target gene encoding a target protein in a cell, to decrease expression level of the target protein, and wherein the actuator moiety substantially lacks DNA cleavage activity; and a heterologous polynucleotide encoding a non-disease causing variant of the endogenous target gene. In some embodiments, the endogenous target gene is a non-disease causing variant. In some embodiments, the non-disease causing variant is a wild type variant. In some embodiments, the endogenous target gene is a disease causing variant. In some embodiments, the system comprises the heterologous polynucleotide not integrated into the endogenous target gene. In some embodiments, the system comprises the heterologous polypeptide that is under the control of a tissue-specific promoter. For example, the tissue-specific promoter can be a rod cell specific promoter, a cone cell specific promoter, a retina cell specific promoter, a photoreceptor specific promoter, or a combination thereof. In some embodiments, the tissue-specific promoter is a photoreceptor specific promoter. In some embodiments, the tissue-specific promoter is a Rhodopsin promoter. Non-limiting example pf tissue-specific promoter can include MOPS promoter, GRK1 promoter, IRBP promoter, PR2.1 promoter, IRBP/GNAT2 promoter, VMD2 promoter, VEcad/VEcadherin promoter, or a combination thereof.

In some embodiments, the system comprises the heterologous polypeptide that is under the control of a constitutive promoter. Non-limiting example of constitutive promoter can include CMV promoter, EF1a promoter, CAG promoter, PGK promoter, TRE promoter, U6 promoter, or UAS promoter. For example, the constitutive promoter can be a Pol III promoter (e.g., 7SK, U6, H1, etc.). In another example, the constitutive promoter can be a Pol II promoter (e.g., CMV, RSV, etc.). In some embodiments, the actuator moiety comprises a nuclease such as an endonuclease (e.g., a heterologous endonuclease). In some embodiments, the nuclease can be a deactivated nuclease such as a deactivated endonuclease, where the deactivated endonuclease does not cleave nucleic acid.

In some embodiments, the system comprises a guide nucleic acid. In some embodiments, a guide nucleic acid capable of forming a complex with the actuator moiety, wherein the complex binds the endogenous target gene. In some embodiments, the guide nucleic acid comprises a plurality of different guide nucleic acids capable of targeting different regions of the endogenous target gene.

In some embodiments, the system comprises an actuator moiety or a heterologous polynucleotide encoding the actuator moiety. In some embodiments, the actuator moiety is coupled to a transcriptional repressor. In some embodiments, the actuator moiety is fused to the transcriptional repressor.

In some embodiments, the system modulates a gene expression of an endogenous target gene in a cell. In some embodiments, the cell is an eye cell such as a rod cell, a cone cell, or a retina cell. In some embodiments, the cell is a photoreceptor cell, a bipolar cell, a retinal ganglion cell, a horizontal cell, or an amacrine cells. In some embodiments, the cell is a cell of pigmented layer. In some embodiments, the cell is a cell of layer of rods and cones. In some embodiments, the cell is a cell of membrana limitans externa. In some embodiments, the cell is a cell of outer nuclear layer. In some embodiments, the cell is a cell of outer plexiform layer. In some embodiments, the cell is a cell of inner nuclear layer. In some embodiments, the cell is a cell of inner plexiform layer. In some embodiments, the cell is a cell of ganglionic layer. In some embodiments, the cell is a cell of stratum opticum. In some embodiments, the cell is a cell of membrana limitans interna. In some embodiments, the cell a retinal pigment epithelium (RPE) cell. In some embodiments, the cell is in a retina tissue. In some embodiments, the cell is in a human retina tissue.

In some embodiments, described herein is one or more polynucleotides encoding the system described herein. In some embodiments, the one or more polynucleotides comprise a single polynucleotide encoding at least the heterologous polypeptide and the heterologous polynucleotide. In some embodiments, the single polynucleotide further encodes the guide nucleic acid. In some embodiments, the single polynucleotide has a size of less than or equal to about 5 kilobases (kb). In some embodiments, the single polynucleotide has a size of less than or equal to about 4.7 kilobases. In some embodiments, the single polynucleotide has a size of less than or equal to about 0.1 kb to about 10 kb. In some embodiments, the single polynucleotide has a size of less than or equal to about 10 kb to about 9 kb, about 10 kb to about 8 kb, about 10 kb to about 7 kb, about 10 kb to about 6 kb, about 10 kb to about 5 kb, about 10 kb to about 4.7 kb, about 10 kb to about 4 kb, about 10 kb to about 3 kb, about 10 kb to about 2 kb, about 10 kb to about 1 kb, about 10 kb to about 0.1 kb, about 9 kb to about 8 kb, about 9 kb to about 7 kb, about 9 kb to about 6 kb, about 9 kb to about 5 kb, about 9 kb to about 4.7 kb, about 9 kb to about 4 kb, about 9 kb to about 3 kb, about 9 kb to about 2 kb, about 9 kb to about 1 kb, about 9 kb to about 0.1 kb, about 8 kb to about 7 kb, about 8 kb to about 6 kb, about 8 kb to about 5 kb, about 8 kb to about 4.7 kb, about 8 kb to about 4 kb, about 8 kb to about 3 kb, about 8 kb to about 2 kb, about 8 kb to about 1 kb, about 8 kb to about 0.1 kb, about 7 kb to about 6 kb, about 7 kb to about 5 kb, about 7 kb to about 4.7 kb, about 7 kb to about 4 kb, about 7 kb to about 3 kb, about 7 kb to about 2 kb, about 7 kb to about 1 kb, about 7 kb to about 0.1 kb, about 6 kb to about 5 kb, about 6 kb to about 4.7 kb, about 6 kb to about 4 kb, about 6 kb to about 3 kb, about 6 kb to about 2 kb, about 6 kb to about 1 kb, about 6 kb to about 0.1 kb, about 5 kb to about 4.7 kb, about 5 kb to about 4 kb, about 5 kb to about 3 kb, about 5 kb to about 2 kb, about 5 kb to about 1 kb, about 5 kb to about 0.1 kb, about 4.7 kb to about 4 kb, about 4.7 kb to about 3 kb, about 4.7 kb to about 2 kb, about 4.7 kb to about 1 kb, about 4.7 kb to about 0.1 kb, about 4 kb to about 3 kb, about 4 kb to about 2 kb, about 4 kb to about 1 kb, about 4 kb to about 0.1 kb, about 3 kb to about 2 kb, about 3 kb to about 1 kb, about 3 kb to about 0.1 kb, about 2 kb to about 1 kb, about 2 kb to about 0.1 kb, or about 1 kb to about 0.1 kb. In some embodiments, the single polynucleotide has a size of less than or equal to about 10 kb, about 9 kb, about 8 kb, about 7 kb, about 6 kb, about 5 kb, about 4.7 kb, about 4 kb, about 3 kb, about 2 kb, about 1 kb, or about 0.1 kb. In some embodiments, the single polynucleotide has a size of less than or equal to at least about 10 kb, about 9 kb, about 8 kb, about 7 kb, about 6 kb, about 5 kb, about 4.7 kb, about 4 kb, about 3 kb, about 2 kb, or about 1 kb. In some embodiments, the single polynucleotide has a size of less than or equal to at most about 9 kb, about 8 kb, about 7 kb, about 6 kb, about 5 kb, about 4.7 kb, about 4 kb, about 3 kb, about 2 kb, about 1 kb, or about 0.1 kb.

In some embodiments, the system decreases the expression of the endogenous target gene (e.g., a disease causing allele or non-disease causing allele of GPCR described herein or Rhodopsin) encoding the target protein by at least about 0.01 fold to about 5,000 fold (e.g., as compared to a control cell lacking the heterologous polypeptide and/or the guide nucleic acid). In some embodiments, the system decreases the expression of the endogenous target gene encoding the target protein by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the system decreases the expression of the endogenous target gene encoding the target protein by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the system decreases the expression of the endogenous target gene encoding the target protein by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the system decreases the expression of the endogenous target gene encoding the target protein by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the system decreases the expression of endogenous Rhodopsin by at least about 0.01 fold to about 5,000 fold (e.g., as compared to a control cell lacking the heterologous polypeptide and/or the guide nucleic acid). In some embodiments, the system decreases the expression of Rhodopsin by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the system decreases the expression of Rhodopsin by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the system decreases the expression of Rhodopsin by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the system decreases the expression of Rhodopsin by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the system increases the expression of the non-disease causing variant of the target gene encoding the target protein by at least about 0.01 fold to about 5,000 fold (e.g., as compared to a control cell lacking the heterologous polypeptide and/or the guide nucleic acid). In some embodiments, the system increases the expression of the non-disease causing variant of the target gene encoding the target protein by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the system increases the expression of the non-disease causing variant of the target gene encoding the target protein by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the system increases the expression of the non-disease causing variant of the target gene encoding the target protein by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the system increases the expression of the non-disease causing variant of the target gene encoding the target protein by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the system increases the expression of the non-disease causing variant of the target gene (e.g., GPCR described herein or Rhodopsin) encoding the target protein, where the target gene comprises GPCR. In some embodiments, the system increases the expression of non-disease causing variant of GPCR by at least about 0.01 fold to about 5,000 fold (e.g., as compared to a control cell lacking the heterologous polypeptide and/or the guide nucleic acid). In some embodiments, the system increases the expression of non-disease causing variant of GPCR by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the system increases the expression of non-disease causing variant of GPCR by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the system increases the expression of non-disease causing variant of GPCR by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the system increases the expression of non-disease causing variant of GPCR by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the system increases the expression of non-disease causing variant of Rhodopsin by at least about 0.01 fold to about 5,000 fold (e.g., as compared to a control cell lacking the heterologous polypeptide and/or the guide nucleic acid). In some embodiments, the system increases the expression of non-disease causing variant of Rhodopsin by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the system increases the expression of non-disease causing variant of Rhodopsin by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the system increases the expression of non-disease causing variant of Rhodopsin by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the system increases the expression of non-disease causing variant of Rhodopsin by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the system decreases apoptosis propensity of the cell expressing the endogenous target gene. In some embodiments, the system decreases apoptosis propensity of the cell expressing the endogenous target gene by decreasing expression level of the target protein, expressing the non-disease causing variant of the target protein, or a combination thereof. In some embodiments, the endogenous target protein comprises a GPCR described herein. In some embodiments, the endogenous target protein comprises Rhodopsin. Apoptosis propensity of the cell can be ascertained by measuring apoptosis of a population of the cells, or by measuring a degree of apoptosis markers such as Annexin, Caspase, DNA fragmentation (e.g., TUNEL assay), Cytochrome C release, or Glutathione. In some embodiments, the system decreases apoptosis propensity of the cell expressing the endogenous target gene by at least about 0.01 fold to about 5,000 fold (e.g., as compared to a control cell lacking the heterologous polypeptide and/or the guide nucleic acid). In some embodiments, the system decreases apoptosis propensity of the cell expressing the endogenous target gene by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the system decreases apoptosis propensity of the cell expressing the endogenous target gene by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the system decreases apoptosis propensity of the cell expressing the endogenous target gene by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the system decreases apoptosis propensity of the cell expressing the endogenous target gene by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the system decreases endoplasmic reticulum (ER) stress of the cell expressing the endogenous target gene. In some embodiments, the system decreases endoplasmic reticulum (ER) stress of the cell expressing the endogenous target gene by decreasing expression level of the target protein, expressing the non-disease causing variant of the target protein, or a combination thereof. In some embodiments, the endogenous target protein comprises a GPCR described herein. In some embodiments, the endogenous target protein comprises Rhodopsin. ER stress can be ascertained by measuring the activation or upregulation of various components of the endogenous unfolded protein response (UPR) in the cell under normal condition or under cellular stress condition. In some embodiments, the system decreases endoplasmic reticulum (ER) stress of the cell expressing the endogenous target gene by at least about 0.01 fold to about 5,000 fold (e.g., as compared to a control cell lacking the heterologous polypeptide and/or the guide nucleic acid). In some embodiments, the system decreases endoplasmic reticulum (ER) stress of the cell expressing the endogenous target gene by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the system decreases endoplasmic reticulum (ER) stress of the cell expressing the endogenous target gene by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the system decreases endoplasmic reticulum (ER) stress of the cell expressing the endogenous target gene by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the system decreases endoplasmic reticulum (ER) stress of the cell expressing the endogenous target gene by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the system comprises a guide nucleic acid or one or more polynucleotides encoding a guide nucleic acid, where the guide nucleic acid targets an endogenous target gene described herein. In some embodiments, the guide nucleic acid can be complexed with an actuator moiety described herein. In some embodiments the guide nucleic acid can direct the actuator moiety to the endogenous target gene in the cell.

In some embodiments, described herein is a composition comprising any component or any combination of components of the system described herein. In some embodiments, the composition comprises at least one of the heterologous polypeptide described herein. In some embodiments, the compositions comprises at least one of the heterologous polynucleotide described herein. In some embodiments, the composition comprises at least one of the heterologous polypeptide described herein and at least one of the heterologous polynucleotide described herein. In some embodiments, the composition can be further formulated into a pharmaceutical composition. For example, the composition can comprise at least one pharmaceutically acceptable carrier.

Described herein, in some aspects, is a method comprising: decreasing expression level of an endogenous target gene encoding a target protein in a cell, via action of a heterologous polypeptide comprising an actuator moiety, wherein the actuator moiety is for binding the endogenous target gene, and wherein the actuator moiety substantially lacks DNA cleavage activity; and contacting the cell with a heterologous polynucleotide encoding a non-disease causing variant of the endogenous target gene. In some embodiments, the method comprises determining that the subject has certain condition. In some embodiments, the method comprises selecting for the subject to be treated by the method and the system described herein by determining if the subject harbors a mutant allele or a disease-causing allele of the endogenous target gene. In some embodiments, once the subject is determined to harbor the mutant allele or the disease-causing allele of the endogenous target gene, a system described herein, any component of the system described herein, or any combination of the component of the system described herein can be administered to the subject to treat the disease or condition. In some embodiments, the endogenous target gene comprises a disease causing allele of the target protein. In some embodiments, the endogenous target gene comprises a non-disease causing allele of the endogenous target protein. In some embodiments, the non-disease causing allele is a wild type allele. In some embodiments, the endogenous target gene is a GPCR. In some embodiments, the GPCR comprises an opsin. In some embodiments, the opsin can include L-cone (red-cone) opsin, M-cone (green-cone) opsin, S-cone (blue-cone) opsin, Rhodopsin, Encephalopsin, panopsin, Melanopsin, Neuropsin, Peropsin, or Retinal G protein coupled receptor. In some embodiments, the opsin is Rhodopsin. In some embodiments, the endogenous target gene is Rhodopsin. In some embodiments, described herein is a method for administering the system descried herein to a subject in need thereof. In some embodiments, the method comprises determining whether the subject has or is suspected of having retinitis pigmentosa 4 (RP4).

In some embodiments, the method decreases the expression of the endogenous target gene encoding the target protein by at least about 0.01 fold to about 5,000 fold (e.g., as compared to a control cell lacking the heterologous polypeptide and/or the guide nucleic acid). In some embodiments, the method decreases the expression of the endogenous target gene encoding the target protein by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the method decreases the expression of the endogenous target gene encoding the target protein by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method decreases the expression of the endogenous target gene encoding the target protein by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the method decreases the expression of the endogenous target gene encoding the target protein by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the method decreases the expression of the endogenous target gene encoding the target protein, where the target protein is a GPCR described or Rhodopsin. In some embodiments, the method decreases the expression of GPCR by at least about 0.01 fold to about 5,000 fold (e.g., as compared to a control cell lacking the heterologous polypeptide and/or the guide nucleic acid). In some embodiments, the method decreases the expression of GPCR by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the method decreases the expression of GPCR by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method decreases the expression of GPCR by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the method decreases the expression of GPCR by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the method decreases the expression of endogenous Rhodopsin by at least about 0.01 fold to about 5,000 fold (e.g., as compared to a control cell lacking the heterologous polypeptide and/or the guide nucleic acid). In some embodiments, the method decreases the expression of Rhodopsin by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the method decreases the expression of Rhodopsin by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method decreases the expression of Rhodopsin by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the method decreases the expression of Rhodopsin by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method decreases apoptosis propensity of the cell expressing the endogenous target gene. In some embodiments, the method decreases apoptosis propensity of the cell expressing the endogenous target gene by decreasing expression level of the target protein, expressing the non-disease causing variant of the target protein, or a combination thereof. In some embodiments, the target protein comprises a GPCR described herein. In some embodiments, the target protein comprises Rhodopsin. Apoptosis propensity of the cell can be ascertained by measuring apoptosis of a population of the cells, or by measuring a degree of apoptosis markers such as Annexin (e.g., by propidium iodide staining), Caspase, DNA fragmentation (e.g., TUNEL assay), Cytochrome C release, or Glutathione. In some embodiments, the method decreases apoptosis propensity of the cell expressing the endogenous target gene by at least about 0.01 fold to about 5,000 fold (e.g., as compared to a control cell lacking the heterologous polypeptide and/or the guide nucleic acid). In some embodiments, the method decreases apoptosis propensity of the cell expressing the endogenous target gene by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the method decreases apoptosis propensity of the cell expressing the endogenous target gene by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method decreases apoptosis propensity of the cell expressing the endogenous target gene by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the method decreases apoptosis propensity of the cell expressing the endogenous target gene by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the method increases the expression of the non-disease causing variant of the target gene encoding the target protein by at least about 0.01 fold to about 5,000 fold (e.g., as compared to a control cell lacking the heterologous polypeptide and/or the guide nucleic acid). In some embodiments, the method increases the expression of the non-disease causing variant of the target gene encoding the target protein by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the method increases the expression of the non-disease causing variant of the target gene encoding the target protein by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method increases the expression of the non-disease causing variant of the target gene encoding the target protein by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the method increases the expression of the non-disease causing variant of the target gene encoding the target protein by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the method increases the expression of the non-disease causing variant of the target gene (e.g., GPCR described herein or Rhodopsin) encoding the target protein, where the target gene comprises GPCR. In some embodiments, the method increases the expression of non-disease causing variant of GPCR by at least about 0.01 fold to about 5,000 fold (e.g., as compared to a control cell lacking the heterologous polypeptide and/or the guide nucleic acid). In some embodiments, the method increases the expression of non-disease causing variant of GPCR by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the method increases the expression of non-disease causing variant of GPCR by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method increases the expression of non-disease causing variant of GPCR by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the method increases the expression of non-disease causing variant of GPCR by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the method increases the expression of non-disease causing variant of Rhodopsin by at least about 0.01 fold to about 5,000 fold (e.g., as compared to a control cell lacking the heterologous polypeptide and/or the guide nucleic acid). In some embodiments, the method increases the expression of non-disease causing variant of Rhodopsin by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the method increases the expression of non-disease causing variant of Rhodopsin by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method increases the expression of non-disease causing variant of Rhodopsin by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the method increases the expression of non-disease causing variant of Rhodopsin by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the method decreases endoplasmic reticulum (ER) stress of the cell expressing the endogenous target gene. In some embodiments, the method decreases endoplasmic reticulum (ER) stress of the cell expressing the endogenous target gene by decreasing expression level of the target protein, expressing the non-disease causing variant of the target protein, or a combination thereof. In some embodiments, the endogenous target protein comprises a GPCR described herein. In some embodiments, the endogenous target protein comprises Rhodopsin. ER stress can be ascertained by measuring the activation or upregulation of various components of the endogenous unfolded protein response (UPR) in the cell under normal condition or under cellular stress condition. In some embodiments, the method decreases endoplasmic reticulum (ER) stress of the cell expressing the endogenous target gene by at least about 0.01 fold to about 5,000 fold (e.g., as compared to a control cell lacking the heterologous polypeptide and/or the guide nucleic acid). In some embodiments, the method decreases endoplasmic reticulum (ER) stress of the cell expressing the endogenous target gene by at least about 0.01 fold to about 0.05 fold, about 0.01 fold to about 0.1 fold, about 0.01 fold to about 0.5 fold, about 0.01 fold to about 1 fold, about 0.01 fold to about 5 fold, about 0.01 fold to about 10 fold, about 0.01 fold to about 50 fold, about 0.01 fold to about 100 fold, about 0.01 fold to about 500 fold, about 0.01 fold to about 1,000 fold, about 0.01 fold to about 5,000 fold, about 0.05 fold to about 0.1 fold, about 0.05 fold to about 0.5 fold, about 0.05 fold to about 1 fold, about 0.05 fold to about 5 fold, about 0.05 fold to about 10 fold, about 0.05 fold to about 50 fold, about 0.05 fold to about 100 fold, about 0.05 fold to about 500 fold, about 0.05 fold to about 1,000 fold, about 0.05 fold to about 5,000 fold, about 0.1 fold to about 0.5 fold, about 0.1 fold to about 1 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 5,000 fold, about 0.5 fold to about 1 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 5,000 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 5,000 fold, about 5 fold to about 10 fold, about 5 fold to about 50 fold, about 5 fold to about 100 fold, about 5 fold to about 500 fold, about 5 fold to about 1,000 fold, about 5 fold to about 5,000 fold, about 10 fold to about 50 fold, about 10 fold to about 100 fold, about 10 fold to about 500 fold, about 10 fold to about 1,000 fold, about 10 fold to about 5,000 fold, about 50 fold to about 100 fold, about 50 fold to about 500 fold, about 50 fold to about 1,000 fold, about 50 fold to about 5,000 fold, about 100 fold to about 500 fold, about 100 fold to about 1,000 fold, about 100 fold to about 5,000 fold, about 500 fold to about 1,000 fold, about 500 fold to about 5,000 fold, or about 1,000 fold to about 5,000 fold (e.g., as compared to a control cell lacking the heterologous polypeptide and/or the guide nucleic acid). In some embodiments, the method decreases endoplasmic reticulum (ER) stress of the cell expressing the endogenous target gene by at least about 0.01 fold, about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid). In some embodiments, the method decreases endoplasmic reticulum (ER) stress of the cell expressing the endogenous target gene by at least at least about 0.01 fold, about 0.05 fold, about 0.1 fold, about 0.5 fold, about 1 fold, about 5 fold, about 10 fold, about 50 fold, about 100 fold, about 500 fold, or about 1,000 fold (e.g., as compared to a control cell lacking the heterologous polypeptide and/or the guide nucleic acid). In some embodiments, the method decreases endoplasmic reticulum (ER) stress of the cell expressing the endogenous target gene by at least at most about 0.05 fold, about 0.1 fold, about 0.5 fold, about 1 fold, about 5 fold, about 10 fold, about 50 fold, about 100 fold, about 500 fold, about 1,000 fold, or about 5,000 fold (e.g., as compared to a control cell lacking the heterologous polypeptide and/or the guide nucleic acid).

In some embodiments, the systems and methods described herein can yield a copy number of the non-disease causing variant of the endogenous target gene (e.g., Rhodopsin) of at least about 10,000 to about 1,500,000. In some embodiments, the systems and methods described herein can yield a copy number of the non-disease causing variant of the endogenous target gene (e.g., Rhodopsin) of at least about 10,000 to about 25,000, at least about 10,000 to about 50,000, at least about 10,000 to about 75,000, at least about 10,000 to about 100,000, at least about 10,000 to about 250,000, at least about 10,000 to about 500,000, at least about 10,000 to about 750,000, at least about 10,000 to about 1,000,000, at least about 10,000 to about 1,250,000, at least about 10,000 to about 1,500,000, at least about 25,000 to about 50,000, at least about 25,000 to about 75,000, at least about 25,000 to about 100,000, at least about 25,000 to about 250,000, at least about 25,000 to about 500,000, at least about 25,000 to about 750,000, at least about 25,000 to about 1,000,000, at least about 25,000 to about 1,250,000, at least about 25,000 to about 1,500,000, at least about 50,000 to about 75,000, at least about 50,000 to about 100,000, at least about 50,000 to about 250,000, at least about 50,000 to about 500,000, at least about 50,000 to about 750,000, at least about 50,000 to about 1,000,000, at least about 50,000 to about 1,250,000, at least about 50,000 to about 1,500,000, at least about 75,000 to about 100,000, at least about 75,000 to about 250,000, at least about 75,000 to about 500,000, at least about 75,000 to about 750,000, at least about 75,000 to about 1,000,000, at least about 75,000 to about 1,250,000, at least about 75,000 to about 1,500,000, at least about 100,000 to about 250,000, at least about 100,000 to about 500,000, at least about 100,000 to about 750,000, at least about 100,000 to about 1,000,000, at least about 100,000 to about 1,250,000, at least about 100,000 to about 1,500,000, at least about 250,000 to about 500,000, at least about 250,000 to about 750,000, at least about 250,000 to about 1,000,000, at least about 250,000 to about 1,250,000, at least about 250,000 to about 1,500,000, at least about 500,000 to about 750,000, at least about 500,000 to about 1,000,000, at least about 500,000 to about 1,250,000, at least about 500,000 to about 1,500,000, at least about 750,000 to about 1,000,000, at least about 750,000 to about 1,250,000, at least about 750,000 to about 1,500,000, at least about 1,000,000 to about 1,250,000, at least about 1,000,000 to about 1,500,000, or about 1,250,000 to about 1,500,000. In some embodiments, the systems and methods described herein can yield a copy number of the non-disease causing variant of the endogenous target gene (e.g., Rhodopsin) of about10,000, about 25,000, about 50,000, about 75,000, about 100,000, about 250,000, about 500,000, about 750,000, about 1,000,000, about 1,250,000, or about 1,500,000. In some embodiments, the systems and methods described herein can yield a copy number of the non-disease causing variant of the endogenous target gene (e.g., Rhodopsin) of at most about 25,000, about 50,000, about 75,000, about 100,000, about 250,000, about 500,000, about 750,000, about 1,000,000, about 1,250,000, or about 1,500,000.

### Heterologous Polypeptide Comprising an Actuator Moiety

In various aspects of the present disclosure, the heterologous polypeptide comprising the actuator moiety, as disclosed herein, can be utilized for binding a target gene, such as an endogenous target gene (e.g., a chromosomal DNA sequence). The actuator moiety can be a nuclease, such as an endonuclease (e.g., a heterologous endonuclease). Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof; any variant thereof and any fragment thereof.

In some embodiments, the actuator moiety can comprise a DNA nuclease such as an engineered (e.g., programmable or targetable) DNA nuclease that is nuclease-deficient. In some embodiments, the actuator moiety can comprise a nuclease-null DNA binding protein derived from a DNA nuclease that does not induce transcriptional activation or repression of a target DNA sequence unless it is present in a complex with one or more heterologous gene effectors of the disclosure. In some embodiments, the actuator moiety can comprise a nuclease-null DNA binding protein derived from a DNA nuclease that can induce transcriptional activation or repression of a target DNA sequence (e.g., which can be altered or augmented by the presence of a heterologous gene effector of the disclosure).

In some embodiments, the actuator moiety can comprise an RNA nuclease such as an engineered (e.g., programmable or targetable) RNA nuclease. In some embodiments, the actuator moiety can comprise a nuclease-null RNA binding protein derived from an RNA nuclease that does not induce transcriptional activation or repression of a target RNA sequence unless it is present in a complex with one or more heterologous gene effectors of the disclosure. In some embodiments, the actuator moiety can comprise a nuclease-null RNA binding protein derived from a RNA nuclease that can induce transcriptional activation or repression of a target RNA sequence (e.g., which can be altered or augmented by the presence of a heterologous gene effector of the disclosure).

In some embodiments, the actuator moiety can comprise a nucleic acid-guided targeting system. In some embodiments, the actuator moiety can comprise a DNA-guided targeting system. In some embodiments, the actuator moiety can comprise an RNA-guided targeting system. The nucleic acid-guided targeting system can comprise and utilize, for example, a guide nucleic acid sequence that facilitates specific binding of a CRISPR-Cas system (e.g., a nuclease deficient form thereof, such as dCas9 or dCas14) to a target gene (e.g., target endogenous gene) or target gene regulatory sequence. Binding specificity can be determined by use of a guide nucleic acid, such as a single guide RNA (sgRNA) or a part thereof. In some embodiments, the use of different sgRNAs allows the compositions and methods of the disclosure to be used with (e.g., targeted to) different target genes (e.g., target endogenous genes) or target gene regulatory sequences.

Prokaryotic CRISPR-Cas (Clustered regularly interspaced short palindromic repeats-CRISPR associated) systems, for example, Class II CRISPR-Cas systems such as Cas9 and Cpf1, can be repurposed as a tool for regulation of gene expression, epigenome editing, and chromatin looping in compositions and methods of the disclosure. Nuclease-deactivated Cas (dCas) proteins complexed with heterologous gene effectors can allow for regulation of expression of target genes (e.g., target endogenous genes) adjacent to a site bound by the dCas.

In some embodiments, the actuator moiety can comprise a CRISPR-associated (Cas) protein or a Cas nuclease that functions in a non-naturally occurring CRISPR (Clustered Regularly Interspaced Short Palindromic Repeats)/Cas (CRISPR-associated) system. In bacteria, this system can provide adaptive immunity against foreign DNA.

In a wide variety of organisms including diverse mammals, animals, plants, microbes, and yeast, a CRISPR/Cas system (e.g., modified and/or unmodified) can be utilized as a genome engineering tool, or can be modified to direct specific binding of engineered proteins to target loci as disclosed herein. A CRISPR/Cas system can comprise a guide nucleic acid such as a guide RNA (gRNA) complexed with a Cas protein for targeted regulation of gene expression and/or activity or nucleic acid binding. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA.

In some embodiments, the actuary moiety, such as CRISPR/Cas system comprising a guide RNA (gRNA) complexed with a Cas protein, can be configured to bind to a non-coding region or domain of the endogenous target gene. In some embodiments, a non-coding region or domain can comprise a regulatory region (e.g., regulatory sequence) that controls gene expression. In some embodiments, a non-coding region or domain can comprise promoter regions and enhancer regions. In some embodiments, the actuator moiety can be configured to bind to a domain that is free of one or more nucleotide mutations that cause the ocular disease. In some embodiments, the actuator moiety can be configured to bind to a non-coding region or domain of the endogenous target gene, wherein the domain is free of one or more nucleotide mutations that causes the ocular disease. The nucleotide mutations that cause ocular disease can comprise point mutations, insertions, deletions, substitution, or any combination thereof. Non-limiting example of ocular diseases caused by nucleotide mutations comprises retinitis pigmentosa, which is caused by amino acid substitutions at positions 68 (Cytosine (C) to Adenosine (A)), 137 (Thymine (T) to Guanine (G)), 170 (C to G), 316 (G to T), 318 (A to G), 329 (G to T), 403 (C to T), 491 (C to T), 509 (C to G), 511 (C to T), 512 (C to A), 541 (G to A), 629 (C to G), and/or 647 (C to T). In some embodiments, retinitis pigmentosa can be caused by as deletions at positions 995-1011 and/or the addition of AAA at position 1068 (based on cDNA sequence numbering with the first nucleotide of the first codon as 1). By avoiding the domain that contains one or more nucleotide mutations, the actuator moiety can target alleles of both mutated and non-mutated endogenous target gene, thus overcoming heterozygosity and increasing therapeutic efficacy. Additionally, the actuator moiety can increase specificity by avoiding off-target effects and reducing the risk of unwanted side effects.

Table 4 provides an exemplary list of gRNA spacer sequence that can bind to an endogenous target gene described herein (e.g., Rhodopsin). A spacer sequence of gRNA as described herein can comprise a polynucleotide sequence (e.g., a consecutive-polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to a polynucleotide sequence selected from Table 4 or a complementary sequence thereof (e.g., one or more members selected from the group consisting of SEQ ID NOs: 700-729).

Table 5 provides an additional exemplary list of gRNA spacer sequence that can bind to an endogenous target gene described herein (e.g., Rhodopsin). A spacer sequence of gRNA as described herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to a polynucleotide sequence selected from Table 5 or a complementary sequence thereof (e.g., one or more members selected from the group consisting of SEQ ID NOs: 730-976). In some cases, the spacer sequence of the guide nucleic acid can target a positive-sense strand (+) of the endogenous target gene. In some cases, the spacer sequence of the guide nucleic acid can target a negative-sense strand (−) of the endogenous target gene.

In an aspect, the present disclosure provides a system comprising a heterologous nucleic acid molecule exhibiting specific binding to a target polynucleotide sequence of a chromosomal gene encoding Rhodopsin, to decrease expression level of the Rhodopsin in a cell, wherein the target polynucleotide sequence (i) is part of a non-coding region of the chromosomal gene, and (ii) exhibits at least about 70% sequence identity to the polynucleotide sequence of any one of SEQ ID NOs. 700-976. In an aspect, the present disclosure provides a method comprising contacting a cell with a vector comprising a heterologous nucleic acid molecule exhibiting specific binding to a target polynucleotide sequence of a chromosomal gene encoding Rhodopsin, wherein the target polynucleotide sequence (i) is part of a non-coding region of the chromosomal gene, and (ii) exhibits at least about 70% sequence identity to the polynucleotide sequence of any one of SEQ ID NOs. 700-976.

The systems (e.g., the heterologous polypeptide and/or a guide nucleic acid) and methods thereof as provided herein can target (e.g., bind) at least one target polynucleotide sequence (e.g., a consecutive polynucleotide sequence) found in the polynucleotide sequence of one or more members in Table 6. The at least one target polynucleotide sequence can comprise at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, or at least or up to about 20 target polynucleotide sequence(s). The at least one target polynucleotide sequence can have a length of at least or up to about 6 nucleobases, at least or up to about 8 nucleobases, at least or up to about 10 nucleobases, at least or up to about 12 nucleobases, at least or up to about 16 nucleobases, at least or up to about 18 nucleobases, at least or up to about 20 nucleobases, at least or up to about 22 nucleobases, at least or up to about 24 nucleobases, at least or up to about 26 nucleobases, at least or up to about 28 nucleobases, at least or up to about 30 nucleobases, at least or up to about 32 nucleobases, at least or up to about 34 nucleobases, at least or up to about 36 nucleobases, at least or up to about 38 nucleobases, at least or up to about 40 nucleobases, at least or up to about 45 nucleobases, or at least or up to about 50 nucleobases.

In some cases, at least a portion of a positive-sense strand (+) of the endogenous target gene can be targeted. The at least the portion of the positive-sense strand can comprise a polynucleotide sequence that exhibits at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or substantially about 100% sequence identity to a consecutive polynucleotide sequence found in any one of SEQ ID NOs: 977 to 980.

In some cases, at least a portion of a negative-sense strand (−) of the endogenous target gene can be targeted. The at least the portion of the negative-sense strand can comprise a polynucleotide sequence that exhibits at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or substantially about 100% sequence identity to a consecutive polynucleotide sequence found in any one of SEQ ID NOs: 981 to 984.

In some cases, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity.

In some embodiments, the actuator moiety can comprise a Cas protein that forms a complex with a guide nucleic acid, such as a guide RNA or a part thereof. In some embodiments, the actuator moiety can comprise a Cas protein that forms a complex with a single guide nucleic acid, such as a single guide RNA (sgRNA). In some embodiments, the actuator moiety can comprise a RNA-binding protein (RBP) optionally complexed with a guide nucleic acid, such as a guide RNA (e.g., sgRNA), which is able to form a complex with a Cas protein. In some embodiments, the actuator moiety can comprise a nuclease-null DNA binding protein derived from a DNA nuclease that can induce transcriptional activation or repression of a target DNA sequence. In some embodiments, the actuator moiety can comprise a nuclease-null RNA binding protein derived from a RNA.

In some embodiments, a guide nucleic acid used in compositions and methods of the disclosure can comprise a spacer sequence that can bind to an endogenous target gene described herein. The spacer sequence can be, for example, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 nucleotides.

In some embodiments, a spacer sequence of a guide nucleic acid used in compositions and methods of the disclosure is at most at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, or at most 40 nucleotides.

In some embodiments, a spacer sequence of a guide nucleic acid used in compositions and methods of the disclosure is between about 8 and about 40 nucleotides, between about 10 and about 40 nucleotides, between about 11 and about 40 nucleotides, between about 12 and about 40 nucleotides, between about 13 and about 40 nucleotides, between about 14 and about 40 nucleotides, between about 15 and about 40 nucleotides, between about 16 and about 40 nucleotides, between about 17 and about 40 nucleotides, between about 18 and about 40 nucleotides, between about 19 and about 40 nucleotides, between about 20 and about 40 nucleotides, between about 22 and about 40 nucleotides, between about 24 and about 40 nucleotides, between about 26 and about 40 nucleotides, between about 28 and about 40 nucleotides, between about 30 and about 40 nucleotides, between about 8 and about 30 nucleotides, between about 10 and about 30 nucleotides, between about 11 and about 30 nucleotides, between about 12 and about 30 nucleotides, between about 13 and about 30 nucleotides, between about 14 and about 30 nucleotides, between about 15 and about 30 nucleotides, between about 16 and about 30 nucleotides, between about 17 and about 30 nucleotides, between about 18 and about 30 nucleotides, between about 19 and about 30 nucleotides, between about 20 and about 30 nucleotides, between about 22 and about 30 nucleotides, between about 24 and about 30 nucleotides, between about 26 and about 30 nucleotides, between about 28 and about 30 nucleotides, between about 8 and about 25 nucleotides, between about 10 and about 25 nucleotides, between about 11 and about 25 nucleotides, between about 12 and about 25 nucleotides, between about 13 and about 25 nucleotides, between about 14 and about 25 nucleotides, between about 15 and about 25 nucleotides, between about 16 and about 25 nucleotides, between about 17 and about 25 nucleotides, between about 18 and about 25 nucleotides, between about 19 and about 25 nucleotides, between about 20 and about 25 nucleotides, between about 22 and about 25 nucleotides, between about 24 and about 25 nucleotides, between about 8 and about 20 nucleotides, between about 10 and about 20 nucleotides, between about 11 and about 20 nucleotides, between about 12 and about 20 nucleotides, between about 13 and about 20 nucleotides, between about 14 and about 20 nucleotides, between about 15 and about 20 nucleotides, between about 16 and about 20 nucleotides, between about 17 and about 20 nucleotides, between about 18 and about 20 nucleotides, between about 19 and about 20 nucleotides, between about 8 and about 18 nucleotides, between about 10 and about 18 nucleotides, between about 11 and about 18 nucleotides, between about 12 and about 18 nucleotides, between about 13 and about 18 nucleotides, between about 14 and about 18 nucleotides, between about 15 and about 18 nucleotides, between about 16 and about 18 nucleotides, between about 8 and about 16 nucleotides, between about 10 and about 16 nucleotides, between about 11 and about 16 nucleotides, between about 12 and about 16 nucleotides, between about 13 and about 16 nucleotides, between about 14 and about 16 nucleotides, or between about 15 and about 16 nucleotides. In some embodiments, a guide nucleic acid can be a guide RNA or a part thereof.

Non-limiting examples of a guide RNA scaffold sequence are provided in Table 2. In some embodiments, the guide RNA scaffold sequence can comprise a polynucleotide sequence (e.g., a consecutive polynucleotide sequence) that exhibits at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or substantially about 100% sequence identity to the polynucleotide sequence of one or more members selected from Table 2 (e.g., one or more members selected from the group consisting of SEQ ID NOs. 500-596).

In some cases, a guide RNA may comprise, from 5′ to 3′, (i) the polynucleotide sequence of one or more members selected from Table 2 and (ii) a spacer sequence. In some cases, a guide RNA may comprise, from 5′ to 3′, (i) the polynucleotide sequence of SEQ ID NO: 530, (ii) a spacer sequence, and (ii) the polynucleotide sequence of TT. In some cases, a guide RNA may comprise, from 5′ to 3′, (i) the polynucleotide sequence of SEQ ID NO: 532, (ii) a spacer sequence, and (ii) the polynucleotide sequence of TTTTA. In some cases, a guide RNA may comprise, from 5′ to 3′, (i) the polynucleotide sequence of SEQ ID NO: 534, (ii) a spacer sequence, and (ii) the polynucleotide sequence of TTTTG. In some cases, a guide RNA may comprise, from 5′ to 3′, (i) the polynucleotide sequence of SEQ ID NO: 536, (ii) a spacer sequence, and (ii) the polynucleotide sequence of SEQ ID NO: 537. In some cases, a guide RNA may comprise, from 5′ to 3′, (i) the polynucleotide sequence of SEQ ID NO: 538, (ii) a spacer sequence, and (ii) the polynucleotide sequence of SEQ ID NO: 539. In some cases, a guide RNA may comprise, from 5′ to 3′, (i) the polynucleotide sequence of SEQ ID NO: 541, (ii) a spacer sequence, and (ii) the polynucleotide sequence of SEQ ID NO: 542. In some cases, a guide RNA may comprise, from 5′ to 3′, (i) the polynucleotide sequence of SEQ ID NO: 543, (ii) a spacer sequence, and (ii) the polynucleotide sequence of SEQ ID NO: 544. In some cases, a guide RNA may comprise, from 5′ to 3′, (i) the polynucleotide sequence of SEQ ID NO: 549, (ii) a spacer sequence, and (ii) the polynucleotide sequence of SEQ ID NO: 550. In some cases, a guide RNA may comprise, from 5′ to 3′, (i) the polynucleotide sequence of SEQ ID NO: 551, (ii) a spacer sequence, and (ii) the polynucleotide sequence of SEQ ID NO: 552. In some cases, a guide RNA may comprise, from 5′ to 3′, (i) the polynucleotide sequence of SEQ ID NO: 554, (ii) a spacer sequence, and (ii) the polynucleotide sequence of TTTTA. In some cases, a guide RNA may comprise, from 5′ to 3′, (i) the polynucleotide sequence of SEQ ID NO: 564, (ii) a spacer sequence, and (ii) the polynucleotide sequence of SEQ ID NO: 550. In some cases, a guide RNA may comprise, from 5′ to 3′, (i) the polynucleotide sequence of SEQ ID NO: 565, (ii) a spacer sequence, and (ii) the polynucleotide sequence of SEQ ID NO: 550.

Non-limiting examples of a guide RNA scaffold fragment sequence are provided in Table 3. In some embodiments, the guide RNA scaffold sequence can comprise a polynucleotide sequence (e.g., a consecutive polynucleotide sequence) that exhibits at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or substantially about 100% sequence identity to the polynucleotide sequence of one or more members selected from Table 3 (e.g., one or more members selected from the group consisting of SEQ ID NOs. 597-601). Any suitable CRISPR/Cas system can be used. A CRISPR/Cas system can be referred to using a variety of naming systems. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

When a actuator moiety can comprise a Cas protein or derivative thereof, the Cas protein or derivative thereof can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein or fragment thereof that is fused to other proteins or polypeptides. A Cas protein can be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Casoe, Casof, Cas7, Cas8a, Cas8a1, Cas8a2, Cas8b, Cas8c, Cas9 (Csnl or Csx12), Cas10, Cas10d, Cas10, Cas10d, CasF, CasG, CasH, Cpf1, Csyl, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, Cul966, Cas13a, Cas13b, Cas13c, Cas13d, Cas13X, Cas13Y, Cas14 (e.g., Cas14 variants, such as Cas14a, Cas14b, Cas14c, etc.) and homologs or modified versions thereof.

A Cas protein or fragment or derivative thereof can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas nap hthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinellasuccinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wildtype or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein (e.g., a wild-type version of Cas14). A Cas protein can be a polypeptide with at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a wild type Cas protein. A Cas protein can be a polypeptide with at most about 5%, at most about 10%, at most about 20%, at most about 30%, at most about 40%, at most about 50%, at most about 60%, at most about 70%, at most about 80%, at most about 90%, or at most about 100% sequence identity and/or sequence similarity to a wild type exemplary Cas protein. Variants or fragments can comprise at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as DNase domains. For example, a Cas9 protein can comprise a RuvC-like nuclease domain and/or an HNH-like 20 nuclease domain. The in a nuclease active form of Cas9, RuvC and HNH domains can each cut a different strand of double-stranded DNA to make a double-stranded break in the DNA. A Cas protein can comprise only one nuclease domain (e.g., Cpf1 comprises RuvC domain but lacks HNH domain). In some embodiments, nuclease domains are absent. In some embodiments, nuclease domains are present but inactive or have reduced or minimal activity. In some embodiments, nuclease domains are present and active.

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. If all of the nuclease domains of a Cas protein (e.g., both RuvC and HNH nuclease domains in a Cas9 protein; RuvC nuclease domain in a Cpf1 protein) are deleted or mutated, the resulting Cas protein can have a reduced or no ability to cleave both strands of a double-stranded DNA. An example of a mutation that can convert a Cas9 protein into a nickase is a D10A (aspartate to alanine at position 10 of Cas9) mutation in the RuvC domain of Cas9 from S. pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A nuclease dead Cas protein can comprise one or more mutations relative to a wild-type version of the protein. The mutation can result in no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or more of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild type S. pyogenes Cas9 polypeptide, for example, as determined by sequence and/or structural alignment.

A Cas protein can comprise an amino acid sequence having at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a nuclease domain (e.g., RuvC domain, HNH domain) of a wild-type Cas protein.

A Cas protein, variant or derivative thereof can be modified to enhance regulation of gene expression by compositions and methods of the disclosure, e.g., as part of a complex disclosed herein. A Cas protein can be modified to increase or decrease nucleic acid binding affinity, nucleic acid binding specificity, enzymatic activity, and/or binding to other factors, such as heterodimerization or oligomerization domains and induce ligands. Cas proteins can also be modified to change any other activity or property of the protein, such as stability. For example, one or more nuclease domains of the Cas protein can be modified, deleted, or inactivated, or a Cas protein can be truncated to remove domains that are not essential for the desired function of the protein or complex. A Cas protein can be modified to modulate (e.g., enhance or reduce) the activity of the Cas protein for regulating gene expression by a complex of the disclosure that comprises a heterologous gene effector.

For example, a Cas protein can be coupled (e.g., fused, covalently coupled, or non-covalently coupled) to a heterologous gene effector (e.g., an epigenetic modification domain, a transcriptional activation domain, and/or a transcriptional repressor domain). A Cas protein can be coupled (e.g., fused, covalently coupled, or non-covalently coupled) to an oligomerization or dimerization domain as disclosed herein (e.g., a heterodimerization domain). A Cas protein can be coupled (e.g., fused, covalently coupled, or non-covalently coupled) to a heterologous polypeptide that provides increased or decreased stability. A Cas protein can be coupled (e.g., fused, covalently coupled, or non-covalently coupled) to a sequence that can facilitate degradation of the Cas protein or a complex containing the Cas protein, for example, a degron, such as an inducible degron (e.g., auxin inducible).

A Cas protein can be coupled (e.g., fused, covalently coupled, or non-covalently coupled) to any suitable number of partners, for example, at least one, at least two, at least three, at least four, or at least five, at least six, at least seven, or at least 8 partners. In some embodiments, a Cas protein of the disclosure is coupled (e.g., fused, covalently coupled, or non-covalently coupled) to at most two, at most three, at most four, at most five, at most six, at most seven, at most eight, or at most ten partners. In some embodiments, a Cas protein of the disclosure is coupled (e.g., fused, covalently coupled, or non-covalently coupled) to 1-5, 1-4, 1-3, 1-2, 2-5, 2-4, 2-3, 3-5, 3-4, or 4-5 partners. In some embodiments, a Cas protein of the disclosure is coupled (e.g., fused, covalently coupled, or non-covalently coupled) to one partner. In some embodiments, a Cas protein of the disclosure is coupled (e.g., fused, covalently coupled, or non-covalently coupled) to two partners. In some embodiments, a Cas protein of the disclosure is coupled (e.g., fused, covalently coupled, or non-covalently coupled) to three partners. In some embodiments, a Cas protein of the disclosure is coupled (e.g., fused, covalently coupled, or non-covalently coupled) to four partners. In some embodiments, a Cas protein of the disclosure is coupled (e.g., fused, covalently coupled, or non-covalently coupled) to five partners. In some embodiments, a Cas protein of the disclosure is coupled (e.g., fused, covalently coupled, or non-covalently coupled) to six partners.

A Cas protein can be a fusion protein, e.g., a fusion comprising the Cas protein and one or more of the partners as disclosed herein. The fused domain or heterologous polypeptide can be located at the N-terminus, the C-terminus, or internally within the Cas protein.

A partner of the Cas protein (e.g., covalently or non-covalently coupled to a dCas protein as disclosed herein) can be a transcriptional effector (e.g., a transcriptional activator or a transcriptional repressor). The transcriptional effector can be heterologous to the cell as provided herein.

In some embodiments, the Cas protein and the transcriptional effector (e.g., transcriptional activator) can be fused in a single polypeptide sequence. The Cas protein and the transcriptional effector can be fused directly to one another. Alternatively, the Cas protein and the transcriptional effector can be fused via a peptide linker (or an amino acid linker) that is heterologous to the Cas protein and the transcriptional activator. The peptide linker can be derived from a natural polypeptide sequence. Alternatively, the peptide linker can be a synthetic sequence. The peptide linker can have a length of at least or up to about 1 amino acid residue, at least or up to about 2 amino acid residues, at least or up to about 3 amino acid residues, at least or up to about 4 amino acid residues, at least or up to about 5 amino acid residues, at least or up to about 10 amino acid residues, at least or up to about 15 amino acid residues, at least or up to about 20 amino acid residues, at least or up to about 25 amino acid residues, at least or up to about 30 amino acid residues, at least or up to about 35 amino acid residues, at least or up to about 40 amino acid residues, at least or up to about 45 amino acid residues, at least or up to about 50 amino acid residues, at least or up to about 60 amino acid residues, at least or up to about 70 amino acid residues, at least or up to about 80 amino acid residues, at least or up to about 90 amino acid residues, or at least or up to about 100 amino acid residues. In some cases, the peptide linker can be a GS linker.

The term “GS linker” or “GS linker sequence,” as used interchangeably herein, generally refers to a peptide linker that mainly comprises glycine and serine residues. Particularly, at least or up to about 60%, at least or up to about 65%, at least or up to about 70%, at least or up to about 75%, at least or up to about 80%, at least or up to about 85%, at least or up to about 90%, at least or up to about 95% or substantially about 100% of the amino acid residues in the GS linker sequence can be selected from glycine and serine residues. The GS linker sequence according to the present invention can, for example, comprise from about 1 to about 50 amino acid residues, from about 1 to about 45 amino acid residues, from about 1 to about 40 amino acid residues, from about 1 to about 35 amino acid residues, or from about 1 to about 30 amino acid residues, in total. In some cases, the GS linker sequence may not comprise about 10, about 5, about 4, about 3, about 2 or about 1 amino acid residue(s) other than glycine or serine.

In some embodiments, the transcriptional effector can be a histone epigenetic modifier (or a histone modifier). In some cases, the histone epigenetic modifier can modulate histones through methylation (e.g., a histone methylation modifier, such as an amino acid methyltransferase, e.g., KRAB). In some cases, the histone epigenetic modifier can modulate histones through acetylation. In some cases, the histone epigenetic modifier can modulate histones through phosphorylation. In some cases, the histone epigenetic modifier can modulate histones through ADP-ribosylation. In some cases, the histone epigenetic modifier can modulate histones through glycosylation. In some cases, the histone epigenetic modifier can modulate histones through SUMOylation. In some cases, the histone epigenetic modifier can modulate histones through ubiquitination. In some cases, the histone epigenetic modifier can modulate histones by remodeling histone structure, e.g., via an ATP hydrolysis-dependent process.

In some embodiments, the transcriptional effector can be a gene epigenetic modifier (or a gene modifier). In some cases, a gene modifier can modulate genes through methylation (e.g., a gene methylation modifier, such as a DNA methyltransferase or DNMT). In some cases, a gene modifier can modulate genes through acetylation.

In some embodiments, the transcriptional effector is from a family of related histone acetyltransferases. Non-limiting examples of histone acetyltransferases include GNAT subfamily, MYST subfamily, p300/CBP subfamily, HAT1 subfamily, GCN5, PCAF, Tip60, MOZ, MORF, MOF, HBO1, p300, CBP, HAT1, ATF-2, SRC1, and TAFII250.

In some embodiments, the transcriptional effector is from a histone epigenetic modifier (e.g., a histone lysine methyltransferase, a histone lysine demethylase, or a DNA methylase). Non-limiting examples of histone epigenetic modifier include EZH subfamily, Non-SET subfamily, Other SET subfamily, PRDM subfamily, SET1 subfamily, SET2 subfamily, SUV39 subfamily, SYMD subfamily, ASHIL, EHMT1, EHMT2, EZH1, EZH2, MLL, MLL2, MLL3, MLL4, MLL5, NSD1, NSD2, NSD3, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, SET1, SET1L, SET2L, SETD2, SETD3, SETD4, SETD5, SETD6, SETD7, SETD8, SETDB1, SETDB2, SETMAR, SUV39H1, SUV39H2, SUV420H1, SUV420H2, SYMD1, SYMD2, SYMD3, SYMD4, and SYMD5.

Examples of proteins (or fragments thereof) that can be used as a fusion partner to increase transcription include but are not limited to: transcriptional activators such as VP16, VP64, VP48, VP160, p65 subdomain (e.g., from NFKB), and activation domain of EDLL and/or TAL activation domain (e.g., for activity in plants); and histone epigenetic modifier such as SETIA, SET1B, MLL1 to 5, ASH1, SYMD2, NSD1, JHDM2a/b, UTX, JMJD3, GCN5, PCAF, CBP, p300, TAF1, TIP60/PLIP, MOZMYST3, MORFMYST4, SRC1, ACTR, PI 60, CLOCK, Ten-Eleven Translocation (TET) dioxygenase 1 (TET1CD), TET1, DME, DML1, DML2, ROS1, and the like.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARJD 1 A/RBP2, JARIDIB/PLU-1, JARID 1C/SMCX, JARIDID/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

A Cas protein can be provided in any form. For example, a Cas protein can be provided in the form of a protein, such as a Cas protein alone or complexed with a guide nucleic acid as a ribonucleoprotein. A Cas protein can be provided in a complex, for example, complexed with a guide nucleic acid and/or one or more heterologous gene effectors of the disclosure. A Cas protein can be provided in the form of a nucleic acid encoding the Cas protein, such as an RNA (e.g., messenger RNA (mRNA)), or DNA. The nucleic acid encoding the Cas protein can be codon optimized for efficient translation into protein in a particular cell or organism.

Nucleic acids encoding Cas proteins, fragments, or derivatives thereof can be stably integrated in the genome of a cell. Nucleic acids encoding Cas proteins can be operably linked to a promoter, for example, a promoter that is constitutively or inducibly active in the cell. Nucleic acids encoding Cas proteins can be operably linked to a promoter in an expression construct. Expression constructs can include any nucleic acid constructs capable of directing expression of a gene or other nucleic acid sequence of interest (e.g., a Cas gene) and which can transfer such a nucleic acid sequence of interest to a target cell.

In some embodiments, a Cas protein, variant or derivative thereof is a nuclease dead Cas (dCas) protein. A dead Cas protein can be a protein that lacks nucleic acid cleavage activity.

A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive, “deactivated” and/or “dead” (abbreviated by “d”). A dead Cas protein (e.g., dCas, dCas9, dCas14) can bind to a target polynucleotide but may not cleave or minimally cleaves the target polynucleotide. In some aspects, a dead Cas protein is a dead Cas14 protein. In some aspects, a dead Cas protein is a not a dead Cas14 protein.

A dCas polypeptide (e.g., dCas14 polypeptide) can associate with a single guide RNA (sgRNA) to activate or repress transcription of a target gene (e.g., target endogenous gene), for example, in combination with heterologous gene effector(s) disclosed herein. sgRNAs can be introduced into cells expressing the Cas or variant thereof, as provided herein. In some cases, such cells can contain one or more different sgRNAs that target the same target gene (e.g., target endogenous gene) or target gene regulatory sequence. In other cases, the sgRNAs target different nucleic acids in the cell (e.g., different target genes, different target gene regulatory sequences, or different sequences within the same target gene or target gene regulatory sequence).

Enzymatically inactive can refer to a nuclease that can bind to a nucleic acid sequence in a polynucleotide in a sequence-specific manner, but will not cleave a target polynucleotide or will cleave it at a substantially reduced frequency. An enzymatically inactive guide moiety can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a comparable wild-type activity (e.g., nucleic acid cleaving activity, wild-type Cas9 or wild-type Cas14 activity).

In some embodiments, the actuator moiety as disclosed herein does not contain a nucleic acid-guided targeting system. For example, the actuator moiety can include proteins that bind to a target gene (e.g., target endogenous gene) or target gene regulatory sequence based on protein structural features, such as certain nucleases disclosed herein.

In some embodiments, the wild-type Cas protein that the engineered Cas protein is a modification of has a native amino acid sequence with a length of less than 800 amino acids (e.g., Cas14 or a variant thereof). This relatively small size provides several advantages to the provided engineered Cas protein. For example, the small size can allow the Cas protein to be delivered to a host cell, e.g., a cell of a human patient, via a single adeno-associated virus delivery system that would be otherwise incapable of delivering a larger protein. The native amino acid sequence can have a length that is, for example, between 500 amino acids and 700 amino acids, e.g., between 500 amino acids and 620 amino acids, between 540 amino acids and 660 amino acids, between 560 amino acids and 680 amino acids, or between 580 amino acids and 700 amino acids. In terms of upper limits, the native amino acid sequence can have a length that is less than 700 amino acids, e.g., less than 680 amino acids, less than 660 amino acids, less than 640 amino acids, less than 620 amino acids, less than 600 amino acids, less than 580 amino acids, less than 560 amino acids, less than 540 amino acids, or less than 520 amino acids. In terms of lower limits, the native amino acid sequence can have an length that is greater than 500 amino acids, e.g., greater than 520 amino acids, greater than 540 amino acid, greater than 560 amino acids, greater than 580 amino acids, greater than 600 amino acids, greater than 620 amino acids, greater than 640 amino acids, greater than 660 amino acids, or greater than 700 amino acids. Larger lengths, e.g., greater than 700 amino acids, and smaller lengths, e.g., less than 500 amino acids, are also contemplated.

In some embodiments, the modified amino acid sequence of the engineered Cas protein includes one or more substitutions in the native amino acid sequence, where the positions of at least some of these substitutions follow one or more particular rules determined to have surprising advantages for the characteristics of the engineered Cas protein. For example, the particular substitution rules have been selected for their ability to produce engineered Cas proteins capable of functioning within eukaryotic cells. According to these particular rules, all or some of the one or more substitutions in the native amino acid sequence are either (1) within or no more than 30 amino acids downstream of a (D/E/K/N)X(R/F) (E/K) N motif of the native amino acid sequence, (2) at or no more than 30 amino acids upstream or downstream of position 241 of the native amino acid sequence, (3) at or no more than 30 amino acids upstream or downstream of position 516 of the native amino acid sequence, and/or (4) having an electrically charged amino acid in the native amino acid sequence.

In some embodiments, the native amino acid sequence includes a (D/E/K/N)X(R/F) (E/K) N motif, and the modified amino acid sequence includes one or more substitutions at positions within or no more than 30 amino acids upstream or downstream of the motif. The modified amino acid sequence can include, for example, one, two, three, four, five, six, seven, eight, nine, ten, or more than ten substitutions within or no more than 30 amino acids upstream or downstream of the motif. At least one of the one or more substitutions to the native amino acid sequence can be, for example, within or no more than 28 amino acids, 26 amino acids, 24 amino acids, 22 amino acids, 20 amino acids, 18 amino acids, 16 amino acids, 14 amino acids, 12 amino acids, or 10 amino acids of the motif. In some embodiments, at least one of the one or more substitutions within or no more than 30 amino acids upstream or downstream of the motif is to an R, A, S, or G. In some embodiments, each of the one or more substitutions within or no more than 30 amino acids upstream or downstream of the motif is independently to an R, A, S, or G. In some embodiments, all of the substitutions to the native amino acid sequence are at positions within or no more than 30 amino acids upstream or downstream of the motif.

Some embodiments of the present disclosure are directed to a Cas protein that is not a variant of CasX.

Some embodiments of the present disclosure are directed to small Cas-based regulation of gene expression, such as at the transcriptional and/or translational level. Small Cas proteins can be targeted to DNA and/or RNA, and are much smaller than typical CRISPR effectors, e.g., ranging in size from about 400 amino acids to about 700 amino acids. The small size of can allow such Cas proteins and/or effector domain fusions thereof to be paired with a CRISPR array encoding multiple guide RNAs while remaining under the packaging size limit of various delivery vehicles, such as the versatile adeno-associated virus (AAV) delivery vehicle or non-viral delivery vehicles (e.g., lipid nanoparticles), for primary cell and in vivo delivery.

In some embodiments, the Cas protein or a variant thereof as provided herein (e.g., a variant of SEQ ID NO: 1 as disclosed herein) can have a size of at most about 800 amino acids, at most about 780 amino acids, at most about 760 amino acids, at most about 750 amino acids, at most about 740 amino acids, at most about 720 amino acids, at most about 700 amino acids, at most about 680 amino acids, at most about 660 amino acids, at most about 650 amino acids, at most about 640 amino acids, at most about 620 amino acids, at most about 600 amino acids, at most about 580 amino acids, at most about 560 amino acids, at most about 550 amino acids, at most about 540 amino acids, at most about 520 amino acids, at most about 500 amino acids, 480 amino acids, at most about 460 amino acids, at most about 450 amino acids, at most about 440 amino acids, at most about 420 amino acids, at most about 400 amino acids, or less.

Non-limiting examples of Cas protein are provided in Table 1. In some embodiments, the Cas protein or the deactivated Cas protein (dCas) as provided herein can comprise a polypeptide sequence (e.g., a consecutive polypeptide sequence) that exhibits at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or substantially about 100% sequence identity to the polypeptide sequence of one or more members selected from Table 1 (e.g., one or more members selected from the group consisting of SEQ ID NOs. 1-201).

In some embodiments, the Cas protein or a variant thereof, as provided herein, can comprise the amino acid sequence having at least about 60%, at least about 65%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to the amino acid sequence of SEQ ID NO: 1. Cas protein or a variant thereof, as provided herein, can comprise the amino acid sequence having at most about 100%, at most about 99%, at most about 98%, at most about 97%, at most about 96%, at most about 95%, at most about 94%, at most about 93%, at most about 92%, at most about 91%, at most about 90%, at most about 89%, at most about 88%, at most about 87%, at most about 86%, at most about 85%, at most about 84%, at most about 83%, at most about 82%, at most about 81%, at most about 80%, at most about 79%, at most about 78%, at most about 77%, at most about 76%, at most about 75%, at most about 74%, at most about 73%, at most about 72%, at most about 71%, at most about 70%, at most about 65%, at most about 60%, or less sequence identity to the amino acid sequence of SEQ ID NO: 1.

In some embodiments, a Cas protein or a variant thereof as disclosed herein can exhibit a greater cationic charge (e.g., at least 1, at least 2, at least 3, at least 4, at least 5, at least 6, at least 7, at least 8, at least 9, at least 10, or more cationic charges) as compared to the wild-type Cas14. The enhanced cationic charge can (i) enhance complexation of the Cas protein to the guide nucleic acid and/or (ii) enhance complexation of the Cas protein to the target polynucleotide sequence (e.g., endogenous target polynucleotide sequence). In some cases, the Cas protein can comprise one or more substitutions for the enhanced cationic charge. The one or more substitutions at positions within or no more than 30 amino acids upstream or downstream of the (D/E/K/N)X(R/F) (E/K) N motif of the native amino acid sequence can include, for example, one or more substitutions at positions selected from positions 143, 147, 151, and 154 of the native amino acid sequence. In some embodiments, e.g., when the native amino acid sequence is the sequence of SEQ ID NO: 1, the one or more substitutions include substitutions are at one or more positions selected from D143, T147, E151, and K154. In some embodiments, e.g., when the native amino acid sequence is the sequence of SEQ ID NO: 1, the one or more substitutions include one or more substitutions selected from D143R, T147R, E151R, and K154R.

In some embodiments, the modified amino acid sequence includes one or more substitutions at or no more than 30 amino acids upstream or downstream of position 241 of the native amino acid sequence. The modified amino acid sequence can include, for example, one, two, three, four, five, six, seven, eight, nine, ten, or more than ten substitutions within or no more than 30 amino acids upstream or downstream of position 241. At least one of the one or more substitutions to the native amino acid sequence can be, for example, within or no more than 28 amino acids, 26 amino acids, 24 amino acids, 22 amino acids, 20 amino acids, 18 amino acids, 16 amino acids, 14 amino acids, 12 amino acids, or 10 amino acids of position 241. In some embodiments, at least one of the one or more substitutions within or no more than 30 amino acids upstream or downstream of position 241 is to an R, A, S, or G. In some embodiments, each of the one or more substitutions within or no more than 30 amino acids upstream or downstream of position 241 is independently to an R, A, S, or G. In some embodiments, all of the substitutions to the native amino acid sequence are at positions within or no more than 30 amino acids upstream or downstream of position 241.

In some embodiments, e.g., when the native amino acid sequence is the sequence of SEQ ID NO: 1, the one or more substitutions at positions having an electrically charged amino include substitutions are at one or more positions selected from K11, K73, D143, E151, K154, E241, D318, K330, K457, E425, E462, E507, E527, and E528. In some embodiments, e.g., when the native amino acid sequence is the sequence of SEQ ID NO: 1, the one or more substitutions include one or more substitutions selected from K11R, K73R, D143R, E151R, K154R, E241R, D318R, K330R, E425N, K457R, E462R, E507R, E527R, and E528R. In some embodiments, the modified amino acid sequence includes a D143R substitution. In some embodiments, the only substitution in the modified amino acid sequence is D143R.

In some embodiments, the modified amino acid sequence of the engineered Cas protein includes two substitutions in the native amino acid sequence. In some embodiments, the modified amino acid sequence has exactly two substitutions in the native amino acid sequence. In some embodiments, the modified amino acid sequence includes two substitutions at positions selected from positions 143, 147, 151, 154, 241, 330, 425, 504, 507, 516, 519, 527, and 528. In some embodiments, the modified amino acid sequence has exactly two substitutions, where the exactly two substitutions are at positions selected from positions 143, 147, 151, 154, 241, 330, 425, 504, 507, 516, 519, 527, and 528. In some embodiments, e.g., when the native amino acid sequence is the sequence of SEQ ID NO: 1, the modified amino acid sequence includes two substitutions at positions selected from D143, T147, E151, K154, E241, K330, E425, N504, E507, N516, N519, E527, and E528. In some embodiments, e.g., when the native amino acid sequence is the sequence of SEQ ID NO: 1, the modified amino acid sequence has exactly two substitutions, where the exactly two substitutions are at positions selected from D143, T147, E151, K154, E241, K330, E425, N504, E507, N516, N519, E527, and E528.

In some embodiments, the modified amino acid sequence includes a substitution at position 143 and a substitution at a position selected from positions 147, 151, 154, 241, 330, 425, 504, 507, 516, 519, 527, and 528. In some embodiments, the modified amino acid includes a substitution at position 143 and exactly one other substitution, where the exactly one other substitution is at a position selected from positions 147, 151, 154, 241, 330, 425, 504, 507, 516, 519, 527, and 528. In some embodiments, e.g., when the native amino acid sequence is the sequence of SEQ ID NO: 1, the modified amino acid sequence includes a substitution at position D143 and a substitution at a position selected from positions T147, E151, K154, E241, K330R, E425N, N504, E507, N516, N519, E527, and E528. In some embodiments, e.g., when the native amino acid sequence is the sequence of SEQ ID NO: 1, the modified amino acid includes a substitution at position D143 and exactly one other substitution, where the exactly one other substitution is at a position selected from positions T147, E151, K154, E241, K330R, E425N, N504, E507, N516, N519, E527, and E528.

In some embodiments, e.g., when the native amino acid sequence is the sequence of SEQ ID NO: 1, the modified amino acid includes two substitutions selected from D143R, T147R, E151R, E151A, K154R, E241R, N504R, E507R, N516R, N519R, E527R, and E528R. In some embodiments, e.g., when the native amino acid sequence is the sequence of SEQ ID NO: 1, the modified amino acid includes exactly two substitutions, where the two substitutions are selected from D143R, T147R, E151R, E151A, K154R, E241R, N504R, E507R, N516R, N519R, E527R, and E528R. In some embodiments, e.g., when the native amino acid sequence is the sequence of SEQ ID NO: 1, the modified amino acid includes two substitutions selected from D143R/T147R, D143R/E151R, D143R/E241R, D143R/E425N, D143R/E507R, D143R/N519R, D143R/E527R, D143R/E528R, D143R/R151S, D143/R151G, and D143R/E151A. In some embodiments, e.g., when the native amino acid sequence is the sequence of SEQ ID NO: 1, the modified amino acid includes exactly two substitutions, where the two substitutions are selected from D143R/T147R, D143R/E151R, D143R/E241R, D143R/E425N, D143R/E507R, D143R/N519R, D143R/E527R, D143R/E528R, D143R/R151S, D143/R151G, and D143R/E151A. In some embodiments, the modified amino acid sequence includes a D143R substitution and a T147R substitution. In some embodiments, the only substitutions in the modified amino acid sequence are a D143R substitution and a T147R substitution.

In some embodiments, provide herein is a dCas protein or a variant thereof where one or more amino acids of the parental Cas protein from which it is derived have been altered or otherwise removed to reduce or eliminate its nuclease activity. In some embodiments, the amino acids include D326 and D510 with respect to SEQ ID NO: 1. In some embodiments, one or both of D326 and D510 are substituted with an amino acid that reduces, substantially eliminates, or eliminates nuclease activity. In some embodiments, one or both of D326 and D510 are substituted with alanine (e.g., D326A and/or D510A based on SEQ ID NO: 1). In some embodiments, the dCas protein exhibits reduced or eliminated nuclease activity, or nuclease activity is absent or substantially absent within levels of detection.

In some embodiments, the dCas protein or a variant thereof comprises the amino acid sequence of SEQ ID NO: 1 or a variant thereof having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to the amino acid sequence of SEQ ID NO: 1.

In some embodiments, according to any of the Cas protein systems described herein, the target nucleic acid is dsDNA. In such embodiments, dsDNA-targeting specificity is determined, at least in part, by two parameters: the gRNA spacer targeting a protospacer in the target dsDNA (the sequence in the target dsDNA corresponding to the gRNA spacer on the non-complementary DNA strand) and a short sequence, the protospacer-adjacent motif (PAM), located immediately 5′ (upstream) of the protospacer on the non-complementary DNA strand. In some embodiments, the PAM is 5′-TTTG-3′ or 5′-TTTA-3′. In some embodiments, the PAM is 5′-TTTG-3′. In some embodiments, the PAM is 5′-TTTA-3′.

In some embodiments, according to any of the Cas protein systems described herein, the target nucleic acid is RNA. In such embodiments, RNA-targeting specificity is determined, at least in part, by the gRNA spacer targeting a protospacer-like sequence in the target RNA (the sequence in the target RNA complementary to the gRNA spacer), and is independent of the sequence located immediately 5′ (upstream) of the protospacer-like sequence. In some embodiments, the Cas protein system is also capable of targeting a dsDNA molecule, wherein the gRNA spacer is selected such that it targets a protospacer in the target dsDNA molecule having a PAM selected from 5′-TTTG-3′ and 5′-TTTA-3′. In other embodiments, the Cas protein system is incapable of targeting a dsDNA molecule, wherein the gRNA spacer is selected such that any protospacers in the dsDNA molecule targeted by the gRNA spacer do not have a PAM selected from 5′-TTTG-3′ and 5′-TTTA-3′.

In some embodiments, a actuator moiety can comprise a zinc finger nuclease (ZFN) or a variant, fragment, or derivative thereof. ZFN can refer to a fusion between a cleavage domain, such as a cleavage domain of Fokl, and at least one zinc finger motif (e.g., at least 2, at least 3, at least 4, or at least 5 zinc finger motifs) which can bind polynucleotides such as DNA and RNA. In some embodiments, a ZFN is used in a targeting moiety of the disclosure to bind a polynucleotide (e.g., target gene or target gene regulatory sequence), but the ZFN does not cleave or substantially does not cleave the polynucleotide, e.g., a nuclease dead ZFN. A ZFN or a variant, fragment, or derivative thereof can be fused to or associated with one of more heterologous gene effectors to form a complex of the disclosure.

The heterodimerization at certain positions in a polynucleotide of two individual ZFNs in certain orientation and spacing can lead to cleavage of the polynucleotide in nuclease-active ZFN. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain.

In some embodiments, the cleavage domain of an actuator moiety comprising a ZFN comprises a modified form of a wild type cleavage domain. The modified form of the cleavage domain can comprise an amino acid change (e.g., deletion, insertion, or substitution) that reduces the nucleic acid-cleaving activity of the cleavage domain. For example, the modified form of the cleavage domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the corresponding wild-type cleavage domain. The modified form of the cleavage domain can have no substantial nucleic acid-cleaving activity. In some embodiments, the cleavage domain is enzymatically inactive.

In some embodiments, a actuator moiety can comprise a “TALEN” or “TAL-effector nuclease” or a variant, fragment, or derivative thereof. TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated Fok1 endonuclease or the catalytic domain of Fok1. In some embodiments, a TALEN is used in a targeting moiety of the disclosure to bind a polynucleotide (e.g., target gene or target gene regulatory sequence), but the TALEN does not cleave or substantially does not cleave the polynucleotide, e.g., a nuclease dead TALEN. A TALEN or a variant, fragment, or derivative thereof can be fused to or associated with one of more heterologous gene effectors to form a complex of the disclosure.

In some embodiments, a TALEN is engineered for reduced nuclease activity. In some embodiments, the nuclease domain of a TALEN comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. A TALEN or a variant, fragment, or derivative thereof can be fused to or associated with one of more heterologous gene effectors to form a complex of the disclosure.

Several mutations to Fok1 have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HDR.

A TALE or a variant, fragment, or derivative thereof can be fused to or associated with one of more heterologous gene effectors to form a complex of the disclosure. In some embodiments, the transcription activator-like effector (TALE) protein is fused to a heterologous gene effector and does not comprise a nuclease. In some embodiments, a TALEN does not cleave or substantially does not cleave the polynucleotide, e.g., a nuclease dead TALE. A TALE or a variant, fragment, or derivative thereof can be fused to or associated with one of more heterologous gene effectors to form a complex of the disclosure.

In some embodiments, the complex of the transcription activator-like effector (TALE) protein and the heterologous gene effector is designed to function as a transcriptional activator. In some embodiments, the complex of the transcription activator-like effector (TALE) protein and the heterologous gene effector is designed to function as a transcriptional repressor. For example, the DNA-binding domain of the transcription activator-like effector (TALE) protein can be fused (e.g., linked) to one or more heterologous gene effectors that comprise transcriptional activation domains, or to one or more heterologous gene effectors that comprise transcriptional repression domains.

In some embodiments, a actuator moiety can comprise a meganuclease. Meganucleases generally refer to rare-cutting endonucleases or homing endonucleases that can be highly sequence specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. A nuclease-active meganuclease can generate a double-stranded break. In some embodiments, a meganuclease is used in a targeting moiety of the disclosure to bind a polynucleotide (e.g., target gene or target gene regulatory sequence), but the meganuclease does not cleave or substantially does not cleave the polynucleotide, e.g., a nuclease dead meganuclease. A meganuclease or a variant, fragment, or derivative thereof can be fused to or associated with one of more heterologous gene effectors to form a complex of the disclosure.

The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-Msol meganuclease, I-SceI meganuclease, variants thereof, derivatives thereof, and fragments thereof.

In some embodiments, the nuclease domain of a meganuclease comprises a modified form of a wild type nuclease domain. The modified form of the nuclease domain can comprise an amino acid change (e.g., deletion, insertion, or substitution) that reduces or eliminates the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA. In some embodiments, a nuclease-inactive meganuclease is fused to or associated with one or more heterologous gene effectors to generate a complex of the disclosure.

In some embodiments, the heterologous polypeptide comprising the actuator moiety (e.g., and/or a complex comprising the heterologous polypeptide) can regulate expression and/or activity of a target gene (e.g., target endogenous gene). In some embodiments, the heterologous polypeptide and/or a complex thereof can edit the sequence of a nucleic acid (e.g., a gene and/or gene product). A nuclease-active Cas protein can edit a nucleic acid sequence by generating a double-stranded break or single-stranded break in a target polynucleotide.

In some embodiments, the heterologous polypeptide comprising the actuator moiety (e.g., and/or a complex comprising the heterologous polypeptide) can generate a double-strand break in a target polynucleotide, such as DNA. A double-strand break in DNA can result in DNA break repair which allows for the introduction of gene modification(s) (e.g., nucleic acid editing). In some embodiments, a nuclease induces site-specific single-strand DNA breaks or nicks, thus resulting in HDR.

A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided.

In some embodiments, the heterologous polypeptide comprising the actuator moiety (e.g., and/or a complex comprising the heterologous polypeptide) does not generate a double-strand break in a target polynucleotide, such as DNA. Binding of the heterologous polypeptide of the complex comprising the heterologous polypeptide (e.g., a complex comprising a dCas-effector and a guide RNA) without a nucleic acid break can be sufficient to regulate expression (e.g., enhance or suppress) of a target gene (e.g., endogenous target gene).

### Target Gene

The disclosure provides compositions, methods, and systems for modulating expression of target genes. The target genes can be one or more endogenous target genes, such as a disease causing allele, e.g., a mutant allele. For example, disclosed herein are complexes that comprise a guide moiety and one or more heterologous polypeptides comprising an actuator moiety that can increase or decrease an activity or expression level of a target gene.

In some embodiments, a target gene or regulatory sequence thereof is endogenous to a cell, for example, present in the cell's genome, or endogenous to a subject, for example, present in the subject's genome. In some embodiments, a target gene or regulatory sequence thereof is not part of an engineered reporter system.

In some embodiments, a target gene is exogenous to a host subject, for example, a pathogen target gene or an exogenous gene expressed as a result of a therapeutic intervention, such as a gene therapy and/or cell therapy. In some embodiments, a target gene is an exogenous reporter gene. In some embodiments, a target gene is an exogenous synthetic gene.

In some embodiments, the systems and methods as disclosed herein can modulate (e.g., increase or decrease) expression of a target gene (e.g., upon introducing a complex comprising the heterologous polypeptide into a cell or population of cells). In some embodiments, an expression level is an RNA expression level can be measured by, for example, RNAseq, qPCR, microarray, gene array, FISH, etc. In some embodiments, an expression level is a protein expression level can be measured by, for example, Western Blot, ELISA, multiplex immunoassay, mass spectrometry, NMR, proteomics, flow cytometry, mass cytometry, etc.

In some embodiments, the systems and methods as disclosed herein can modulate (e.g., increase or decrease) expression of a target gene (e.g., upon introducing a complex comprising the heterologous polypeptide into a cell or population of cells) by at least about 10%, at least about 20%, at least about 30%, at least about 40%, at least about 50%, at least about 60%, at least about 70%, at least about 80%, at least about 90%, at least about 2-fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14, at least fold about 15 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 250 fold, at least about 300 fold, at least about 350 fold, at least about 400 fold, at least about 500 fold, at least about 600 fold, at least about 700 fold, at least about 800 fold, at least about 900 fold, at least about 1000 fold, at least about 1500 fold, at least about 2000 fold, or at least about 3000 fold.

In some embodiments, the systems and methods as disclosed herein can modulate (e.g., increase or decrease) expression of a target gene (e.g., upon introducing a complex comprising the heterologous polypeptide into a cell or population of cells) by at most about 50%, at most about 60%, at most about 70%, at most about 80%, at most about 90%, at most about 2-fold, at most about 3 fold, at most about 4 fold, at most about 5 fold, at most about 6 fold, at most about 7 fold, at most about 8 fold, at most about 9 fold, at most about 10 fold, at most about 11 fold, at most about 12 fold, at most about 13 fold, at most about 14, at most fold about 15 fold, at most about 20 fold, at most about 30 fold, at most about 40 fold, at most about 50 fold, at most about 60 fold, at most about 70 fold, at most about 80 fold, at most about 90 fold, at most about 100 fold, at most about 150 fold, at most about 200 fold, at most about 250 fold, at most about 300 fold, at most about 350 fold, at most about 400 fold, at most about 500 fold, at most about 600 fold, at most about 700 fold, at most about 800 fold, at most about 900 fold, at most about 1000 fold, at most about 1500 fold, at most about 2000 fold, at most about 3000 fold, at most about 5000 fold, or at most about 10000 fold.

In some embodiments, the systems and methods as disclosed herein can modulate (e.g., increase or decrease) expression of a target gene (e.g., upon introducing a complex comprising the heterologous polypeptide into a cell or population of cells) by about 10%, about 20%, about 30%, about 40%, about 50%, about 60%, about 70%, about 80%, about 90%, about 2-fold, about 3 fold, about 4 fold, about 5 fold, about 6 fold, about 7 fold, about 8 fold, about 9 fold, about 10 fold, about 11 fold, about 12 fold, about 13 fold, about 14, about 15 fold, about 20 fold, about 30 fold, about 40 fold, about 50 fold, about 60 fold, about 70 fold, about 80 fold, about 90 fold, about 100 fold, about 150 fold, about 200 fold, about 250 fold, about 300 fold, about 350 fold, about 400 fold, about 500 fold, about 600 fold, about 700 fold, about 800 fold, about 900 fold, about 1000 fold, about 1500 fold, about 2000 fold, about 3000 fold, about 5000 fold, or about 10000 fold.

In some embodiments, the systems and methods as disclosed herein can modulate (e.g., increase or decrease) expression of a target gene (e.g., upon introducing a complex comprising the heterologous polypeptide into a cell or population of cells) from below a limit of detection to a detectable level.

In some embodiments, the degree in change of expression is relative to before introducing the system of the present disclosure (e.g., a complex comprising the heterologous polypeptide) into the cell or population of cells. In some embodiments, the degree in change of expression is relative to a corresponding control cell or population of cells that are not treated with the system of the present disclosure. In some embodiments, the degree in change of expression is relative to a corresponding control cell or population of cells that are treated with an alternative to the system of the present disclosure.

In some embodiments, the systems and methods as disclosed herein can modulate (e.g., increase or decrease) an activity level of a target gene (e.g., upon introducing a complex comprising the heterologous polypeptide into a cell or population of cells). An activity level can be determined by a suitable functional assay for the target gene in question depending on the functional characteristics of the target gene. For example, an activity level of a target gene that is a mitogen could be determined by measuring cell proliferation; an activity level of a target gene that induces apoptosis could be measured by an annexin V assay or other suitable cell death assay; an activity level of an anti-inflammatory cytokine could be measured by an LPS-induced cytokine release assay.

In some embodiments, the systems and methods as disclosed herein can modulate (e.g., increase or decrease) the activity of the target gene by at least about 10%, at least about 20%, at least about 30%, at least about 40%, at least about 50%, at least about 60%, at least about 70%, at least about 80%, at least about 90%, at least about 2-fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14, at least about 15 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 250 fold, at least about 300 fold, at least about 350 fold, at least about 400 fold, at least about 500 fold, at least about 600 fold, at least about 700 fold, at least about 800 fold, at least about 900 fold, at least about 1000 fold, at least about 1500 fold, at least about 2000 fold, or at least about 3000 fold.

In some embodiments, the systems and methods as disclosed herein can modulate (e.g., increase or decrease) the activity of the target gene by at most 50%, at most 60%, at most 70%, at most 80%, at most 90%, at most about 2-fold, at most about 3 fold, at most about 4 fold, at most about 5 fold, at most about 6 fold, at most about 7 fold, at most about 8 fold, at most about 9 fold, at most about 10 fold, at most about 11 fold, at most about 12 fold, at most about 13 fold, at most about 14, at most about 15 fold, at most about 20 fold, at most about 30 fold, at most about 40 fold, at most about 50 fold, at most about 60 fold, at most about 70 fold, at most about 80 fold, at most about 90 fold, at most about 100 fold, at most about 150 fold, at most about 200 fold, at most about 250 fold, at most about 300 fold, at most about 350 fold, at most about 400 fold, at most about 500 fold, at most about 600 fold, at most about 700 fold, at most about 800 fold, at most about 900 fold, at most about 1000 fold, at most about 1500 fold, at most about 2000 fold, at most about 3000 fold, at most about 5000 fold, or at most about 10000 fold.

In some embodiments, the systems and methods as disclosed herein can modulate (e.g., increase or decrease) the activity of the target gene by about 10%, about 20%, about 30%, about 40%, about 50%, about 60%, about 70%, about 80%, about 90%, about 2-fold, about 3 fold, about 4 fold, about 5 fold, about 6 fold, about 7 fold, about 8 fold, about 9 fold, about 10 fold, about 11 fold, about 12 fold, about 13 fold, about 14, about 15 fold, about 20 fold, about 30 fold, about 40 fold, about 50 fold, about 60 fold, about 70 fold, about 80 fold, about 90 fold, about 100 fold, about 150 fold, about 200 fold, about 250 fold, about 300 fold, about 350 fold, about 400 fold, about 500 fold, about 600 fold, about 700 fold, about 800 fold, about 900 fold, about 1000 fold, about 1500 fold, about 2000 fold, about 3000 fold, about 5000 fold, or about 10000 fold.

In some embodiments, the systems and methods as disclosed herein can modulate (e.g., increase or decrease) expression of a target gene (e.g., upon introducing a complex comprising the heterologous polypeptide into a cell or population of cells) from below a limit of detection to a detectable level.

In some embodiments, the degree in change of an activity level is relative to before introducing the system of the present disclosure (e.g., a complex comprising the heterologous polypeptide) into the cell or population of cells. In some embodiments, the degree in change of an activity level is relative to a corresponding control cell or population of cells that are not treated with the system of the present disclosure. In some embodiments, the degree in change of an activity level is relative to a corresponding control cell or population of cells that are treated with an alternative to the system of the present disclosure.

The systems and methods of the present disclosure can, in some cases, elicit changes in expression and/or activity level of a target gene (e.g., target endogenous gene) that persists for longer than can be achieved with alternative compositions and methods (e.g., suppression via RNAi, e.g., using siRNA). In some embodiments, persistent modulation of gene expression is advantageous as compared to transient modulation.

In some embodiments, the systems and methods as disclosed herein can modulate (e.g., increase or decrease) expression and/or activity level of a target gene for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 18 hours, at least about 20 hours, at least about 1 day, at least about 2 days, at least about 3 days, at least about 4 days, at least about 5 days, at least about 6 days, at least about 7 days, at least about 8 days, at least about 9 days, at least about 10 days, at least about 14 days, at least about 21 days, at least about 28 days, at least about 5 weeks, at least about 6 weeks, at least about 7 weeks, at least about 8 weeks, at least about 9 weeks, at least about 10 weeks, at least about 12 weeks, at least about 14 weeks, at least about 18 weeks, at least about 20 weeks, at least about 26 weeks, or at least about 5 months, at least about 6 months, at least about 9 months, at least about 12 months, or more.

In some embodiments the systems and methods as disclosed herein can modulate (e.g., increase or decrease) expression and/or activity level of a target gene (e.g., target endogenous gene) to above a certain threshold for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 14 hours, at most about 18 hours, at most about 20 hours, at most about 1 day, at most about 2 days, at most about 3 days, at most about 4 days, at most about 5 days, at most about 6 days, at most about 7 days, at most about 8 days, at most about 9 days, at most about 10 days, at most about 14 days, at most about 21 days, at most about 28 days, at most about 5 weeks, at most about 6 weeks, at most about 7 weeks, at most about 8 weeks, at most about 9 weeks, at most about 10 weeks, at most about 12 weeks, at most about 14 weeks, at most about 18 weeks, at most about 20 weeks, at most about 26 weeks, or at most about 5 months, at most about 6 months, at most about 9 months, at most about 12 months, or more.

In some embodiments, the systems and methods as disclosed herein can modulate (e.g., increase or decrease) expression and/or activity level of a target gene (e.g., target endogenous gene) to above a certain threshold for about 1 hour, about 2 hours, about 3 hours, about 4 hours, about 5 hours, about 6 hours, about 7 hours, about 8 hours, about 9 hours, about 10 hours, about 12 hours, about 14 hours, about 18 hours, about 20 hours, about 1 day, about 2 days, about 3 days, about 4 days, about 5 days, about 6 days, about 7 days, about 8 days, about 9 days, about 10 days, about 14 days, about 21 days, about 28 days, about 5 weeks, about 6 weeks, about 7 weeks, about 8 weeks, about 9 weeks, about 10 weeks, about 12 weeks, about 14 weeks, about 18 weeks, about 20 weeks, about 26 weeks, about 5 months, about 6 months, about 9 months, or about 12 months.

In some embodiments, the target gene (e.g., endogenous target gene) can be a disease-causing allele, such as a mutant variant of a wild type allele. The disease can be a genetic disease, such as a hereditary disorder. Non-limiting examples of the genetic disorder can include Duchenne muscular dystrophy (DMD), hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease. In some cases, the target gene can be a gene encoding a protein. In some cases, the target gene can be a gene regulatory sequence (e.g., promoters, enhancers, repressors, silencers, insulators, cis-regulatory elements, trans-regulatory elements, epigenetic modification (e.g., DNA methylation) sites, etc.) that can influence expression of a gene encoding a protein of interest as provided herein. For example, target gene regulatory sequences can be physically located outside of the transcriptional unit or open reading frame that encodes a product of the target gene.

In some embodiments, a target gene regulatory sequence does not contain a nucleotide sequence that is exogenous to the subject or host cell. In some embodiments, a target gene regulatory sequence does not contain an engineered or artificially generated or introduced nucleotide sequence.

In some embodiments, a target gene (e.g., target endogenous gene) is a gene that is over-expressed or under-expressed in a disease or condition. In some embodiments, a target gene is a gene that is over-expressed or under-expressed in a heritable genetic disease. In some embodiments, the disease is retinitis pigmentosa 4 (RP4).

### Heterologous Polynucleotide

In some embodiments, a target gene (e.g., an endogenous target gene) can be a disease causing gene (e.g., a mutant allele), and the systems and compositions of the present disclosure can further comprise a heterologous polynucleotide encoding a non-disease causing gene thereof (e.g., a wild type allele), e.g., as a gene replacement therapy. Accordingly, the methods as disclosed herein can comprise introducing such system or compositions to a cell or to a subject, e.g., contacting the cell with such systems or compositions (e.g., via delivery or expression of such systems or compositions in the cell).

Thus, the systems and compositions can comprise the non-disease causing wild type or variant of the target gene, as abovementioned. Alternatively or in addition to, the systems and compositions can comprise a heterologous polynucleotide sequence encoding (or comprising) at least the non-disease causing wild type or variant of the target gene (e.g., that of the endogenous target gene) as disclosed herein.

### Composition

In some aspects, the present disclosure provides a composition comprising at least a portion of the system as described, e.g., (i) the heterologous polypeptide comprising the actuator moiety or a heterologous polynucleotide encoding the heterologous polypeptide. (ii) the guide nucleic acid or a heterologous polynucleotide encoding the guide nucleic acid, as disclosed herein, (iii) the heterologous polynucleotide encoding a non-disease causing allele of a gene, for use in any of the methods as disclosed herein. The subject composition can be usable for modifying a cell in vitro, ex vivo, or in vivo. The subject composition can be usable for treating or enhancing a condition of a subject, as disclosed herein.

The composition as disclosed herein can comprise an active ingredient (e.g., the heterologous polypeptide comprising the actuator moiety, the guide nucleic acid, the heterologous polynucleotide encoding the non-disease causing allele of a gene, etc.) and optionally an additional ingredient (e.g., excipient). If necessary and/or desirable, the composition can be divided, shaped and/or packaged into a desired single- or multi-dose unit or single- or multi-implantation unit.

In some embodiments, the composition can comprise one or more heterologous polynucleotides encoding the active ingredients as disclosed herein. When there are different members within the active ingredients, each member can be encoded by a different heterologous polynucleotide. Alternatively, two or more (e.g., all of) the ingredients can be encoded by a single heterologous polynucleotide. In some cases, a single heterologous polynucleotide an encode (i) the heterologous polypeptide comprising the actuator moiety (e.g., dCas-transcriptional effector fusion protein, such as dCas-KRAB or dCas-DNMT) and (ii) one or more guide nucleic acids (e.g., at least 1, at least 2, at least 3, at least 4, at least 5, or more guide nucleic acids) for targeting specific region(s) or sequence(s) of the target gene. In some cases, a single heterologous polynucleotide an encode (i) the heterologous polypeptide comprising the actuator moiety (e.g., dCas-transcriptional effector fusion protein, such as dCas-KRAB or dCas-DNMT), (ii) one or more guide nucleic acids (e.g., at least 1, at least 2, at least 3, at least 4, at least 5, or more guide nucleic acids) for targeting specific region(s) or sequence(s) of the target gene, and (iii) the heterologous polynucleotide encoding a non-disease causing allele of a gene.

The one or more heterologous polynucleotides can further comprise one or more promoters (or one or more transcriptional control elements, as used interchangeably herein). Different active ingredients encoded by the one or more heterologous polynucleotides can be under the control of the same promoter or different promoters. A promoter as disclosed herein can be active in a eukaryotic, mammalian, non-human mammalian or human cell. The promoter can be an inducible or constitutively active promoter. Alternatively or additionally, the promoter can be tissue or cell specific. Non-limiting examples of suitable eukaryotic promoters (i.e. promoters functional in a eukaryotic cell) can include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter can be a fungi promoter. The promoter can be a plant promoter. A database of plant promoters can be found (e.g., PlantProm). The expression vector may also contain a ribosome binding site for translation initiation and a transcription terminator. The expression vector may also include appropriate sequences for amplifying expression. In some cases, a promoter as disclosed herein can be a promoter specific for any of the tissues provided herein, or a promoter specific for any of the cell types provided herein.

A heterologous polynucleotide of the one or more heterologous polynucleotides (e.g., the single heterologous polynucleotide) can have a size of at least or up to about 2.5 kilobases, at least or up to about 2.6 kilobases, at least or up to about 2.7 kilobases, at least or up to about 2.8 kilobases, at least or up to about 2.9 kilobases, at least or up to about 3.0 kilobases, at least or up to about 3.1 kilobases, at least or up to about 3.2 kilobases, at least or up to about 3.3 kilobases, at least or up to about 3.4 kilobases, at least or up to about 3.5 kilobases, at least or up to about 3.6 kilobases, at least or up to about 3.7 kilobases, at least or up to about 3.8 kilobases, at least or up to about 3.9 kilobases, at least or up to about 4.0 kilobases, at least or up to about 4.1 kilobases, at least or up to about 4.2 kilobases, at least or up to about 4.3 kilobases, at least or up to about 4.4 kilobases, at least or up to about 4.5 kilobases, at least or up to about 4.6 kilobases, at least or up to about 4.7 kilobases, at least or up to about 4.8 kilobases, at least or up to about 4.9 kilobases, at least or up to about 5.0 kilobases, at least or up to about 5.5 kilobases, at least or up to about 6.0 kilobases, at least or up to about 6.5 kilobases, at least or up to about 7.0 kilobases, at least or up to about 7.5 kilobases, at least or up to about 8.0 kilobases, at least or up to about 9.0 kilobases, or at least or up to about 10 kilobases. In some cases, the heterologous polynucleotide of the one or more heterologous polynucleotides (e.g., the single heterologous polynucleotide) can have a size of between about 3 kilobases and about 5 kilobases, between about 3 kilobases and about 4.8 kilobases, between about 3 kilobases and about 4.6 kilobases, between about 3 kilobases and about 4.4 kilobases, between about 3 kilobases and about 4.2 kilobases, between about 3 kilobases and about 4.0 kilobases, between about 3 kilobases and about 3.5 kilobases, between about 3.5 kilobases and about 5 kilobases, between about 3.5 kilobases and about 4.8 kilobases, between about 3.5 kilobases and about 4.6 kilobases, between about 3.5 kilobases and about 4.4 kilobases, between about 3.5 kilobases and about 4.2 kilobases, between about 3.5 kilobases and about 4 kilobases, between about 4 kilobases and about 5 kilobases, between about 4 kilobases and about 4.9 kilobases, between about 4 kilobases and about 4.8 kilobases, between about 4 kilobases and about 4.7 kilobases, between about 4 kilobases and about 4.6 kilobases, between about 4 kilobases and about 4.5 kilobases, between about 4 kilobases and about 4.4 kilobases, between about 4 kilobases and about 4.3 kilobases, between about 4 kilobases and about 4.2 kilobases, or between about 4 kilobases and about 4.1 kilobases.

A method of delivery of the one or more heterologous polynucleotides provided herein to the cell can involve viral delivery methods or non-viral delivery methods. Thus, the one or more heterologous polynucleotides can be one or more viral vectors (e.g., one or more AAV vectors). Alternatively, the one or more heterologous polynucleotides can be non-viral vectors that are complexed with or encapsulated by non-viral delivery moieties, such as cationic lipids and/or lipid particles (e.g., lipid nanoparticles (LNP)).

Methods of non-viral delivery of nucleic acids can include lipofection, nucleofection, microinjection, biolistics, virosomes, liposomes, immunoliposomes, polycation or lipid: nucleic acid conjugates, naked DNA, artificial virions, and agent-enhanced uptake of DNA. Cationic and neutral lipids that are suitable for efficient receptor-recognition lipofection of polynucleotides can be used. Delivery can be to cells (e.g. in vitro or ex vivo administration) or target tissues (e.g. in vivo administration).

In some embodiments, the compositions and systems provided herein are delivered to a subject using a viral vector. In some cases, the viral vector is an adeno-associated viral (AAV) vector. The term “AAV” is an abbreviation for adeno-associated virus, and may be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle. In some cases, the AAV vector is selected based on the tropism of viral vector. In some embodiments, an AAV vector with tropism for the target tissue (e.g., eye) may be used (e.g., AAV7, AAV8, AAV9) to deliver polynucleotides encoding the compositions and systems provided herein to the target tissue (e.g., eye).

RNA or DNA viral based systems can be used to target specific cells in the body and trafficking the viral payload to the nucleus of the cell. Viral vectors can be administered directly (in vivo), or they can be used to treat cells in vitro, and the modified cells can optionally be administered (ex vivo). Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted transgene. High transduction efficiencies can be observed in many different cell types and target tissues.

The tropism of a retrovirus can be altered by incorporating foreign envelope proteins, expanding the potential target population of target cells. Lentiviral vectors are retroviral vectors that can transduce or infect non-dividing cells and produce high viral titers. Selection of a retroviral gene transfer system can depend on the target tissue. Retroviral vectors can comprise cis-acting long terminal repeats with packaging capacity for up to 6-10 kb of foreign sequence. The minimum cis-acting LTRs can be sufficient for replication and packaging of the vectors, which can be used to integrate the therapeutic gene into the target cell to provide permanent transgene expression. Retroviral vectors can include those based upon murine leukemia virus (MuLV), gibbon ape leukemia virus (GaLV), Simian Immuno deficiency virus (SIV), human immuno deficiency virus (HIV), and combinations thereof.

An adenoviral-based systems can be used. Adenoviral-based systems can lead to transient expression of the transgene. Adenoviral based vectors can have high transduction efficiency in cells and may not require cell division. High titer and levels of expression can be obtained with adenoviral based vectors. Adeno-associated virus (“AAV”) vectors can be used to transduce cells with target nucleic acids, e.g., in the in vitro production of nucleic acids and peptides, and for in vivo and ex vivo gene therapy procedures.

Packaging cells can be used to form virus particles capable of infecting a host cell. Such cells can include 293 cells, (e.g., for packaging adenovirus), and Psi2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. Viral DNA can be packaged in a cell line, which can contain a helper plasmid encoding the other AAV genes, namely rep and cap, while lacking ITR sequences. The cell line can also be infected with adenovirus as a helper. The helper virus can promote replication of the AAV vector and expression of AAV genes from the helper plasmid. Contamination with adenovirus can be reduced by, e.g., heat treatment to which adenovirus is more sensitive than AAV.

A host cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein is used to establish a new cell line comprising one or more vector-derived sequences. In some embodiments, a cell transiently transfected with the compositions of the disclosure (such as by transient transfection of one or more vectors, or transfection with RNA), and modified through the activity of an actuator moiety such as a CRISPR complex, is used to establish a new cell line comprising cells containing the modification but lacking any other exogenous sequence.

Any suitable vector compatible with the host cell can be used with the methods of the disclosure. Non-limiting examples of vectors for eukaryotic host cells include pXT1, pSG5 (Stratagene™), pSVK3, pBPV, pMSG, and pSVLSV40 (Pharmacia™).

In some embodiments, the additional ingredient of the composition as disclosed herein can comprise an excipient. Non-limiting examples of the excipient can include solvents, dispersion media, diluents, or other liquid vehicles, dispersion or suspension aids, surface active agents, isotonic agents, thickening or emulsifying agents, preservatives, lipidoids, liposomes, lipid nanoparticles, polymers, lipoplexes, core-shell nanoparticles, peptides, proteins, hyaluronidase, nanoparticle mimics, inert diluents, buffering agents, lubricating agents, oils, and combinations thereof. In some examples, the composition as disclosed herein can include one or more excipients, each in an amount that together increases the stability of (i) the heterologous polypeptide or the heterologous gene encoding thereof and/or (ii) cells or modified cells.

In some aspects, the present disclosure provides a kit comprising such composition and instructions directing (i) contacting the cell with the composition (e.g., in vitro, ex vivo, or in vivo), or (ii) administration of cells comprising any one of the compositions disclosed herein to a subject. The subject may have or may be suspected of having a condition, such as a hereditary disease.

In some embodiments, any of the compositions as disclosed herein, can be administered to the subject via orally, intraperitoneally, intravenously, intraarterially, transdermally, intramuscularly, liposomally, via local delivery by catheter or stent, subcutaneously, intraadiposally, or intrathecally. In particular aspects, the compositions and systems provided herein (including polynucleotides encoding said compositions and systems, e.g., contained in an AAV vector) can be administered to a subject via intravitreal or subretinal injection administration.

The compositions (e.g., pharmaceutical compositions) as disclosed herein can be suitable for administration to humans. In addition, such compositions can be suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

### Cells

In some embodiments, a cell as provided herein may be referred to as a target cell. In some embodiments, the systems, compositions, and methods as provided herein can be applied to modify a target cell (e.g., modify expression profile of a target gene of the target cell). A target cell can include a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and a apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy.

Non-limiting examples of cells which can be target cells include, but are not limited to, lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

The cell (or target cell) can be engineered to comprise (or exhibit) any one of the systems or compositions as disclosed herein or can be treated by any one of the methods disclosed herein in vitro or ex vivo, then administered to the subject, e.g., to treat a condition of the subject. For example, any subject modified cell product can be administered to the subject to treat a condition of a bodily tissue of the subject. In some cases, the cell can be resident inside the subject's body, and any of the systems or compositions thereof can be administered to the subject, to contact the cell by the systems/compositions (e.g., to engineer the cell with the systems/compositions).

### EXAMPLES

### Example 1. Gene Expression Modulation

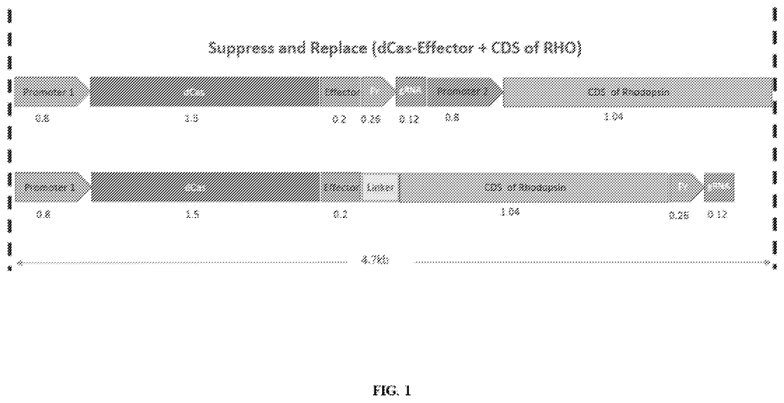

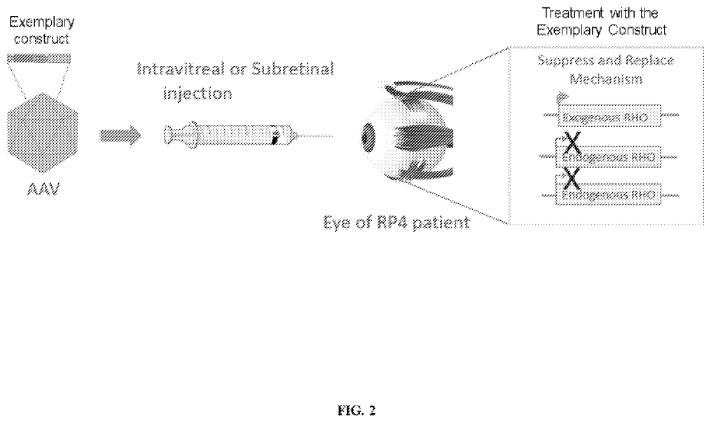

Gene expression can be modulated in a cell by utilizing a system or a method described herein. In some cases, the gene being modulated by the system, or the method can be a mutant allele that can cause a disease or condition in a subject. In some cases, the gene being modulated can be a non-disease causing variant (e.g. a wild type allele). In some embodiments, the gene expression can be modulated by the system, or the method described herein by both decreasing the expression of the mutant allele in a cell and simultaneously increasing expression of the wild type allele. In some cases, the wild type allele is encoded by at least one of the heterologous polynucleotides described herein. FIG. 1 illustrates exemplary constructs encoding the dCas, the actuator moiety (effector), and coding sequence (CDS) of an endogenous target gene. dCas can be coupled with a transcription repressor for decreasing expression of the expression of the mutant allele of the endogenous target gene in the cell, while the CDS can encode the wild type allele of the endogenous target gene for increasing the expression of the wild type allele of the endogenous target gene in the same cell. The top construct of FIG. 1 illustrates that the dCas and the CDS can be under the control of different promoters (e.g., different constitutive promoters; different tissue specific promoters; a tissue specific promoter and a constitutive promoter respectively; a constitutive promoter and a tissue specific promoter respectively, etc.). The bottom construct of FIG. 1 illustrates that the dCas and the CDS can be under the control of the same promoter (e.g., any one of the promoter described herein). FIG. 2 illustrates a schematic for treating retinitis pigmentosa with the system described herein. AAV can be engineered to deliver an exemplary construct via subretinal or intravitreal injection to a subject in need thereof, where the expression of the exemplary construct can simultaneously decrease expression of endogenous mutated Rhodopsin and increase express of Rhodopsin encoded by the heterologous CDS of the construct.

The modulation of the endogenous target gene expression by the system or method described herein can be used to treat a disease or condition in a subject. A subject suspected of having a disease or condition associated with mutation of the endogenous target gene can be first screened for the presence of a mutant allele of the endogenous target gene. Afterwards, the system described herein can be administered to the subject to simultaneously decrease expression of the mutant allele of endogenous target gene and increase expression of the non-disease causing allele of endogenous target gene.

### Example 2. Rhodopsin Expression Modulation

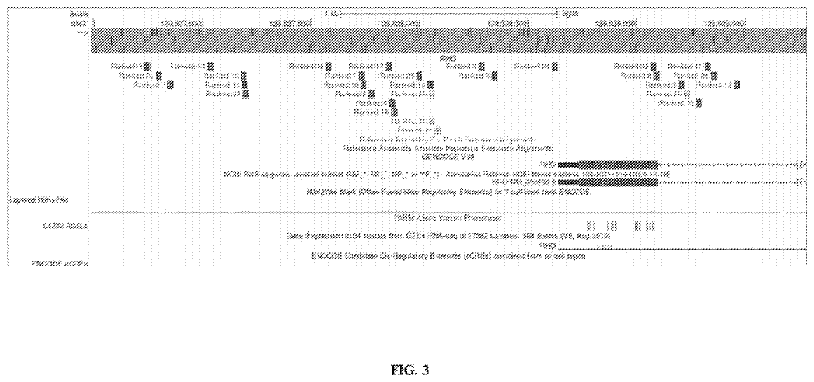

Rhodopsin expression can be modulated in a cell by utilizing a system or a method described herein. In some cases, the Rhodopsin is a mutant allele of Rhodopsin. In some cases, the mutant Rhodopsin can cause disease in a subject. In some cases, the Rhodopsin is a non-disease causing variant of Rhodopsin (e.g. wild type allele of Rhodopsin). In some embodiments, the Rhodopsin expression can be modulated by the system, or the method described herein by both decreasing the expression of the mutant allele of Rhodopsin in a cell and simultaneously increasing expression of the wild type allele of Rhodopsin. In some cases, the wild type allele of Rhodopsin is encoded by at least one of the heterologous polynucleotides described herein. FIG. 1 illustrates exemplary constructs encoding the dCas, the actuator moiety (effector), and coding sequence (CDS) of an endogenous target gene. dCas can be coupled with a transcription repressor for decreasing expression of the expression of the mutant allele of Rhodopsin in the cell, while the CDS can encode the wild type allele of Rhodopsin for increasing the expression of the wild type allele of Rhodopsin in the same cell. The top construct of FIG. 1 illustrates that the dCas and the CDS can be under the control of different promoters (e.g., different constitutive promoters; different tissue specific promoters; a tissue specific promoter and a constitutive promoter respectively; a constitutive promoter and a tissue specific promoter respectively, etc.). The bottom construct of FIG. 1 illustrates that the dCas and the CDS can be under the control of the same promoter (e.g., any one of the promoter described herein). FIG. 3 illustrates exemplary transcripts that can be targeted by the gRNA of the system and the method described herein for decreasing or increasing the expression of Rhodopsin. The modulation of the Rhodopsin expression by the system or method described herein can be used to treat a disease or condition in a subject. A subject suspected of having a disease or condition associated with Rhodopsin mutation can be first screened for the presence of Rhodopsin variant (e.g., a mutant allele of Rhodopsin). After, the system described herein can be administered to the subject to simultaneously decrease expression of the mutant allele of Rhodopsin and increase expression of the non-disease causing allele of Rhodopsin (encoded from the heterologous polynucleotide described herein).

### Example 3. Expression Cassettes (or Constructs)

The systems as provided herein can be delivered to a cell via one or more expression cassettes encoding one or more components of the systems. The one or more expression cassettes can comprise a vector, such as a viral vector (e.g., an AAV vector comprising two Inverted terminal repeats (ITRs)).

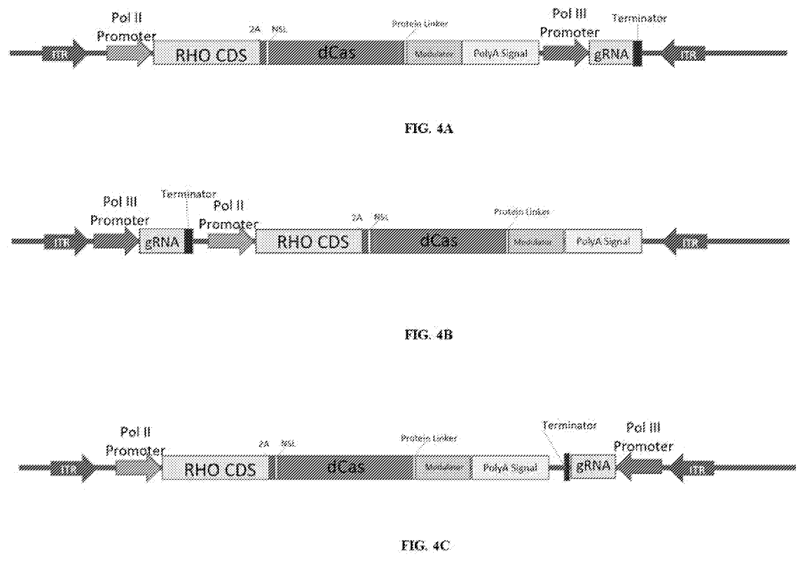

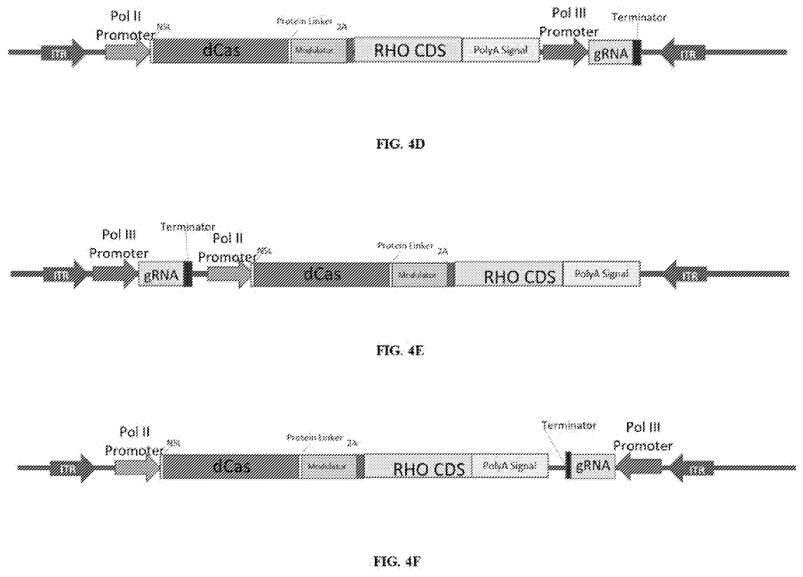

FIGS. 4A-4F schematically illustrate examples of a single vector encoding one or more components the system as provided herein.

FIG. 4A schematically illustrates a construct comprising an RNA Pol II promoter driving the expression of DNA sequences encoding Rhodopsin (RHO), a deactivated Cas (dCas) comprising a self-cleaving 2A sequence and a nuclear localization signal located at the 5′-end of the dCas, a modulator comprising a protein linker (e.g., a GS linker) located at the 5′-end of the modulator, and a poly(A) signal. The construct further comprises an RNA Pol III promoter driving the expression of a gRNA scaffold-spacer-terminator sequence, located downstream of the poly(A) signal.

FIG. 4B schematically illustrates a construct comprising an RNA Pol III promoter driving the expression of a gRNA scaffold-spacer-terminator sequence. The construct further comprises an RNA Pol II promoter located downstream of the RNA Pol III promoter. The RNA Pol II promoter drives the expression of DNA sequences encoding RHO, a deactivated Cas (dCas) comprising a self-cleaving 2A sequence, and a nuclear localization signal located at the 5′-end of the dCas, a modulator comprising a protein linker located at the 5′-end of the modulator, and a poly(A) signal.

FIG. 4C schematically illustrates a construct comprising an RNA Pol II promoter driving the expression of DNA sequences encoding Rhodopsin (RHO), a deactivated Cas (dCas) comprising a self-cleaving 2A sequence and a nuclear localization signal located at the 5′-end of the dCas, a modulator comprising a linker located at the 5′-end of the modulator, and a poly(A) signal. The construct further comprises a reverse-oriented RNA Pol III promoter driving the expression of a gRNA scaffold-spacer-terminator sequence, located downstream of the poly(A) signal.

FIG. 4D schematically illustrates a construct comprising an RNA Pol II promoter driving the expression of DNA sequences encoding a deactivated Cas (dCas) comprising a nuclear localization signal located at the 5′-end of the dCas, a modulator comprising a protein linker at the 5′-end of the modulator, a self-cleaving 2A sequence, RHO, and a poly(A) signal. The construct further comprises an RNA Pol III promoter driving the expression of a gRNA scaffold-spacer-terminator sequence, located downstream of the poly(A) signal.

FIG. 4E schematically illustrates a construct comprising an RNA Pol III promoter driving the expression of a gRNA scaffold-spacer-terminator sequence. The construct further comprises an RNA Pol II promoter located downstream of the RNA Pol III promoter. The RNA Pol II promoter drives the expression of DNA sequences encoding a deactivated Cas (dCas) comprising a nuclear localization signal located at the 5′-end of the dCas, a modulator comprising a protein linker at the 5′-end of the modulator, a self-cleaving 2A sequence, RHO, and a poly(A) signal.

FIG. 4F schematically illustrates a construct comprising an RNA Pol II promoter driving the expression of DNA sequences encoding a deactivated Cas (dCas) comprising a nuclear localization signal located at the 5′-end of the dCas, a modulator comprising a protein linker at the 5′-end of the modulator, a self-cleaving 2A sequence, RHO, and a poly(A) signal. The construct further comprises a reverse-oriented RNA Pol III promoter driving the expression of a gRNA scaffold-spacer-terminator sequence, located downstream of the poly(A) signal.

### Example 4. Rhodopsin Modulation in Human Retina Tissue & 3D Organoid

The systems and methods described can simultaneously suppress endogenous Rhodopsin expression in a cell, targeting either a mutant or non-disease-causing variant, while increasing expression of exogenous Rhodopsin.

### A. Suppression of an Endogenous Target Gene and an Activation of Exogenous Target Gene in Human Retina Tissue

A study was conducted to assess endogenous and exogenous Rhodopsin expression in human retina tissue using an exemplary construct described herein.

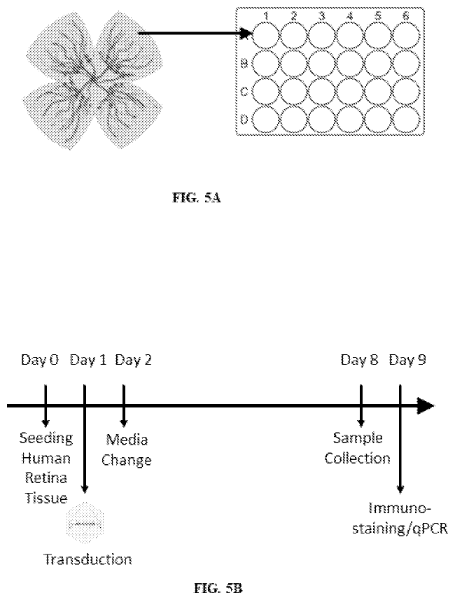

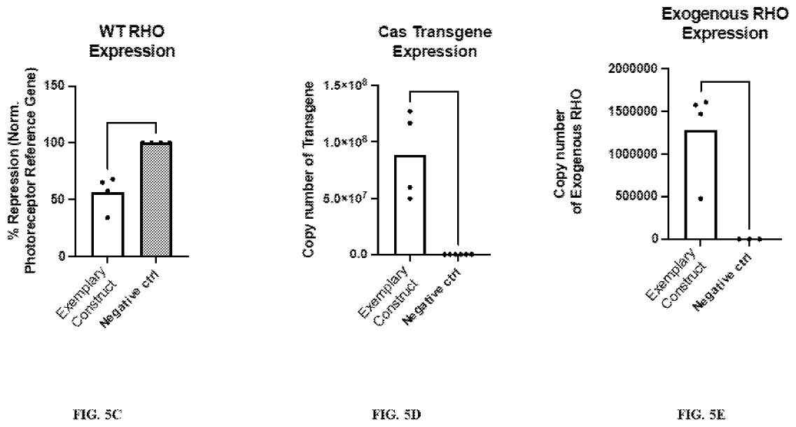

As depicted in FIGS. 5A and 5B, human post-mortem donor eyes were obtained by San Diego Eye Bank. The human retina was isolated, and 3 mm biopsy punches were collected and transferred in a 24-well tissue culture plate with culture media (DMEM/F12+10% fetal bovine serum+1% 1-glutamine+1% penicillin/streptomycin). Retina tissues were incubated at 37° C., 5% CO₂ for 24 h prior to AAV transduction. On the day of transduction, media containing AAV (e.g., exemplary constructs as described in Example 3) was added to retinal punctures at 1.0×10¹¹ vg per 3 mm biopsy puncture. After 24 hrs. transduction, the tissues were washed with PBS and replenished with complete culture media for 8 days, and medium was changed every 3 days. Tissues were collected and processed for either histology or molecular analysis. For histology, tissue was fixed in 4% PFA. For molecular analysis, processed for qRT-PCR.

Referring to FIG. 5C, plotted data illustrates that endogenous Rhodopsin expression was reduced by approximately 50% in the exemplary construct treated tissue, as compared to the negative control tissue (isolated human retina tissue without AAV treatment), after normalization to the photoreceptor reference gene.

FIG. 5D illustrates that the copy number of the Cas transgene in the exemplary construct treated tissue was approximately 0.88×108, whereas it was undetectable in the negative control tissue. Similarly, FIG. 5E demonstrates that the copy number of exogenous Rhodopsin expression in the exemplary construct treated tissue was about 1,200,000, whereas it was not detected in the negative control tissue.

### B. Suppression of an Endogenous Target Gene and an Activation of Exogenous Target Gene in 3D Retinal Organoid

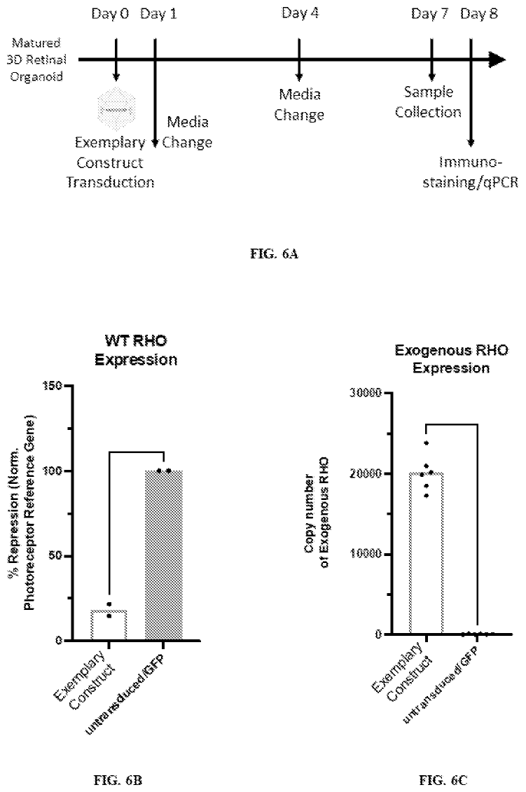

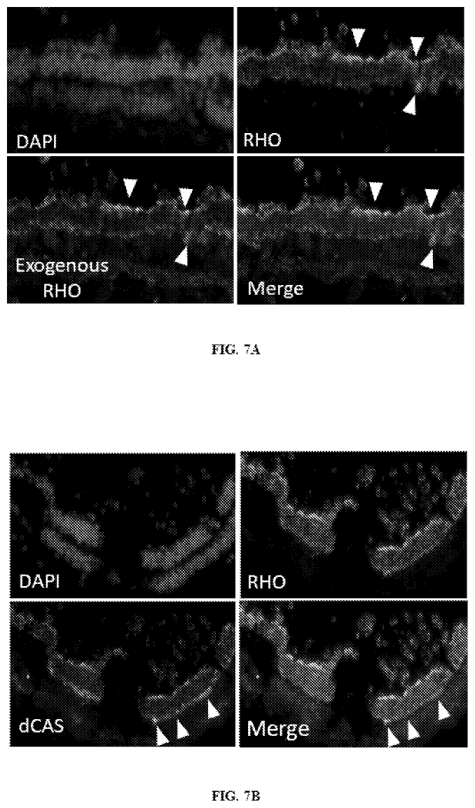

Human induced pluripotent stem cells (iPSCs) were expanded in stem cell medium, dissociated, and then subjected to 3D retinal differentiation in neural induction medium (e.g., for weeks), to generate 3D retinal organoid. Integrity of the 3D retinal organoids was confirmed by detecting presence of one or more photoreceptor cells differentiation marker (e.g., OTX2, Recoverin, and SNCG). The mature organoids were subsequently transduced with the exemplary construct, and the media was changed on Day 1 and Day 4. Samples were collected on Day 7 for immunostaining and qPCR analysis, as depicted in FIG. 6A. The plotted data in FIG. 6B shows that endogenous Rho expression was suppressed by approximately 80% in the exemplary construct treated organoids compared to untransduced control organoids. Similarly, FIG. 6C demonstrates that the copy number of exogenous Rhodopsin expression in the exemplary construct treated 3D organoids was approximately 20,000, while it was not detectable in the negative control tissue.

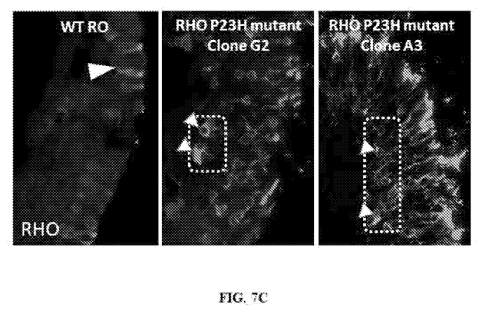

Referring to FIG. 7A and FIG. 7B, the immunostaining of human retina tissue transduced with the exemplary construct further confirmed that the exogenous RHO is express at the apical region where endogens RHO is located (FIG. 7A), and the transgene dCas-modulators were found in the RHO⁺ cells (FIG. 7B). These results collectively suggest that the exemplary construct transduction can suppress endogenous target gene expression and activate exogenous target gene expression, leading to the production of non-disease protein.

### Example 5. Assessing Functions of Photoreceptor Cells Restored Via the Exemplary Construct

Human iPSCs were first engineered with RHO P23H mutation through CRISPR with HDR by Synthego. The isogenic lines with correct mutation were validated and tested for Karyotype. The wild-type and RHO P23H mutant iPSCs cells were maintained and expanded in the conditioned culture medium. Integrity of the 3D retinal organoids was confirmed by detecting presence of one or more photoreceptor cells differentiation markers (e.g., OTX2, Recoverin, and SNCG).

Referring to FIG. 7C, the immunostaining of wild-type and P23H RHO mutant 3D retinal organoids shows the presence of RHO aggregations in the perinuclei region, suggesting ER stress in the P23H RHO mutant. To further demonstrate the efficacy of the exemplary construct in rescuing the pathological phenotype of P23H RHO mutants, we will conduct in vitro efficacy studies at two different time frames at Day 120 or Day 180. Following tests will be conducted to confirm the restoration of the photoreceptor cell function by the exemplary construct treatment.

### A. Phenotypic Measurements: Multielectrode Array (MEA)

WT and P23H RHO mutant 3D retinal organoids will be transduced with the exemplary construct on day 120 or 180 at dose of 5×10¹⁰-5×10¹¹ vg per organoid and cultured in a 5% CO₂ incubator with mild agitation. Media will be replaced 24 hours after. Organoids will be kept in 3D maturation media until Day 220 or until outer segment of retinal tissue appears. To test photoreceptors responses to light, matured 3D organoids will be sectioned in thin slices and loaded onto MEA plates. The electrophysiology is measured according to manufacturer's protocols.

### B. Phenotypic Measurements: Calcium Image

The testing retinal organoids will be infected at Day 180 with AAV inducing the expression of the calcium sensor GCaMP6s under the control of promoter EF1α. Infected organoids will be cultured for 3 to 4 weeks in BrainPhys media supplemented with 1% N2 Supplement, 100 μM taurine, 1 μM retinoic acid. Two days before calcium imaging recordings, organoids will be gradually transferred to BrainPhys media supplemented with 1% N2 Supplement, 100 μM taurine, 1 μM retinoic acid, 10 μM 9-cis-retinal, and 2 μM Albumin solution human.

Imaging will be carried out using a High Content Imager (Molecular Devices). Shortly before recording, the retinal organoids will be embedded in 1% low-melting agarose. Throughout the recording, the organoid being studied will be perfused with BrainPhys media supplemented with 1% N2 Supplement, 100 μM taurine, 1 μM retinoic acid, 10 μM 9-cis-retinal, 2 μM Albumin solution human. The perfusion solution will be flowing at a rate of 1-2 mL/minute, maintained at a temperature of 37° C. and bubbled with 95% O2/5% CO₂. GCaMP6s expressing cells were imaged at 980 nm. The imaging window will be set to 200 μm×200 μm and the z-plane will be adjusted to show a cross section of the organoid using Calcium Flux assay kit as described.

### C. Phenotypic Measurements: ER Stress Analysis

At Day 220, 3D-retinal organoids will be preserved and section into 25 μm thickness. Tissues are then immunostained with RHO antibodies to monitor the migration of RHO+ cells and RHO protein in photoreceptors. The images will be taken with ImageExpress Micro (Molecular Devices) to monitor the subcellular aggregation of RHO mutant protein at endoplasmic reticulum or perinuclei zone suggesting ER stress level.

It shall be understood that different aspects of the invention can be appreciated individually, collectively, or in combination with each other. Various aspects of the invention described herein may be applied to any of the particular applications disclosed herein. The compositions of matter disclosed herein in the composition section of the present disclosure may be utilized in the method section including methods of use and production disclosed herein, or vice versa.

TABLE 1
Non-limiting examples of a Cas protein or a derivative thereof.

| SEQ | |
|---|---|
| ID NO | Amino acid sequence of Cas protein or a derivative thereof |
| 1 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSDVCYTRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKIGEK |
| | SAWMLNLSIDVPKIDKGVDPSIIGGIDVGVKSPLVCAINNAFSRYSISDNDLFHFNKKMFA |
| | RRRILLKKNRHKRAGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENADYNAALNISNPKLKSTKEEP |
| 2 | MEVQKTVMKTLSLRILRPLYSQEIEKEIKEEKERRKQAGGTGELDGGFYKKLEKKHSEM |
| | FSFDRLNLLLNQLQREIAKVYNHAISELYIATIAQGNKSNKHYISSIVYNRAYGYFYNAYI |
| | ALGICSKVEANFRSNELLTQQSALPTAKSDNFPIVLHKQKGAEGEDGGFRISTEGSDLIFEI |
| | PIPFYEYNGENRKEPYKWVKKGGQKPVLKLILSTFRRQRNKGWAKDEGTDAEIRKVTE |
| | GKYQVSQIEINRGKKLGEHQKWFANFSIEQPIYERKPNRSIVGGLDVGIRSPLVCAINNSF |
| | SRYSVDSNDVFKFSKQVFAFRRRLLSKNSLKRKGHGAAHKLEPITEMTEKNDKFRKKIIE |
| | RWAKEVTNFFVKNQVGIVQIEDLSTMKDREDHFFNQYLRGFWPYYQMQTLIENKLKEY |
| | GIEVKRVQAKYTSQLCSNPNCRYWNNYFNFEYRKVNKFPKFKCEKCNLEISADYNAAR |
| | NLSTPDIEKFVAKATKGINLPEK |
| 3 | MIKVYRYEIVKPLDLDWKEFGTILRQLQQETRFALNKATQLAWEWMGFSSDYKDNHGE |
| | YPKSKDILGYTNVHGYAYHTIKTKAYRLNSGNLSQTIKRATDRFKAYQKEILRGDMSIPS |
| | YKRDIPLDLIKENISVNRMNHGDYIASLSLLSNPAKQEMNVKRKISVIIIVRGAGKTIMDRI |
| | LSGEYQVSASQIIHDDRKNKWYLNISYDFEPQTRVLDLNKIMGIDLGVAVAVYMAFQHT |
| | PARYKLEGGEIENFRRQVESRRISMLRQGKYAGGARGGHGRDKRIKPIEQLRDKIANFRD |
| | TTNHRYSRYIVDMAIKEGCGTIQMEDLTNIRDIGSRFLQNWTYYDLQQKIIYKAEEAGIK |
| | VIKIDPQYTSQRCSECGNIDSGNRIGQAIFKCRACGYEANADYNAARNIAIPNIDKIIAESI |
| | K |
| 4 | MEVEKIVADEKNNREKIALEKNKDKVKEACSKHLKVAAYCTTQVERNACLFCKARKLD |
| | DKFYQKLRGQFPDAVFWQEISEIFRQLQKQAAEIYNQSLIELYYEIFIKGKGIANASSVEH |
| | YLSRVCYRRAAELFKNAAIASGLRSKIKSNFRLKELKNMKSGLPTTKSDNFPIPLVKQKG |
| | GQYTGFEISNHNSDFIIKIPFGRWQVKKEIDKYRPWEKFDFEQVQKSPKPISLLLSTQRRK |
| | RNKGWSKDEGTEAEIKKVMNGDYQTSYIEVKRGSKICEKSAWMLNLSIDVPKIDKGVD |
| | PSIIGGIAVGVRSPLVCAINNAFSRYSISDNDLFHFNKKMFARRRILLKKNRHKRAGHGA |
| | KNKLKPITILTEKSERFRKKLIERWACEIADFFIKNKVGTVQMENLESMKRKEDSYFNIRL |
| | RGFWPYAEMQNKIEFKLKQYGIEIRKVAPNNTSKTCSKCGHLNNYFNFEYRKKNKFPHF |
| | KCEKCNFKENAAYNAALNISNPKLKSTKERP |
| 5 | MNNREKIALEKNKDKVKEACSKHLKVAAYCTTQVERNACLFCKARKLDDKFYQKLRG |
| | QFPDAVFWQEISEIFRQLQKQAAEIYNQSLIELYYEIFIKGKGIANASSVEHYLSRVCYRR |
| | AAELFKNAAIASGLRSKIKSNFRLKELKNMKSGLPTTKSDNFPIPLVKQKGGQYTGFEISN |
| | HNSDFIIKIPFGRWQVKKEIDKYRPWEKFDFEQVQKSPKPISLLLSTQRRKRNKGWSKDE |
| | GTEAEIKKVMNGDYQTSYIEVKRGSKICEKSAWMLNLSIDVPKIDKGVDPSIIGGIAVGV |
| | RSPLVCAINNAFSRYSISDNDLFHFNKKMFARRRILLKKNRHKRAGHGAKNKLKPITILT |
| | EKSERFRKKLIERWACEIADFFIKNKVGTVQMENLESMKRKEDSYFNIRLRGFWPYAEM |
| | QNKIEFKLKQYGIEIRKVAPNNTSKTCSKCGHLNNYFNFEYRKKNKFPHFKCEKCNFKE |
| | NAAYNAALNISNPKLKSTKERP |
| 6 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENA |
| 7 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFK |
| 8 | MAKNTITKTLKLRIVRPYNSAEVEKIVADEKNNREKIALEKNKDKVNACLFCKARKLDD |
| | KFYQKLRGQFPDAVFWQEISEIFRQLQKQAAEIYNQSLIELYYEIFIKGKGIANASSVEHY |
| | LSRVCYRRAAELFKNAAIASGLRSKIKSNFRLKELKNMKSGLPTTKSDNFPIPLVKQKGG |
| | QYTGFEISNHNSDFIIKIPFGRWQVKKEIDKYRPWEKFDFEQVQKSPKPISLLLSTQRRKR |
| | NKGWSKDEGTEAEIKKVMNGDYQTSYIEVKRGSKICEKSAWMLNLSIDVPKIDKGVDPS |
| | IIGGIAVGVRSPLVCAINNAFSRYSISDNDLFHFNKKMFARRRILLKKNRHKRAGHGAKN |
| | KLKPITILTEKSERFRKKLIERWACEIADFFIKNKVGTVQMENLESMKRKEDSYFNIRLRG |
| | FWPYAEMQNKIEFKLKQYGIEIRKVAPNNTSKTCSKCGHLNNYFNFEYRKKNKFPHFKC |
| | EKCNFKENAAYNAALNISNPKLKSTKERP |
| 9 | MAKNTITKTLKLRIVRPYNSAEVEKIVADEKNNREKIALECKARKLDDKFYQKLRGQFP |
| | DAVFWQEISEIFRQLQKQAAEIYNQSLIELYYEIFIKGKGIANASSVEHYLSRVCYRRAAE |
| | LFKNAAIASGLRSKIKSNFRLKELKNMKSGLPTTKSDNFPIPLVKQKGGQYTGFEISNHNS |
| | DFIIKIPFGRWQVKKEIDKYRPWEKFDFEQVQKSPKPISLLLSTQRRKRNKGWSKDEGTE |
| | AEIKKVMNGDYQTSYIEVKRGSKICEKSAWMLNLSIDVPKIDKGVDPSIIGGIAVGVRSPL |
| | VCAINNAFSRYSISDNDLFHFNKKMFARRRILLKKNRHKRAGHGAKNKLKPITILTEKSE |
| | RFRKKLIERWACEIADFFIKNKVGTVQMENLESMKRKEDSYFNIRLRGFWPYAEMQNKI |
| | EFKLKQYGIEIRKVAPNNTSKTCSKCGHLNNYFNFEYRKKNKFPHFKCEKCNFKENAAY |
| | NAALNISNPKLKSTKERP |
| 10 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 12 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 13 | MNMSKTTISVKLKIIDLSSEKKEFLDNYFNEYAKATTFCQLRIRRLLRNTHWLGKKEKSS |
| | KKWIFESGICDLCGENKELVNEDRNSGEPAKICKRCYNGRYGNQMIRKLFVSTKKREVQ |
| | ENMDIRRVAKLNNTHYHRIPEEAFDMIKAADTAEKRRKKNVEYDKKRQMEFIEMFNDE |
| | KKRAARPKKPNERETRYVHISKLESPSKGYTLNGIKRKIDGMGKKIERAEKGLSRKKIFG |
| | YQGNRIKLDSNWVRFDLAESEITIPSLFKEMKLRITGPTNVHSKSGQIYFAEWFERINKQP |
| | NNYCYLIRKTSSNGKYEYYLQYTYEAEVEANKEYAGCLGVDIGCSKLAAAVYYDSKNK |
| | KAQKPIEIFTNPIKKIKMRREKLIKLLSRVKVRHRRRKLMQLSKTEPIIDYTCHKTARKIVE |
| | MANTAKAFISMENLETGIKQKQQARETKKQKFYRNMFLFRKLSKLIEYKALLKGIKIVY |
| | VKPDYTSQTCSSCGADKEKTERPSQAIFRCLNPTCRYYQRDINADFNAAVNIAKKALNN |
| | TEVVTTLL |
| 14 | MPSETYITKTLSLKLIPSDEEKQALENYFITFQRAVNFAIDRIVDIRSSFRYLNKNEQFPAV |
| | CDCCGKKEKIMYVNISNKTFKFKPSRNQKDRYTKDIYTIKPNAHICKTCYSGVAGNMFIR |
| | KQMYPNDKEGWKVSRSYNIKVNAPGLTGTEYAMAIRKAISILRSFEKRRRNAERRIIEYE |
| | KSKKEYLELIDDVEKGKTNKIVVLEKEGHQRVKRYKHKNWPEKWQGISLNKAKSKVK |
| | DIEKRIKKLKEWKHPTLNRPYVELHKNNVRIVGYETVELKLGNKMYTIHFASISNLRKPF |
| | RKQKKKSIEYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMAKKLRGDKT |
| | KKIRLYHEIRKKFRHKVKYFRRNYLHNISKQIVEIAKENTPTVIVLEDLRYLRERTYRGK |
| | GRSKKAKKTNYKLNTFTYRMLIDMIKYKAEEAGVPVMIIDPRNTSRKCSKCGYVDENN |
| | RKQASFKCLKCGYSLNADLNAAVNIAKAFYECPTFRWEEKLHAYVCSEPDK |
| 15 | MKSFKLKLLPTDEQNVLLNEVFCKWASLCTRMASKGHDKERLAPPDSSGNYFNKTQLN |
| | QVNTDVTDHMGALEESASQKERAVEKVKRRLKLISDMLSEPNLRDVSQQKPTTFRPLE |
| | WVKEGLLKTKYHTVHYWQKECDKLTKQKERMEKTIEKIKKGKITFKPTKMSLHQNCFS |
| | LSFGKGTFSMRPFSDTKRGINLDMLTAPIQPAIGKNDGKSSLRSKEFIARNIENYIIFSIHSQ |
| | LFGLSRSEELLLNAKKEELVAKRDAMLKKKSDSLSKKIKELEKIVGRKITDSERSEIMSQ |
| | GGKLSSEKFSEDNSYLKTLKVLAKDIIGREELFRLKKYPIVIRKPLNERKKLKNLKPDEW |
| | EYYLQLSYDELEKKEFTPKTIMGIDRGLKHILAIAIYDPVQNKFVKNMLIPNPILGWKWK |
| | LRKIKRSIQHMERRIRAQQNAHVPENQLKKRLKSIENKIDYYYHNVSRQILNLAHDFKSA |
| | IVVEDLQNMKQHGRKKSKGLRGLNYALSNFDYGKIMGLVKYKAESENVPLLTVLPAGT |
| | SQNCAYCLLYGKEQGNYVRNNVNSKIGKCKLHGEIDADINAARTIAICYHKNINEPKPY |
| | GERKTFKRK |
| 16 | MKYTKVMRYQIIKPLNAEWDELGMVLRDIQKETRAALNKTIQLCWEYQGFSADYKQIH |
| | GQYPKPKDVLGYTSMHGYAYDRLKNEFSKIASSNLSQTIKRAVDKWNSDLKEILRGDRS |
| | IPNFRKDCPIDIVKQSTKIQKCNDGYVLSLGLINREYKNELGRKNGVFDVLIKANDKTQQ |
| | TILERIINGDYTYTASQIINHKNKWFINLTYQFETKETALDPNNVMGVDLGIVYPVYIAFN |
| | NSLHRYHIKGGEIERFRRQVEKRKRELLNQGKYCGDGRKGHGYATRTKSIESISDKIARF |
| | RDTCNHKYSRFIVDMALKHNCGIIQMEDLTGISKESTFLKNWTYYDLQQKIEYKAREAGI |
| | QVIKIEPQYTSQRCSKCGYIDKENRQEQATFKCIECGFKTNADYNAARNIAIPNIDKIIRKT |
| | LKMQ |
| 17 | MTLLVKVVKIHLISEQFDKAGNRIDYEEVNKILWELQKQTREAKNKTVQLLWEWNNFS |
| | SDYVKASGIYPKAKDIFGYSSVHGQANKELRTKLALNSSNLSTTTMDVCKNFNTYKKEV |
| | WKGKRSVPSYKSDQPLDLHKDSIKLIYENNEFYVRLALLKKAEFAKYGFKDGFRFKMQ |
| | VKDNSTKTILERCFDEVYKINASKLLYDQKKKKWKLNLSYSFDNKNISELDKEKILGVD |
| | VGVNCPLVASVFGDRDRFIIKGGEIEKFRKSVEARRRSMLEQTKYCGDGRIGHGRKKRT |
| | EPALNIGDKIARFRDTTNHKYSRALIEYAVKKGCGTIQMEKLTGITSKSDRFLKDWTYYD |
| | LQTKIENKAKEVGINVVYIAPKYTSQRCSKCGYIHKDNRPNQAKFRCLECDFESNADYN |
| | ASQNIGIKNIDKIIEKDLQKQESEVQVNENK |
| 18 | MGESVKAIKLKILDMFLDPECTKQDDNWRKDLSTMSRFCAEAGNMCLRDLYNYFSMP |
| | KEDRISSKDLYNAMYHKTKLLHPELPGKVANQIVNHAKDVWKRNAKLIYRNQISMPTY |
| | KITTAPIRLQNNIYKLIKNKNKYIIDVQLYSKEYSKDSGKGTHRYFLVAVRDSSTRMIFDR |
| | IMSKDHIDSSKSYTQGQLQIKKDHQGKWYCIIPYTFPTHETVLDPDKVMGVDLGVAKAV |
| | YWAFNSSYKRGCIDGGEIEHFRKMIRARRVSIQNQIKHSGDARKGHGRKRALKPIETLSE |
| | KEKNFRDTINHRYANRIVEAAIKQGCGTIQIENLEGIADTTGSKFLKNWPYYDLQTKIVN |
| | KAKEHGITVVAINPQYTSQRCSMCGYIEKTNRSSQAVFECKQCGYGSRTICINCRHVQVS |
| | GDVCEECGGIVKKENVNADYNAAKNISTPYIDQIIMEKCLELGIPYRSITCKECGHIQASG |
| | NTCEVCGSTNILKPKKIRKAK |
| 19 | MITVRKIKLTIMGDKDTRNSQYKWIRDEQYNQYRALNMGMTYLAVNDILYMNESGLEI |
| | RTIKDLKDCEKDIDKNKKEIEKLTARLEKEQNKKNSSSEKLDEIKYKISLVENKIEDYKLK |
| | IVELNKILEETQKERMDIQKEFKEKYVDDLYQVLDKIPFKHLDNKSLVTQRIKADIKSDK |
| | SNGLLKGERSIRNYKRNFPLMTRGRDLKFKYDDNDDIEIKWMEGIKFKVILGNRIKNSLE |
| | LRHTLHKVIEGKYKICDSSLQFDKNNNLILNLTLDIPIDIVNKKVSGRVVGVDLGLKIPAY |
| | CALNDVEYIKKSIGRIDDFLKVRTQMQSRRRRLQIAIQSAKGGKGRVNKLQALERFAEK |
| | EKNFAKTYNHFLSSNIVKFAVSNQAEQINMELLSLKETQNKSILRNWSYYQLQTMIEYK |
| | AQREGIKVKYIDPYHTSQTCSKCGNYEEGQRESQADFICKKCGYKVNADYNAARNIAM |
| | SNKYITKKEESKYYKIKESMV |
| 20 | MAKNTITKTLKLRIVRPYNSAEVEKIVADEKERRKQAGGTGELDGGFYKKLEKKHSEMF |
| | SFDRLNLLLNQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 21 | MEVQKTVMKTLSLRILRPLYSQEIEKEIKEEKERRKQAGGTGELDDKFYQKLRGQFPDA |
| | VFWQEISEIFRQLQKQAAEIYNQSLIELYYEIFIKGKGIANASSVEHYLSRVCYRRAAELF |
| | KNAAIASGLRSKIKSNFRLKELKNMKSGLPTTKSDNFPIPLVKQKGGQYTGFEISNHNSD |
| | FIIKIPFGRWQVKKEIDKYRPWEKFDFEQVQKSPKPISLLLSTQRRKRNKGWSKDEGTEA |
| | EIKKVMNGDYQTSYIEVKRGSKICEKSAWMLNLSIDVPKIDKGVDPSIIGGIAVGVRSPLV |
| | CAINNAFSRYSISDNDLFHFNKKMFARRRILLKKNRHKRAGHGAKNKLKPITILTEKSER |
| | FRKKLIERWACEIADFFIKNKVGTVQMENLESMKRKEDSYFNIRLRGFWPYAEMQNKIE |
| | FKLKQYGIEIRKVAPNNTSKTCSKCGHLNNYFNFEYRKKNKFPHFKCEKCNFKENAAYN |
| | AALNISNPKLKSTKERP |
| 22 | MAKNTITKTLKLRIVRPLYSQEIEKEIKEEKERRKQAGGTGELDDKFYQKLRGQFPDAVF |
| | WQEISEIFRQLQKQAAEIYNQSLIELYYEIFIKGKGIANASSVEHYLSRVCYRRAAELFKN |
| | AAIASGLRSKIKSNFRLKELKNMKSGLPTTKSDNFPIPLVKQKGGQYTGFEISNHNSDFIIK |
| | IPFGRWQVKKEIDKYRPWEKFDFEQVQKSPKPISLLLSTQRRKRNKGWSKDEGTEAEIK |
| | KVMNGDYQTSYIEVKRGSKICEKSAWMLNLSIDVPKIDKGVDPSIIGGIAVGVRSPLVCA |
| | INNAFSRYSISDNDLFHFNKKMFARRRILLKKNRHKRAGHGAKNKLKPITILTEKSERFR |
| | KKLIERWACEIADFFIKNKVGTVQMENLESMKRKEDSYFNIRLRGFWPYAEMQNKIEFK |
| | LKQYGIEIRKVAPNNTSKTCSKCGHLNNYFNFEYRKKNKFPHFKCEKCNFKENAAYNA |
| | ALNISNPKLKSTKERP |
| 23 | MIKVYRYEIVKPLDLDWQEISEIFRQLQKQAAEIYNQSLIELYYEIFIKGKGIANASSVEHY |
| | LSRVCYRRAAELFKNAAIASGLRSKIKSNFRLKELKNMKSGLPTTKSDNFPIPLVKQKGG |
| | QYTGFEISNHNSDFIIKIPFGRWQVKKEIDKYRPWEKFDFEQVQKSPKPISLLLSTQRRKR |
| | NKGWSKDEGTEAEIKKVMNGDYQTSYIEVKRGSKICEKSAWMLNLSIDVPKIDKGVDPS |
| | IIGGIAVGVRSPLVCAINNAFSRYSISDNDLFHFNKKMFARRRILLKKNRHKRAGHGAKN |
| | KLKPITILTEKSERFRKKLIERWACEIADFFIKNKVGTVQMENLESMKRKEDSYFNIRLRG |
| | FWPYAEMQNKIEFKLKQYGIEIRKVAPNNTSKTCSKCGHLNNYFNFEYRKKNKFPHFKC |
| | EKCNFKENAAYNAALNISNPKLKSTKERP |
| 24 | MITVRKIKLTIMGDKDTRNSQYKWIRDEQYNQYRALNMGMTYLAVNDAVFWQEISEIF |
| | RQLQKQAAEIYNQSLIELYYEIFIKGKGIANASSVEHYLSRVCYRRAAELFKNAAIASGLR |
| | SKIKSNFRLKELKNMKSGLPTTKSDNFPIPLVKQKGGQYTGFEISNHNSDFIIKIPFGRWQ |
| | VKKEIDKYRPWEKFDFEQVQKSPKPISLLLSTQRRKRNKGWSKDEGTEAEIKKVMNGD |
| | YQTSYIEVKRGSKICEKSAWMLNLSIDVPKIDKGVDPSIIGGIAVGVRSPLVCAINNAFSR |
| | YSISDNDLFHFNKKMFARRRILLKKNRHKRAGHGAKNKLKPITILTEKSERFRKKLIERW |
| | ACEIADFFIKNKVGTVQMENLESMKRKEDSYFNIRLRGFWPYAEMQNKIEFKLKQYGIEI |
| | RKVAPNNTSKTCSKCGHLNNYFNFEYRKKNKFPHFKCEKCNFKENAAYNAALNISNPK |
| | LKSTKERP |
| 25 | MGESVKAIKLKILDMFLDPECTKQDDNWQEISEIFRQLQKQAAEIYNQSLIELYYEIFIKG |
| | KGIANASSVEHYLSRVCYRRAAELFKNAAIASGLRSKIKSNFRLKELKNMKSGLPTTKSD |
| | NFPIPLVKQKGGQYTGFEISNHNSDFIIKIPFGRWQVKKEIDKYRPWEKFDFEQVQKSPKP |
| | ISLLLSTQRRKRNKGWSKDEGTEAEIKKVMNGDYQTSYIEVKRGSKICEKSAWMLNLSI |
| | DVPKIDKGVDPSIIGGIAVGVRSPLVCAINNAFSRYSISDNDLFHFNKKMFARRRILLKKN |
| | RHKRAGHGAKNKLKPITILTEKSERFRKKLIERWACEIADFFIKNKVGTVQMENLESMK |
| | RKEDSYFNIRLRGFWPYAEMQNKIEFKLKQYGIEIRKVAPNNTSKTCSKCGHLNNYFNFE |
| | YRKKNKFPHFKCEKCNFKENAAYNAALNISNPKLKSTKERP |
| 26 | MKYTKVMRYQIIKPLNAEWQEISEIFRQLQKQAAEIYNQSLIELYYEIFIKGKGIANASSV |
| | EHYLSRVCYRRAAELFKNAAIASGLRSKIKSNFRLKELKNMKSGLPTTKSDNFPIPLVKQ |
| | KGGQYTGFEISNHNSDFIIKIPFGRWQVKKEIDKYRPWEKFDFEQVQKSPKPISLLLSTQR |
| | RKRNKGWSKDEGTEAEIKKVMNGDYQTSYIEVKRGSKICEKSAWMLNLSIDVPKIDKG |
| | VDPSIIGGIAVGVRSPLVCAINNAFSRYSISDNDLFHFNKKMFARRRILLKKNRHKRAGH |
| | GAKNKLKPITILTEKSERFRKKLIERWACEIADFFIKNKVGTVQMENLESMKRKEDSYFN |
| | IRLRGFWPYAEMQNKIEFKLKQYGIEIRKVAPNNTSKTCSKCGHLNNYFNFEYRKKNKF |
| | PHFKCEKCNFKENAAYNAALNISNPKLKSTKERP |
| 27 | MTLLVKVVKIHLISEQFDKAGNRIDYEEISEIFRQLQKQAAEIYNQSLIELYYEIFIKGKGIA |
| | NASSVEHYLSRVCYRRAAELFKNAAIASGLRSKIKSNFRLKELKNMKSGLPTTKSDNFPI |
| | PLVKQKGGQYTGFEISNHNSDFIIKIPFGRWQVKKEIDKYRPWEKFDFEQVQKSPKPISLL |
| | LSTQRRKRNKGWSKDEGTEAEIKKVMNGDYQTSYIEVKRGSKICEKSAWMLNLSIDVP |
| | KIDKGVDPSIIGGIAVGVRSPLVCAINNAFSRYSISDNDLFHFNKKMFARRRILLKKNRHK |
| | RAGHGAKNKLKPITILTEKSERFRKKLIERWACEIADFFIKNKVGTVQMENLESMKRKED |
| | SYFNIRLRGFWPYAEMQNKIEFKLKQYGIEIRKVAPNNTSKTCSKCGHLNNYFNFEYRK |
| | KNKFPHFKCEKCNFKENAAYNAALNISNPKLKSTKERP |
| 28 | MAKNTITKTLKLRIVRPYYSQEIEKIVAEEKNRREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 29 | MAKNTITKTLKLRIVRPYYSAEVEKIVAEEKNNREKIALDKNKDKVKEACSKHLKVAAY |
| | CTTQVERNACLFCKARKLDDKFYKKLRGQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 30 | MAKNTITKTLKLRIVRPYYSAEIEKIVADEKNRREKIALEKNKDKVKEACSKHLKVAAY |
| | CTTQVERNACLFCKARKLDDKFYQKLRKQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 31 | MAKNTITKTLKLRIVRPYNSQEVEKIVAEEKNRREKIALDKNKDKVKEACSKHLKVAAY |
| | CTTQVERNACLFCKARKLDDKFYQKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 32 | MAKNTITKTLKLRIVRPYNSQEVEKIVAEEKNNREKIALDKNKDKVKEACSKHLKVAAY |
| | CTTQVERNACLFCKARKLDDKFYKKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 33 | MAKNTITKTLKLRIVRPYNSQEVEKIVAEEKNNREKIALDKNKDKVKEACSKHLKVAAY |
| | CTTQVERNACLFCKARKLDDKFYKKLRGQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 34 | MAKNTITKTLKLRIVRPYYSAEVEKIVAEEKNNREKIALDKNKDKVKEACSKHLKVAAY |
| | CTTQVERNACLFCKARKLDDKFYKKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 35 | MAKNTITKTLKLRIVRPYNSAEIEKIVADEKNRREKIALDKNKDKVKEACSKHLKVAAY |
| | CTTQVERNACLFCKARKLDDKFYKKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 36 | MAKNTITKTLKLRIVRPYNSAEIEKIVADEKNRREKIALDKNKDKVKEACSKHLKVAAY |
| | CTTQVERNACLFCKARKLDDKFYKKLRGQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 37 | MAKNTITKTLKLRIVRPYNSQEIEKIVAEEKNRREKIALDKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 38 | MAKNTITKTLKLRIVRPYNSAEIEKIVAEEKNRREKIALDKNKDKVKEACSKHLKVAAY |
| | CTTQVERNACLFCKARKLDDKFYKKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 39 | MAKNTITKTLKLRIVRPYNSAEVEKIVAEEKNRREKIALDKNKDKVKEACSKHLKVAAY |
| | CTTQVERNACLFCKARKLDDKFYKKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 40 | MAKNTITKTLKLRIVRPYNSAEVEKIVADEKNRREKIALDKNKDKVKEACSKHLKVAAY |
| | CTTQVERNACLFCKARKLDDKFYKKLRKQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 41 | MAKNTITKTLKLRIVRPYYSAEIEKIVADEKNRREKIALDKNKDKVKEACSKHLKVAAY |
| | CTTQVERNACLFCKARKLDDKFYQKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 42 | MAKNTITKTLKLRIVRPYYSAEIEKIVAEEKNRREKIALEKNKDKVKEACSKHLKVAAY |
| | CTTQVERNACLFCKARKLDDKFYQKLRKQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 43 | MAKNTITKTLKLRIVRPYYSAEIEKIVAEEKNNREKIALDKNKDKVKEACSKHLKVAAY |
| | CTTQVERNACLFCKARKLDDKFYQKLRGQFPDAVFWQEISEIFRQLQKQAREIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 44 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAARLFKNAAIASGLRSKIKSNFRLKELKNMK |
| | SGLPTTKSDNFPIPLVKQKGGQYTGFKISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 45 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFKISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 46 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAGLFKNAAIASGLRSKIKSNFRLKELKNMK |
| | SGLPTTKSDNFPIPLVKQKGGQYTGFKISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 47 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAARLFKNAAIASGLRSKIKSNFRLKELKNMK |
| | SGLPTTKSDNFPIPLVKQKGGQYTGFRISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 48 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFRISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 49 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAGLFKNAAIASGLRSKIKSNFRLKELKNMK |
| | SGLPTTKSDNFPIPLVKQKGGQYTGFRISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 50 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAARLFKNAAIASGLRSKIKSNFRLKELKNMK |
| | SGLPTTKSDNFPIPLVKQKGGQYTGFSISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 51 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFSISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 52 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAGLFKNAAIASGLRSKIKSNFRLKELKNMK |
| | SGLPTTKSDNFPIPLVKQKGGQYTGFSISNHNSDFIIKIPFGRWQVKKEIDKYRPWEKFDF |
| | RQVQKSPKPISLLLSTQRRKRNKGWSKDEGTEAEIRKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 53 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLENFNKKMFA |
| | RRRILLKKNRHKRGGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 54 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIEGGDLFHFNKKMFA |
| | RRRILLKKNRHKRAGHGAKNKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 55 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIEGGDLENFNKKMFA |
| | RRRILLKKNRHKRGGHGRDKKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 56 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIEGGDLENFNKKMFA |
| | RRRILLKKNRHKRAGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 57 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGRDKKLKPIEQLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 58 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLENFNKKMFA |
| | RRRILLKKNRHKRAGHGRDKKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 59 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLEHFNKKMFA |
| | RRRILLKKNRHKRKGHGAKNKLKPIETLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 60 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDGGDLEHFNKKMFA |
| | RRRILLKKNRHKRKGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 61 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDGGDLFHFNKKMFA |
| | RRRILLKKNRHKRAGHGAKNKLKPIETLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 62 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDGGDLEHFNKKMFA |
| | RRRILLKKNRHKRKGHGAKNKLKPIETLTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 63 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSIDSNDLFKFNKKMFAR |
| | RRILLKKNRHKRKGHGAKNKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 64 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSIDSNDLFHFNKKMFAR |
| | RRILLKKNRHKRAGHGAAHKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 65 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSIDSNDLFKFNKKMFAR |
| | RRILLKKNRHKRAGHGAAHKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 66 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDSNDLFKFNKKMFAR |
| | RRILLKKNRHKRAGHGAAHKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 67 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSIDSNDLFKFNKKMFAR |
| | RRILLKKNRHKRAGHGAKNKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 68 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSISDNDLFKFNKKMFAR |
| | RRILLKKNRHKRKGHGAKNKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 69 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIDSNDLFKFNKKMFAR |
| | RRILLKKNRHKRKGHGAKNKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 70 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFKFNKKMFAR |
| | RRILLKKNRHKRKGHGAAHKLKPITELTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNISNPKLKSTKERP |
| 71 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLERFNKKMFA |
| | RRRILLKKNRHKRKGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 72 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLERFNKKMFA |
| | RRRILLKKNRHKRAGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 73 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLEKFNKKMFA |
| | RRRILLKKNRHKRAGHGAKNKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 74 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLFHFNKKMFA |
| | RRRILLKKNRHKRAGHGRKKKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 75 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLEKFNKKMFA |
| | RRRILLKKNRHKRAGHGRKKKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 76 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSIKGGDLEKFNKKMFA |
| | RRRILLKKNRHKRAGHGRKKKLKPITILTEKSERFRKKLIERWACEIADFFIKNKVGTVQ |
| | MENLESMKRKEDSYFNIRLRGFWPYAEMQNKIEFKLKQYGIEIRKVAPNNTSKTCSKCG |
| | HLNNYFNFEYRKKNKFPHFKCEKCNFKENAAYNAALNISNPKLKSTKERP |
| 77 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWAKEIADFFIKNKVGTVQM |
| | EDLSTMKRKEDSYFNIRLRGFWPYYEMQNKIEFKLKQYGIEIRKVAPNNTSQLCSKCGH |
| | LNNYFNFEYRKKNKFPKFKCEKCNFKENAAYNAARNISTPDIKSTKERP |
| 78 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLESMKRKEDSYFNIRLRGFWPYAEMQNKIEFKLKQYGIEIRKVAPNNTSQLCSKCGH |
| | LNNYFNFEYRKKNKFPHFKCEKCNFKENAAYNAALNISNPDIKSTKERP |
| 79 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWAKEIADFFIKNKVGTVQM |
| | EDLSTMKRKEDSYFNIRLRGFWPYYEMQNKIEFKLKQYGIEIRKVAPNNTSKTCSKCGH |
| | LNNYFNFEYRKKNKFPHFKCEKCNFKENAAYNAALNISNPKLKSTKERP |
| 80 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSQLCSKCGH |
| | LNNYFNFEYRKKNKFPKFKCEKCNFKENAAYNAALNISNPDIKSTKERP |
| 81 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSQLCSKCGH |
| | LNNYFNFEYRKKNKFPHFKCEKCNFKENAAYNAALNISTPDIKSTKERP |
| 82 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLSTMKRKEDSYFNIRLRGFWPYYEMQNKIEFKLKQYGIEIRKVAPNNTSKTCSKCGH |
| | LNNYFNFEYRKKNKFPKFKCEKCNFKENAAYNAALNISNPKLKSTKERP |
| 83 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLESMKRKEDSYFNIRLRGFWPYAEMQNKIEFKLKQYGIEIRKVAPNNTSQLCSKCGH |
| | LNNYFNFEYRKKNKFPKFKCEKCNFKENAAYNAALNISTPDIKSTKERP |
| 84 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPKFKCEKCNFKENAAYNAARNISTPDIKSTKERP |
| 85 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWSRYIADFFIKNKVGTVQM |
| | EDLESMKRKEDSYFNIRLRGFWPYYEMQNKIEFKLKQYGIKIRKVAPNNTSQRCSKCGH |
| | LNNYFNFEYRKKNKFPHFKCEKCNFKANAAYNAARNISNPNIKSTKERP |
| 86 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACYIADFFIKNKVGTVQM |
| | EDLESMKRKEDSYFNIRLRGFWPYAEMQNKIEFKLKQYGIEIRKVAPNNTSQRCSKCGH |
| | LNNYFNFEYRKKNKFPHFKCEKCNFKENAAYNAARNISNPNIKSTKERP |
| 87 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACYIADFFIKNKVGTVQM |
| | ENLESMKRKEDSYFNIRLRGFWPYAEMQNKIEFKLKQYGIEIRKVAPNNTSQRCSKCGH |
| | LNNYFNFEYRKKNKFPHFKCEKCNFKRNAAYNAARNISNPKLKSTKERP |
| 88 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACYIADFFIKNKVGTVQM |
| | EDLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAARNISNPNIKSTKERP |
| 89 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWARYIADFFIKNKVGTVQM |
| | EDLESMKRKEDSYFNIRLRGFWPYYEMQNKIEFKLKQYGIKIRKVAPNNTSKTCSKCGH |
| | LNNYFNFEYRKKNKFPHFKCEKCNFKENAAYNAALNISNPKLKSTKERP |
| 90 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSQRCSKCGH |
| | LNNYFNFEYRKKNKFPHFKCEKCNFKRNAAYNAARNISNPNIKSTKERP |
| 91 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLESMKRKEDSYFNIRLRGFWPYAEMQNKIEFKLKQYGIEIRKVAPNNTSQRCSKCGH |
| | LNNYFNFEYRKKNKFPHFKCEKCNFKENAAYNAARNISNPNIKSTKERP |
| 92 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKRNAAYNAARNISNPNIKSTKERP |
| 93 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWANRIADFFIKNKVGTVQM |
| | ENLESMKRKEDSYFNIRLRGFWPYAEMQNKIEFKLKQYGIKIRKVAPNNTSQRCSKCGH |
| | LNNYFNFEYRKKNKFPHFKCEKCNFKRNAAYNAAKNISNPKLKSTKERP |
| 94 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSQRCSKCGH |
| | LNNYFNFEYRKKNKFPHFKCEKCNFKRNAAYNAAKNISNPKLKSTKERP |
| 95 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIKIRKVAPNNTSQRCSKCGH |
| | LNNYFNFEYRKKNKFPHFKCEKCNFKRNAAYNAAKNISNPKLKSTKERP |
| 96 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWANRIADFFIKNKVGTVQM |
| | ENLESMKRKEDSYFNIRLRGFWPYAEMQNKIEFKLKQYGIKIRKVAPNNTSQRCSKCGH |
| | LNNYFNFEYRKKNKFPHFKCEKCNFKENAAYNAALNISNPKLKSTKERP |
| 97 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWANRIADFFIKNKVGTVQM |
| | ENLESMKRKEDSYFNIRLRGFWPYAEMQNKIEFKLKQYGIEIRKVAPNNTSQRCSKCGH |
| | LNNYFNFEYRKKNKFPHFKCEKCNFKRNAAYNAALNISNPKLKSTKERP |
| 98 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWSRFIADFFIKNKVGTVQM |
| | EDLESMKRKEDSYFNIRLRGFWPYYEMQNKIEFKLKQYGIEIRKVAPNNTSQRCSKCGH |
| | LNNYFNFEYRKKNKFPHFKCEKCNFKENAAYNAARNISNPNIKSTKERP |
| 99 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIDVQLYSKEYSKDSGKGTHRYFLLS |
| | TQRRKRNKGWSKDEGTEAEIKKVMNGDYQTSYIEVKRGSKICEKSAWMLNLSIDVPKID |
| | KGVDPSIIGGIAVGVRSPLVCAINNAFSRYSISDNDLFHFNKKMFARRRILLKKNRHKRA |
| | GHGAKNKLKPITILTEKSERFRKKLIERWACEIADFFIKNKVGTVQMENLESMKRKEDSY |
| | FNIRLRGFWPYAEMQNKIEFKLKQYGIEIRKVAPNNTSKTCSKCGHLNNYFNFEYRKKN |
| | KFPHFKCEKCNFKENAAYNAALNISNPKLKSTKERP |
| 100 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIASLSLLSNPAKQEMNVKRKISLLLS |
| | TQRRKRNKGWSKDEGTEAEIKKVMNGDYQTSYIEVKRGSKICEKSAWMLNLSIDVPKID |
| | KGVDPSIIGGIAVGVRSPLVCAINNAFSRYSISDNDLFHFNKKMFARRRILLKKNRHKRA |
| | GHGAKNKLKPITILTEKSERFRKKLIERWACEIADFFIKNKVGTVQMENLESMKRKEDSY |
| | FNIRLRGFWPYAEMQNKIEFKLKQYGIEIRKVAPNNTSKTCSKCGHLNNYFNFEYRKKN |
| | KFPHFKCEKCNFKENAAYNAALNISNPKLKSTKERP |
| 101 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPIYERKPNRSIVGGLAVGIRSPLVCAINNSFSRYSVDSNDVFKFSKQVFAF |
| | RRRLLSKNSLKRKGHGAAHKLEPITEMTEKNDKFRKKIIERWAKEVTNFFVKNQVGIVQ |
| | IEDLSTMKDREDHFFNQYLRGFWPYYQMQTLIENKLKEYGIEVKRVQAKYTSQLCSNPN |
| | CRYWNNYFNFEYRKVNKFPKFKCEKCNLEISAAYNAARNLSTPDIEKFVAKATKGINLP |
| | EK |
| 102 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPTHETVLDPDKVMGVALGVAKAVYWAFNSSYKRGCIDGGEIEHFRKMI |
| | RARRVSIQNQIKHSGDARKGHGRKRALKPIETLSEKEKNFRDTINHRYANRIVEAAIKQG |
| | CGTIQIENLEGIADTTGSKFLKNWPYYDLQTKIVNKAKEHGITVVAINPQYTSQRCSMCG |
| | YIEKTNRSSQAVFECKQCGYGSRTICINCRHVQVSGDVCEECGGIVKKENVNAAYNAAK |
| | NISTPYIDQIIMEKCLELGIPYRSITCKECGHIQASGNTCEVCGSTNILKPKK |
| 103 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPQTRVLDLNKIMGIALGVAVAVYMAFQHTPARYKLEGGEIENFRRQVE |
| | SRRISMLRQGKYAGGARGGHGRDKRIKPIEQLRDKIANFRDTTNHRYSRYIVDMAIKEG |
| | CGTIQMEDLTNIRDIGSRFLQNWTYYDLQQKIIYKAEEAGIKVIKIDPQYTSQRCSECGNI |
| | DSGNRIGQAIFKCRACGYEANAAYNAARNIAIPNIDKIIAESIK |
| 104 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPIDIVNKKVSGRVVGVALGLKIPAYCALNDVEYIKKSIGRIDDFLKVRTQ |
| | MQSRRRRLQIAIQSAKGGKGRVNKLQALERFAEKEKNFAKTYNHFLSSNIVKFAVSNQA |
| | EQINMELLSLKETQNKSILRNWSYYQLQTMIEYKAQREGIKVKYIDPYHTSQTCSKCGN |
| | YEEGQRESQADFICKKCGYKVNAAYNAARNIAMSNKYITKKEESKYYKIKESMV |
| 105 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVETKETALDPNNVMGVALGIVYPVYIAFNNSLHRYHIKGGEIERFRRQVE |
| | KRKRELLNQGKYCGDGRKGHGYATRTKSIESISDKIARFRDTCNHKYSRFIVDMALKHN |
| | CGIIQMEDLTGISKESTFLKNWTYYDLQQKIEYKAREAGIQVIKIEPQYTSQRCSKCGYID |
| | KENRQEQATFKCIECGFKTNAAYNAARNIAIPNIDKIIRKTLKMQ |
| 106 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVNRSIVGGLAVGIRSPLVCAINNSFSRYSVDSNDVFKFSKQVFA |
| | FRRRLLSKNSLKRKGHGAAHKLEPITEMTEKNDKFRKKIIERWAKEVTNFFVKNQVGIV |
| | QIEDLSTMKDREDHFFNQYLRGFWPYYQMQTLIENKLKEYGIEVKRVQAKYTSQLCSNP |
| | NCRYWNNYFNFEYRKVNKFPKFKCEKCNLEISAAYNAARNLSTPDIEKFVAKATKGINL |
| | PEK |
| 107 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPDKVMGVALGVAKAVYWAFNSSYKRGCIDGGEIEHFRKMI |
| | RARRVSIQNQIKHSGDARKGHGRKRALKPIETLSEKEKNFRDTINHRYANRIVEAAIKQG |
| | CGTIQIENLEGIADTTGSKFLKNWPYYDLQTKIVNKAKEHGITVVAINPQYTSQRCSMCG |
| | YIEKTNRSSQAVFECKQCGYGSRTICINCRHVQVSGDVCEECGGIVKKENVNAAYNAAK |
| | NISTPYIDQIIMEKCLELGIPYRSITCKECGHIQASGNTCEVCGSTNILKPKK |
| 108 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDLNKIMGIALGVAVAVYMAFQHTPARYKLEGGEIENFRRQV |
| | ESRRISMLRQGKYAGGARGGHGRDKRIKPIEQLRDKIANFRDTTNHRYSRYIVDMAIKE |
| | GCGTIQMEDLTNIRDIGSRFLQNWTYYDLQQKIIYKAEEAGIKVIKIDPQYTSQRCSECGN |
| | IDSGNRIGQAIFKCRACGYEANAAYNAARNIAIPNIDKIIAESIK |
| 109 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPNNVMGVALGIVYPVYIAFNNSLHRYHIKGGEIERFRRQVE |
| | KRKRELLNQGKYCGDGRKGHGYATRTKSIESISDKIARFRDTCNHKYSRFIVDMALKHN |
| | CGIIQMEDLTGISKESTFLKNWTYYDLQQKIEYKAREAGIQVIKIEPQYTSQRCSKCGYID |
| | KENRQEQATFKCIECGFKTNAAYNAARNIAIPNIDKIIRKTLKMQ |
| 110 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDKEKILGVAVGVNCPLVASVFGDRDRFIIKGGEIEKFRKSVEA |
| | RRRSMLEQTKYCGDGRIGHGRKKRTEPALNIGDKIARFRDTTNHKYSRALIEYAVKKGC |
| | GTIQMEKLTGITSKSDRFLKDWTYYDLQTKIENKAKEVGINVVYIAPKYTSQRCSKCGYI |
| | HKDNRPNQAKFRCLECDFESNAAYNASQNIGIKNIDKIIEKDLQKQESEVQVNENK |
| 111 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAELFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | ENLESMKRKEDSYFNIRLRGFWPYAEMQNKIEFKLKQYGIEIRKVAPNNTSKTCSKCGH |
| | LNNYFNFEYRKKNKFPHFKCEKCNFKENAAYNAALNI |
| 112 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 113 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 114 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 115 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 116 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 117 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 118 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 119 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 120 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 121 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 122 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 123 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 124 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 125 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 126 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 127 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 128 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 129 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 130 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 131 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 132 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 133 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 134 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 135 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 136 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 137 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 138 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 139 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 140 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 141 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 142 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 143 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 144 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 145 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 146 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 147 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 148 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 149 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 150 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 151 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 152 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 153 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 154 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 155 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 156 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 157 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 158 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 159 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 160 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 161 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 162 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 163 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 164 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 165 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 166 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 167 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 168 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 169 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 170 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 171 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 172 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 173 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 174 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 175 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 176 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 177 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 178 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 179 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 180 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 181 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNSFSRYSISDNDLFKFNKKMFARRRILLKKNRHKRKGHGAKNKLKPITELTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 182 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 183 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 184 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISTPDIKSTKERP |
| 185 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNISNPNIKSTKERP |
| 186 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 187 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNI |
| 188 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 189 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNI |
| 190 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQRCSKCGHLNNYFNFEYRKKNKFPHFKCEKCNFKENAAYN |
| | AARNI |
| 191 | MAKNTITKTLKLRIVRPYNSQEIEKIVAEEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNI |
| 192 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNSFSRYSISDNDLFKFNKKMFAR |
| | RRILLKKNRHKRKGHGAKNKLKPITELTEKSERFRKKLIERWACEIADFFIKNKVGTVQM |
| | EDLESMKRKEDSYFNIRLRGFWPYAEMQNKIEFKLKQYGIEIRKVAPNNTSQLCSKCGH |
| | LNNYFNFEYRKKNKFPHFKCEKCNFKENAAYNAALNISNPDIKSTKERP |
| 193 | MAKNTITKTLKLRIVRPYNSAEVEKIVADEKNNREKIALEKNKDKVKEACSKHLKVAAY |
| | CTTQVERNACLFCKARKLDDKFYQKLRGQFPDAVFWQEISEIFRQLQKQAAEIYNQSLIE |
| | LYYEIFIKGKGIANASSVEHYLSRVCYRRAAALFKNAAIASGLRSKIKSNFRLKELKNMK |
| | SGLPTTKSDNFPIPLVKQKGGQYTGFEISNHNSDFIIKIPFGRWQVKKEIDKYRPWEKFDF |
| | EQVQKSPKPISLLLSTQRRKRNKGWSKDEGTEAEIKKVMNGDYQTSYIEVKRGSKICEKS |
| | AWMLNLSIDVPKIDKGVDPSIIGGIAVGVRSPLVCAINNAFSRYSISDNDLFHFNKKMFAR |
| | RRILLKKNRHKRAGHGAKNKLKPITILTEKSERFRKKLIERWACEIADFFIKNKVGTVQM |
| | EDLESMKRKEDSYFNIRLRGFWPYAEMQNKIEFKLKQYGIEIRKVAPNNTSQLCSKCGH |
| | LNNYFNFEYRKKNKFPHFKCEKCNFKENAAYNAALNISNPDIKSTKERP |
| 194 | MAKNTITKTLKLRIVRPYNSAEIEKIVADEKERRKQAGGTGELDDKFYKKLRKQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 195 | MAKNTITKTLKLRIVRPYNSAEIEKIVADEKERRKQAGGTGELDDKFYKKLRKQFPDAV |
| | FWQEISEIFRQLQKQAAEIYNQSLIELYYEIFIKGKGIANASSVEHYLSRVCYRRAAALFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 196 | MAKNTITKTLKLRIVRPYNSAEIEKIVADEKERRKQAGGTGELDDKFYKKLRKQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |
| 197 | MAKNTITKTLKLRIVRPYNSAEIEKIVADEKERRKQAGGTGELDDKFYKKLRKQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPKFKCEKCNFKENAAYN |
| | AALNISNPDIKSTKERP |
| 198 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMEDLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPKLKSTKERP |
| 199 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPKLKSTKERP |
| 200 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSKTCSKCGHLNNYFNFEYRKKNKFPHFKCEKCNFKENAAYN |
| | AALNISNPDIKSTKERP |
| 201 | MAKNTITKTLKLRIVRPYNSAEVEKIVADEKERRKQAGGTGELDDKFYQKLRGQFPDAV |
| | FWQEISEIFRQLQKQAAEIYNQSLIELYYEIFIKGKGIANASSVEHYLSRVCYRRAAELFK |
| | NAAIASGLRSKIKSNFRLKELKNMKSGLPTTKSDNFPIPLVKQKGGQYTGFEISNHNSDFII |
| | KIPFGRWQVKKEIDKYRPWEKFDFEQVQKSPKPISLLLSTQRRKRNKGWSKDEGTEAEI |
| | KKVMNGDYQTSYIEVKRGSKICEKSAWMLNLSIDVPKIDKGVDPSIIGGIAVGVRSPLVC |
| | AINNAFSRYSISDNDLFHFNKKMFARRRILLKKNRHKRAGHGAKNKLKPITILTEKSERF |
| | RKKLIERWACEIADFFIKNKVGTVQMENLESMKRKEDSYFNIRLRGFWPYAEMQNKIEF |
| | KLKQYGIEIRKVAPNNTSQLCSKCGHLNNYFNFEYRKKNKFPHFKCEKCNFKENAAYN |
| | AALNISNPDIKSTKERP |

TABLE 2
Non-limiting examples of a guide nucleic acid scaffold sequence
or a derivative thereof.

| SEQ | |
|---|---|
| ID NO | Guide nucleic acid (NA) scaffold sequence (without spacer) |
| 500 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATTCATTTGAATGAAGGAATGCAAC |
| 501 | GAACCGCTTCACCAAAAGCTGTCCCTTAGGGGATTAGAACTTGAGTGAAGGTGGGCT |
| | GCTTGCATCAGCCTAATGTCGAGAAGTGCTTTCTTCGGAAAGTAACCCTCGAAACAA |
| | ATTCATTTGAATGAAGGAATGCAAC |
| 502 | AACCGCTTCACCAAAAGCTGTCCCTTAGGGGATTAGAACTTGAGTGAAGGTGGGCTG |
| | CTTGCATCAGCCTAATGTCGAGAAGTGCTTTCTTCGGAAAGTAACCCTCGAAACAAA |
| | TTCATTTGAATGAAGGAATGCAAC |
| 503 | ACCGCTTCACCAAAAGCTGTCCCTTAGGGGATTAGAACTTGAGTGAAGGTGGGCTGC |
| | TTGCATCAGCCTAATGTCGAGAAGTGCTTTCTTCGGAAAGTAACCCTCGAAACAAAT |
| | TCATTTGAATGAAGGAATGCAAC |
| 504 | CCGCTTCACCAAAAGCTGTCCCTTAGGGGATTAGAACTTGAGTGAAGGTGGGCTGCT |
| | TGCATCAGCCTAATGTCGAGAAGTGCTTTCTTCGGAAAGTAACCCTCGAAACAAATT |
| | CATTTGAATGAAGGAATGCAAC |
| 505 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAGCAATAAGGAATGCAAC |
| 506 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAGAAAGGAATGCAAC |
| 507 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATCTTCGGATTAAGGAATGCAAC |
| 508 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATTGCAAAAGGAATGCAAC |
| 509 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATTCGTTAAGGAATGCAAC |
| 510 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATGCAAAGGAATGCAAC |
| 511 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAAGCAATTAAGGAATGCAAC |
| 512 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAAGAAAGGAATGCAAC |
| 513 | GGCTTCACTGATAAAGTGGAGAACCGCTTCACTTAGAGTGAAGGTGGGCTGCTTGCA |
| | TCAGCCTAATGTCGAGAAGTGCTTTCTTCGGAAAGTAACCCTCGAAACAAATTCATT |
| | TGAATGAAGGAATGCAAC |
| 514 | GGCTTCACTGATAAAGTGGAGAACCGCTTCACTTCGAGTGAAGGTGGGCTGCTTGCA |
| | TCAGCCTAATGTCGAGAAGTGCTTTCTTCGGAAAGTAACCCTCGAAACAAATTCATT |
| | TGAATGAAGGAATGCAAC |
| 515 | GGCTTCACTGATAAAGTGGAGAACCGCTTCACTTCGGTGAAGGTGGGCTGCTTGCAT |
| | CAGCCTAATGTCGAGAAGTGCTTTCTTCGGAAAGTAACCCTCGAAACAAATTCATTT |
| | GAATGAAGGAATGCAAC |
| 516 | GGCTTCACTGATAAAGTGGAGAACCGCTTCACCTTAGGAGTGAAGGTGGGCTGCTTG |
| | CATCAGCCTAATGTCGAGAAGTGCTTTCTTCGGAAAGTAACCCTCGAAACAAATTCA |
| | TTTGAATGAAGGAATGCAAC |
| 517 | GGCTTCACTGATAAAGTGGAGAACCGCTTCACCTTCGGAGTGAAGGTGGGCTGCTTG |
| | CATCAGCCTAATGTCGAGAAGTGCTTTCTTCGGAAAGTAACCCTCGAAACAAATTCA |
| | TTTGAATGAAGGAATGCAAC |
| 518 | GGCTTCACTGATAAAGTGGAGAACCGCTTCACGCTTCGGCAGTGAAGGTGGGCTGCT |
| | TGCATCAGCCTAATGTCGAGAAGTGCTTTCTTCGGAAAGTAACCCTCGAAACAAATT |
| | CATTTGAATGAAGGAATGCAAC |
| 519 | ACCGCTTCACCAAAAGCTGTCCTTAGGGATTAGAACTTGAGTGAAGGTGGGCTGCTT |
| | GCATCAGCCTAATGTCGAGAAGTGCTTTCTTCGGAAAGTAACCCTCGAAACAAATTC |
| | ATTTGAATGAAGGAATGCAAC |
| 520 | ACCGCTTCACCAAAAGCTGTCTTAGGATTAGAACTTGAGTGAAGGTGGGCTGCTTGC |
| | ATCAGCCTAATGTCGAGAAGTGCTTTCTTCGGAAAGTAACCCTCGAAACAAATTCAT |
| | TTGAATGAAGGAATGCAAC |
| 521 | ACCGCTTCACCAAAAGCTGTTTAGATTAGAACTTGAGTGAAGGTGGGCTGCTTGCAT |
| | CAGCCTAATGTCGAGAAGTGCTTTCTTCGGAAAGTAACCCTCGAAACAAATTCATTT |
| | GAATGAAGGAATGCAAC |
| 522 | ACCGCTTCACCAAAAGCTGTTAGTTAGAACTTGAGTGAAGGTGGGCTGCTTGCATCA |
| | GCCTAATGTCGAGAAGTGCTTTCTTCGGAAAGTAACCCTCGAAACAAATTCATTTGA |
| | ATGAAGGAATGCAAC |
| 523 | ACCGCTTCACCAAAAGCTTTAGAGAACTTGAGTGAAGGTGGGCTGCTTGCATCAGCC |
| | TAATGTCGAGAAGTGCTTTCTTCGGAAAGTAACCCTCGAAACAAATTCATTTGAATG |
| | AAGGAATGCAAC |
| 524 | ACCGCTTCACCAAAAGCTTCGGCACTTGAGTGAAGGTGGGCTGCTTGCATCAGCCTA |
| | ATGTCGAGAAGTGCTTTCTTCGGAAAGTAACCCTCGAAACAAATTCATTTGAATGAA |
| | GGAATGCAAC |
| 525 | ACCGCTTCACCAAAAGTTCGCACTTGAGTGAAGGTGGGCTGCTTGCATCAGCCTAAT |
| | GTCGAGAAGTGCTTTCTTCGGAAAGTAACCCTCGAAACAAATTCATTTGAATGAAGG |
| | AATGCAAC |
| 526 | ACCGCTTCACCAAAATTCGTCTTGAGTGAAGGTGGGCTGCTTGCATCAGCCTAATGT |
| | CGAGAAGTGCTTTCTTCGGAAAGTAACCCTCGAAACAAATTCATTTGAATGAAGGAA |
| | TGCAAC |
| 527 | ACCGCTTCACCAAGTTCGCTTGAGTGAAGGTGGGCTGCTTGCATCAGCCTAATGTCG |
| | AGAAGTGCTTTCTTCGGAAAGTAACCCTCGAAACAAATTCATTTGAATGAAGGAATG |
| | CAAC |
| 528 | ACCGCTTCACCAATTCGTTGAGTGAAGGTGGGCTGCTTGCATCAGCCTAATGTCGAG |
| | AAGTGCTTTCTTCGGAAAGTAACCCTCGAAACAAATTCATTTGAATGAAGGAATGCA |
| | AC |
| 529 | ACCGCTTCACCATTCGTGAGTGAAGGTGGGCTGCTTGCATCAGCCTAATGTCGAGAA |
| | GTGCTTTCTTCGGAAAGTAACCCTCGAAACAAATTCATTTGAATGAAGGAATGCAAC |
| 530 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATTCATTTGAATGAAGGAATGCAAC |
| — | TT |
| 532 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATTCATTTGAATGAAGGAATGCAAC |
| — | TTTTA |
| 534 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATTCATTTGAATGAAGGAATGCAAC |
| — | TTTTG |
| 536 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAATTCATTTGAATGAAGGAATGCAAC |
| 537 | TTTTATTTTT |
| 538 | GGCTTCACTGATAAAGTGGAGAACCGCTTCACCGAGTGAAGGTGGGCTGCTTGCATC |
| | AGCCTAATGTCGAGAAGTGCTTTCTTCGGAAAGTAACCCTCGAAACAAATTCATTTG |
| | AATGAAGGAATGCAAC |
| 539 | TTTTATTTTT |
| 540 | GGCTTCACTGATAAAGTGGAGAACCGCTTCACCAAAAGCTGTCCCTTAGGGGATTAG |
| | AACTTGAGTGAAGGTGGGCTGCTTGCATCAGCCTAATGTCGAGAAGTGCTTTCTTCG |
| | GAAAGTAACCCTCGAAACAAAGAAAGGAATGCAAC |
| 541 | GGCTTCACTGATAAAGTGGAGAACCGCTTCACTTAGAGTGAAGGTGGGCTGCTTGCA |
| | TCAGCCTAATGTCGAGAAGTGCTTTCTTCGGAAAGTAACCCTCGAAACAAATTCATT |
| | TGAATGAAGGAATGCAAC |
| 542 | TTTTATTTTT |
| 543 | GGCTTCACTGATAAAGTGGAGAACCGCTTCACCGAGTGAAGGTGGGCTGCTTGCATC |
| | AGCCTAATGTCGAGAAGTGCTTTCTTCGGAAAGTAACCCTCGAAACAAATTCATTTG |
| | AATGAAGGAATGCAAC |
| 544 | TTTTATTTTT |
| 545 | GGCTTCACTGATAAAGTGGAGAACCGCTTCACTTAGAGTGAAGGTGGGCTGCTTGCA |
| | TCAGCCTAATGTCGAGAAGTGCTTTCTTCGGAAAGTAACCCTCGAAACAAAGAAAG |
| | GAATGCAAC |
| 546 | GGCTTCACTGATAAAGTGGAGAACCGCTTCACCGAGTGAAGGTGGGCTGCTTGCATC |
| | AGCCTAATGTCGAGAAGTGCTTTCTTCGGAAAGTAACCCTCGAAACAAAGAAAGGA |
| | ATGCAAC |
| 547 | ACCGCTTCACTTAGAGTGAAGGTGGGCTGCTTGCATCAGCCTAATGTCGAGAAGTGC |
| | TTTCTTCGGAAAGTAACCCTCGAAACAAAGAAAGGAATGCAAC |
| 548 | ACCGCTTCACCGAGTGAAGGTGGGCTGCTTGCATCAGCCTAATGTCGAGAAGTGCTT |
| | TCTTCGGAAAGTAACCCTCGAAACAAAGAAAGGAATGCAAC |
| 549 | ACCGCTTCACTTAGAGTGAAGGTGGGCTGCTTGCATCAGCCTAATGTCGAGAAGTGC |
| | TTTCTTCGGAAAGTAACCCTCGAAACAAAGAAAGGAATGCAAC |
| 550 | TTTTATTTTT |
| 551 | ACCGCTTCACCGAGTGAAGGTGGGCTGCTTGCATCAGCCTAATGTCGAGAAGTGCTT |
| | TCTTCGGAAAGTAACCCTCGAAACAAAGAAAGGAATGCAAC |
| 552 | TTTTATTTTT |
| 553 | ACCGCTTCACGCTTCGGCAGTGAAGGTGGGCTGCTTGCATCAGCCTAATGTCGAGAA |
| | GTGCTTTCTTCGGAAAGTAACCCTCGAAACAAAGAAAGGAATGCAAC |
| 554 | ACCGCTTCACGCTTCGGCAGTGAAGGTGGGCTGCTTGCATCAGCCTAATGTCGAGAA |
| | GTGCTTTCTTCGGAAAGTAACCCTCGAAACAAAGAAAGGAATGCAAC |
| — | TTTTA |
| 555 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGAAAGGAATGCAAC |
| 556 | CCGCTTCACGCTTAGGCAGTGAAGGTGGGCTGCTTGCATCAGCCTAATGTCGAGAAG |
| | TGCTTTCTTCGGAAAGTAACCCTCGAAACAAAGAAAGGAATGCAAC |
| 557 | CCGCTTCACTCTTAGGAAGTGAAGGTGGGCTGCTTGCATCAGCCTAATGTCGAGAAG |
| | TGCTTTCTTCGGAAAGTAACCCTCGAAACAAAGAAAGGAATGCAAC |
| 558 | CCGCTTCACGTTTAGACAGTGAAGGTGGGCTGCTTGCATCAGCCTAATGTCGAGAAG |
| | TGCTTTCTTCGGAAAGTAACCCTCGAAACAAAGAAAGGAATGCAAC |
| 559 | GCCGCTTCACGCTTCGGCAGTGAAGGTGGGCTGCTTGCATCAGCCTAATGTCGAGAA |
| | GTGCTTTCTTCGGAAAGTAACCCTCGAAACAAAGAAAGGAATGCAAC |
| 560 | GGCCGCTTCACGCTTCGGCAGTGAAGGTGGGCTGCTTGCATCAGCCTAATGTCGAGA |
| | AGTGCTTTCTTCGGAAAGTAACCCTCGAAACAAAGAAAGGAATGCAAC |
| 561 | CGCTTCACGCTTCGGCAGTGAAGGTGGGCTGCTTGCATCAGCCTAATGTCGAGAAGT |
| | GCTTTCTTCGGAAAGTAACCCTCGAAACAAAGAAAGGAATGCAAC |
| 562 | GCTTCACGCTTCGGCAGTGAAGGTGGGCTGCTTGCATCAGCCTAATGTCGAGAAGTG |
| | CTTTCTTCGGAAAGTAACCCTCGAAACAAAGAAAGGAATGCAAC |
| 563 | GGCTTCACGCTTCGGCAGTGAAGGTGGGCTGCTTGCATCAGCCTAATGTCGAGAAGT |
| | GCTTTCTTCGGAAAGTAACCCTCGAAACAAAGAAAGGAATGCAAC |
| 564 | GCTTCACGCTTCGGCAGTGAAGGTGGGCTGCTTGCATCAGCCTAATGTCGAGAAGTG |
| | CTTTCTTCGGAAAGTAACCCTCGAAACAAAGAAAGGAATGCAAC |
| 550 | TTTTATTTTT |
| 565 | GGCTTCACGCTTCGGCAGTGAAGGTGGGCTGCTTGCATCAGCCTAATGTCGAGAAGT |
| | GCTTTCTTCGGAAAGTAACCCTCGAAACAAAGAAAGGAATGCAAC |
| 550 | TTTTATTTTT |
| 566 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGAAGGAATGCAAC |
| 567 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGAGGAATGCAAC |
| 568 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGGGAATGCAAC |
| 569 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGGAATGCAAC |
| 570 | CCGCTTCACGCTTCGGCAGTGAAGGTGGGCTGCTTGCATCAGCCTAATGTCGAGAAG |
| | TGCTTTCTTCGGAAAGTAACCCTCGAAACAAGGAATGCAAC |
| 571 | CCGCTTCACGCTTCGGCAGTGAAGGTGGGCTGCTTGCATCAGCCTAATGTCGAGAAG |
| | TGCTTTCTTCGGAAAGTAACCCTCGAAACAGGAATGCAAC |
| 572 | CCGCTTCACGCTTCGGCAGTGAAGGTGGGCTGCTTGCATCAGCCTAATGTCGAGAAG |
| | TGCTTTCTTCGGAAAGTAACCCTCGAAACGGAATGCAAC |
| 573 | CCGCTTCACGCTTCGGCAGTGAAGGTGGGCTGCTTGCATCAGCCTAATGTCGAGAAG |
| | TGCTTTCTTCGGAAAGTAACCCTCGAAAGGAATGCAAC |
| 574 | CCGCTTCACGCTTCGGCAGTGAAGGTGGGCTGCTTGCATCAGCCTAATGTCGAGAAG |
| | TGCTTTCTTCGGAAAGTAACCCTCGAAGGAATGCAAC |
| 575 | CCGCTTCACGCTTCGGCAGTGAAGGTGGGCTGCTTGCATCAGCCTAATGTCGAGAAG |
| | TGCTTTCTTCGGAAAGTAACCCTCGAGGAATGCAAC |
| 576 | CCGCTTCACGCTTCGGCAGTGAAGGTGGGCTGCTTGCATCAGCCTAATGTCGAGAAG |
| | TGCTTTCTTCGGAAAGTAACCCTCGAAACAAGAATGCAAC |
| 577 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGGAATGCAAC |
| 578 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGAATGCAAC |
| 579 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGAAAGAATGCAAC |
| 580 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGAGAATGCAAC |
| 581 | CCGCTTCACGCTTCGGCAGTGAAGGTGGGCTGCTTGCATCAGCCTAATGTCGAGAAG |
| | TGCTTTCTTCGGAAAGTAACCCTCGAAACAAAGAAATGCAAC |
| 582 | CCGCTTCACGCTTCGGCAGTGAAGGTGGGCTGCTTGCATCAGCCTAATGTCGAGAAG |
| | TGCTTTCTTCGGAAAGTAACCCTCGAAACAGAATGCAAC |
| 583 | CCGCTTCACGCTTCGGCAGTGAAGGTGGGCTGCTTGCATCAGCCTAATGTCGAGAAG |
| | TGCTTTCTTCGGAAAGTAACCCTCGAAACAAATGCAAC |
| 584 | CCGCTTCACGCTTCGGCAGTGAAGGTGGGCTGATTGCATCAGCCTAATGTCGAGAAG |
| | TGCTTTCTTCGGAAAGTAACCCTCGAAACAAAGAAAGGAATGCAAC |
| 585 | CCGCTTCACGCTTCGGCAGTGAAGGTAGGCTGCTTGCATCAGCCTAATGTCGAGAAG |
| | TGCTTTCTTCGGAAAGTAACCCTCGAAACAAAGAAAGGAATGCAAC |
| 586 | CCGCTTCACGCTTCGGCAGTGAAGGTGGGCTGCTTGCATCAGCCCAATGTCGAGAAG |
| | TGCTTTCTTCGGAAAGTAACCCTCGAAACAAAGAAAGGAATGCAAC |
| 587 | GGCTTCACGCTTCGGCAGTGAAGGTAGGCTGCTTGCATCAGCCTAATGTCGAGAAGT |
| | GCTTTCTTCGGAAAGTAACCCTCGAAACAAGGAATGCAAC |
| 588 | GGCTTCACGCTTCGGCAGTGAAGGTGGGCTGCTTGCATCAGCCCAATGTCGAGAAGT |
| | GCTTTCTTCGGAAAGTAACCCTCGAAACAAGGAATGCAAC |
| 589 | CCGCTTCACTCTTAGGAAGTGAAGGTGGGCTGATTGCATCAGCCTAATGTCGAGAAG |
| | TGCTTTCTTCGGAAAGTAACCCTCGAAACAAAGAAAGGAATGCAAC |
| 590 | CCGCTTCACGCTTCGGCAGTGAAGGTGGGCTGATTGCATCAGCCTAATGTCGAGAAG |
| | TGCTTTCTTCGGAAAGTAACCCTCGAAACAAAGGGAATGCAAC |
| 591 | CCGCTTCACGCTTCGGCAGTGAAGGTGGGCTGATTGCATCAGCCTAATGTCGAGAAG |
| | TGCTTTCTTCGGAAAGTAACCCTCGAAACAAAGGAATGCAAC |
| 592 | CCGCTTCACGCTTCGGCAGTGAAGGTGGGCTGATTGCATCAGCCTAATGTCGAGAAG |
| | TGCTTTCTTCGGAAAGTAACCCTCGAAACAAGGAATGCAAC |
| 593 | CCGCTTCACGCTTCGGCAGTGAAGGTGGGCTGATTGCATCAGCCTAATGTCGAGAAG |
| | TGCTTTCTTCGGAAAGTAACCCTCGAAACAAGAATGCAAC |
| 594 | CCGCTTCACGCTTCGGCAGTGAAGGTGGGCTGATTGCATCAGCCTAATGTCGAGAAG |
| | TGCTTTCTTCGGAAAGTAACCCTCGAAACAAAGGAATGCAAC |
| 595 | CCGCTTCACGCTTCGGCAGTGAAGGTGGGCTGATTGCATCAGCCTAATGTCGAGAAG |
| | TGCTTTCTTCGGAAAGTAACCCTCGAAACAAAGAATGCAAC |
| 596 | CCGCTTCACGCTTCGGCAGTGAAGGTGGGCTGATTGCATCAGCCTAATGTCGAGAAG |
| | TGCTTTCTTCGGAAAGTAACCCTCGAAACAGAATGCAAC |

TABLE 3
Exemplary list of guide RNA scaffold
fragment sequences.

| SEQ ID | Guide nucleic acid (NA) |
|---|---|
| NO | scaffold fragment sequence |
| 597 | CCGCTTCACGCTTCGGCAGTGAAGGTGGGC |
| 598 | CCGCTTCACTCTTAGGAAGTGAAGGTGGGC |
| 599 | GAAAGTAACCCTCGAAACAAAGAATGCAAC |
| 600 | AAGTAACCCTCGAAACAAAGGGAATGCAAC |
| 601 | AAAGTAACCCTCGAAACAAAGGAATGCAAC |

TABLE 4
Exemplary list of guide RNA spacer sequences.

| SEQ | | | |
|---|---|---|---|
| ID | | | |
| NO. | Rank | gRNA Spacer Sequence | Location |
| 700 | 1 | GGGGCATTTGAGTCACCTTTCTA | chr3: 129527721 |
| 701 | 2 | TCCAGAGGAAAGTGAGCAGTGGC | chr3: 129527766 |
| 702 | 3 | ATTCTGACGTGGACCCTCATGGC | chr3: 129526732 |
| 703 | 4 | AGAGAAGGAGCAGTTTGCCAGGT | chr3: 129527863 |
| 704 | 5 | TTCTCCCAGTCTCTCATCTCGTC | chr3: 129528274 |
| 705 | 6 | AAATAGGGACCATGGGCCTCTGT | chr3: 129528334 |
| 706 | 7 | GCACACGTGGCAGGGCTAAGGCA | chr3: 129526841 |
| 707 | 8 | CCACCCTGGGCGGTATGAGCCGG | chr3: 129529079 |
| 708 | 9 | GATAACATTGACAGGACAGGAGA | chr3: 129529195 |
| 709 | 10 | TGCAGGGCTGGCACTGAACACTG | chr3: 129529275 |
| 710 | 11 | GAGCAATATGCGCTTGTCTAATT | chr3: 129529313 |
| 711 | 12 | CAGCACCAAGCCTCTGTTTCCCT | chr3: 129529451 |
| 712 | 13 | CACTCTCAGGGGTCCTTCCGCCT | chr3: 129527026 |
| 713 | 14 | TTTGTGGTCCCTGTGCCCCCTCA | chr3: 129527179 |
| 714 | 15 | TGGTCCCTGTGCCCCCTCACCCC | chr3: 129527183 |
| 715 | 16 | AGTCACCTTTCTACACCAGTGAT | chr3: 129527731 |
| 716 | 17 | CCAGGTGGCTAGGTGGGGAAGAT | chr3: 129527846 |
| 717 | 18 | GGCCTTTGAGAGAAGGAGCAGTT | chr3: 129527871 |
| 718 | 19 | CAGGCCCCTGGGAATACAAAGAT | chr3: 129528038 |
| 719 | 20 | AGGCGCTTGGCCTGGTGATTATG | chr3: 129526787 |
| 720 | 21 | TAAGGGTCTGGGGGGGTCAGAAC | chr3: 129528610 |
| 721 | 22 | GAGGGCTTCTTTGCCACCCTGGG | chr3: 129529066 |
| 722 | 23 | TGGGGTGAGGGGGCACAGGGACC | chr3: 129527188 |
| 723 | 24 | AGGCCCTCGTGGCTGATTAGGCC | chr3: 129527569 |
| 724 | 25 | AGTCCTCCTCAGCCCCTGAGCTC | chr3: 129527987 |
| 725 | 26 | AGCCTCAGCTCAGTTTTCTTGCT | chr3: 129529344 |
| 726 | 27 | ATGAACGAACAAGAGAGTGAATT | chr3: 129528071 |
| 727 | 28 | TTGACTGAATATATGAGGGCTTT | chr3: 129529219 |
| 728 | 29 | TATTCCCAGGGGCCTGCAAATAA | chr3: 129528041 |
| 729 | 30 | TTTGCAGGCCCCTGGGAATACAA | chr3: 129528042 |

TABLE 5
List of gRNA spacer that can bind to Rhodopsin.

| SEQ | Guide nucleic acid spacer | | | | |
|---|---|---|---|---|---|
| ID | sequence (without | | | | |
| NO | scaffold) | Strand | Chromosome | Start | End |
| 730 | CCCAGTCATCTGCCCCCAAG | + | chr3 | 129527472 | 129527491 |
| 731 | TGGATCCTGAGTACCTCTCC | + | chr3 | 129528439 | 129528458 |
| 732 | ATTCTCCAGTCATTGGGTCT | + | chr3 | 129529586 | 129529605 |
| 733 | CTTTAGAATAATGTCTTGCA | + | chr3 | 129529759 | 129529778 |
| 734 | TTTAGAATAATGTCTTGCAT | + | chr3 | 129529760 | 129529779 |
| 735 | GAACGAACAAGAGAGTGAAT | + | chr3 | 129528077 | 129528096 |
| 736 | AGGGTCTGGGGGGGTCAGAA | + | chr3 | 129528616 | 129528635 |
| 737 | ATAATGTCTTGCATTTAACA | + | chr3 | 129529766 | 129529785 |
| 738 | AGGAAAACAGATGGGGTGCT | + | chr3 | 129529785 | 129529804 |
| 739 | GATGTGGCCAGGCAGCAACA | + | chr3 | 129529875 | 129529894 |
| 740 | AATATTGTCCCTTTCACTGT | + | chr3 | 129530199 | 129530218 |
| 741 | CATGAGGGTCCACGTCAGAA | + | chr3 | 129526734 | 129526753 |
| 742 | CAGACAGATCTGGGAATCCT | + | chr3 | 129526949 | 129526968 |
| 743 | AATCCTGGGTGGGAAGAGAG | + | chr3 | 129526963 | 129526982 |
| 744 | CCACAGTAGGTGCTCAATAC | + | chr3 | 129527293 | 129527312 |
| 745 | CCCAAGGCTCTGACTACTTT | + | chr3 | 129527486 | 129527505 |
| 746 | TACTTTCTTTCTCACGGTAC | + | chr3 | 129527500 | 129527519 |
| 747 | ATCGGCCCTCGTGGGGCCAC | + | chr3 | 129527536 | 129527555 |
| 748 | GCCTCCAGGGCCAGCCTCCC | + | chr3 | 129527620 | 129527639 |
| 749 | CCTCCCCTGCTCTGGTAGCC | + | chr3 | 129527672 | 129527691 |
| 750 | AGCCCCCTCCATCCTCCCTC | + | chr3 | 129527688 | 129527707 |
| 751 | CAAGCCACTGCTCACTTTCC | + | chr3 | 129527763 | 129527782 |
| 752 | TAAAGCCAGGTTCCCCGGCC | + | chr3 | 129527789 | 129527808 |
| 753 | TCTTCCCACATTTGAGTCCT | + | chr3 | 129527977 | 129527996 |
| 754 | CAGGGCTGTTTCTTTCCATC | + | chr3 | 129528021 | 129528040 |
| 755 | ACCAGAAACGGAAGCTGCAG | + | chr3 | 129528151 | 129528170 |
| 756 | GCGGGGATTAATATGATTAT | + | chr3 | 129528532 | 129528551 |
| 757 | GGGGTCAGAACCCAGAGTCA | + | chr3 | 129528626 | 129528645 |
| 758 | GATCGTGCTGGGCTTCCCCA | + | chr3 | 129528874 | 129528893 |
| 759 | TGGATACTTCGTCTTCGGGC | + | chr3 | 129529033 | 129529052 |
| 760 | AGAGTCCCGGGCTTGGCGGT | + | chr3 | 129529152 | 129529171 |
| 761 | TCCCTTGGAGCAGCTGTGCT | + | chr3 | 129529473 | 129529492 |
| 762 | GGCCCAAGCTCAGGGTGGGA | + | chr3 | 129529554 | 129529573 |
| 763 | ATCCTCTGCCTCCCCTCTCA | + | chr3 | 129529642 | 129529661 |
| 764 | TCCCCTCTCAGCCCCTGTCC | + | chr3 | 129529652 | 129529671 |
| 765 | GCAGTTCCTTTTTGCTTTAG | + | chr3 | 129529745 | 129529764 |
| 766 | GAGATGCAGGAGGAGACGCT | + | chr3 | 129529937 | 129529956 |
| 767 | AGCCTTGCCCTGTCTCCCCC | + | chr3 | 129530023 | 129530042 |
| 768 | CATTGTTGGGTGTTTGTTGC | + | chr3 | 129530088 | 129530107 |
| 769 | CAGAAGGTGGGTGTGCCACT | + | chr3 | 129530139 | 129530158 |
| 770 | GGGTCAGTCCCAGTTTACAA | + | chr3 | 129530180 | 129530199 |
| 771 | TTCCATCATTTCCTTCTTCT | + | chr3 | 129530305 | 129530324 |
| 772 | CAAAACATTGCACATTGCTT | + | chr3 | 129530334 | 129530353 |
| 773 | TTTCCACCACCTCTGCATTC | + | chr3 | 129530438 | 129530457 |
| 774 | TTCCTTCCCAACAAGGAACT | + | chr3 | 129530455 | 129530474 |
| 775 | CCACATTAGGATGCATTCTT | + | chr3 | 129530480 | 129530499 |
| 776 | AAACACACACACACACACAC | + | chr3 | 129530505 | 129530524 |
| 777 | TGTTTGTGGTCCCTGTGCCC | + | chr3 | 129527181 | 129527200 |
| 778 | CCAGTGATCTGCCCAAGCCA | + | chr3 | 129527750 | 129527769 |
| 779 | TGTCCAGAGGACATAGCACA | + | chr3 | 129528317 | 129528336 |
| 780 | TGCCCTTCTCCAATGCGACG | + | chr3 | 129528765 | 129528784 |
| 781 | TCACCGTCCAGCACAAGAAG | + | chr3 | 129528915 | 129528934 |
| 782 | CCTCTCTGCATGGATACTTC | + | chr3 | 129529023 | 129529042 |
| 783 | TTCACAGCAAGAAAACTGAG | + | chr3 | 129529339 | 129529358 |
| 784 | TCGGGCCATGTTTGCAGCAC | + | chr3 | 129529441 | 129529460 |
| 785 | GGGGGCTCAGCCCGGCCAGG | + | chr3 | 129526656 | 129526675 |
| 786 | CTCAGCCCGGCCAGGGAGGG | + | chr3 | 129526661 | 129526680 |
| 787 | AGTCTTGACCCAAGGCATCC | + | chr3 | 129526695 | 129526714 |
| 788 | TAATCACCAGGCCAAGCGCC | + | chr3 | 129526789 | 129526808 |
| 789 | GCAGGGAAGGGGGCACTCTT | + | chr3 | 129526924 | 129526943 |
| 790 | TCCTGGGTGGGAAGAGAGAC | + | chr3 | 129526965 | 129526984 |
| 791 | GGAAGAGAGACAGTGAGAGA | + | chr3 | 129526974 | 129526993 |
| 792 | GAGAGACAGTGAGAGAGAGA | + | chr3 | 129526978 | 129526997 |
| 793 | TGGCTGCGACTGAACTGTCC | + | chr3 | 129527138 | 129527157 |
| 794 | ACAGTAGGTGCTCAATACAC | + | chr3 | 129527295 | 129527314 |
| 795 | GTGTCATCATGTTGCTTGGA | + | chr3 | 129527409 | 129527428 |
| 796 | CACCTGGTACATGGCATTTG | + | chr3 | 129527553 | 129527572 |
| 797 | CATTTGAGTCACCTTTCTAC | + | chr3 | 129527729 | 129527748 |
| 798 | GGGCTGTTTCTTTCCATCTT | + | chr3 | 129528023 | 129528042 |
| 799 | CCTGGGCCCTAGGCTATGTG | + | chr3 | 129528125 | 129528144 |
| 800 | CTAGGCTATGTGTCTGGCAC | + | chr3 | 129528133 | 129528152 |
| 801 | CTGGATGACTCCAGAGGTAA | + | chr3 | 129528221 | 129528240 |
| 802 | AACGAACAGGTAAGGGGCTG | + | chr3 | 129528251 | 129528270 |
| 803 | AATAAACCAGAAAGTCTCTA | + | chr3 | 129528295 | 129528314 |
| 804 | CTTGGGACAGACAAGTCATG | + | chr3 | 129528387 | 129528406 |
| 805 | AGACAAGTCATGCAGAAGTT | + | chr3 | 129528395 | 129528414 |
| 806 | GGTCAGAACCCAGAGTCATC | + | chr3 | 129528628 | 129528647 |
| 807 | GGAGCAGCCACGGGTCAGCC | + | chr3 | 129528699 | 129528718 |
| 808 | CAGCCACGGGTCAGCCACAA | + | chr3 | 129528703 | 129528722 |
| 809 | TCCCCATCAACTTCCTCACG | + | chr3 | 129528888 | 129528907 |
| 810 | GTATGAGCCGGGTGTGGGTG | + | chr3 | 129529095 | 129529114 |
| 811 | GGGTGTGCAGGAGCCCGGGA | + | chr3 | 129529115 | 129529134 |
| 812 | GTGCAGGAGCCCGGGAGCAT | + | chr3 | 129529119 | 129529138 |
| 813 | AGTCCCGGGCTTGGCGGTGG | + | chr3 | 129529154 | 129529173 |
| 814 | GTCACACAGGGACGGGTGCA | + | chr3 | 129529394 | 129529413 |
| 815 | CTGAGGGAGAGCTGGGCAAG | + | chr3 | 129529514 | 129529533 |
| 816 | AGCCAGACCCCTCCTCTCTG | + | chr3 | 129529532 | 129529551 |
| 817 | CCCAAGCTCAGGGTGGGAAG | + | chr3 | 129529556 | 129529575 |
| 818 | GTGGATTTTCCATTCTCCAG | + | chr3 | 129529575 | 129529594 |
| 819 | TTCCCTGTGCTGGGCAATGG | + | chr3 | 129529605 | 129529624 |
| 820 | ATGGGCTCGGTCCCCTCTGG | + | chr3 | 129529621 | 129529640 |
| 821 | CGGTCCCCTCTGGCATCCTC | + | chr3 | 129529628 | 129529647 |
| 822 | GCTGCAGGGATAACAGATCC | + | chr3 | 129529802 | 129529821 |
| 823 | ACGGGGAGAGCTTACCGCCA | + | chr3 | 129529983 | 129530002 |
| 824 | TTTGTTGCATTCAATAATCA | + | chr3 | 129530100 | 129530119 |
| 825 | TGCCACTTACGGGTGGTTGT | + | chr3 | 129530152 | 129530171 |
| 826 | GGTGAGCAGGACAGATGTCT | + | chr3 | 129530282 | 129530301 |
| 827 | AAACATTGCACATTGCTTCA | + | chr3 | 129530336 | 129530355 |
| 828 | TCCACCACCTCTGCATTCCT | + | chr3 | 129530440 | 129530459 |
| 829 | CCAGGTGGGCTGCAGGGAAG | + | chr3 | 129526913 | 129526932 |
| 830 | TCCTTCCCCTGAAGCTTCCT | + | chr3 | 129527058 | 129527077 |
| 831 | GTGGTCACTGAGCGGCCGCA | + | chr3 | 129527329 | 129527348 |
| 832 | ACGGGGAGAGGGGGACCTGC | + | chr3 | 129527431 | 129527450 |
| 833 | CGGCCCTCGTGGGGCCACCT | + | chr3 | 129527538 | 129527557 |
| 834 | CCCCTGAAGGGTTCTGCCCC | + | chr3 | 129527654 | 129527673 |
| 835 | AAGCCAGGTTCCCCGGCCTA | + | chr3 | 129527791 | 129527810 |
| 836 | GAATCTGCTTCTTCCCACAT | + | chr3 | 129527968 | 129527987 |
| 837 | TCCTCCTGTCAGAGGAGTGT | + | chr3 | 129528196 | 129528215 |
| 838 | ACTCCAGAGGTAACTTGTGG | + | chr3 | 129528228 | 129528247 |
| 839 | CTGAGTACCTCTCCTCCCTG | + | chr3 | 129528445 | 129528464 |
| 840 | CCTGAGTGGCTGAGCTCAGG | + | chr3 | 129528658 | 129528677 |
| 841 | CTTCGTCTTCGGGCCCACAG | + | chr3 | 129529039 | 129529058 |
| 842 | GCTTCTTTGCCACCCTGGGC | + | chr3 | 129529074 | 129529093 |
| 843 | GGTCTGGGAGAGTCCCGGGC | + | chr3 | 129529144 | 129529163 |
| 844 | AATATGCGCTTGTCTAATTT | + | chr3 | 129529321 | 129529340 |
| 845 | CCCGCATCTATCTCGGGCCA | + | chr3 | 129529429 | 129529448 |
| 846 | AGCTGTGCTGAGTCAGACCC | + | chr3 | 129529484 | 129529503 |
| 847 | TTCCATTCTCCAGTCATTGG | + | chr3 | 129529582 | 129529601 |
| 848 | TTCATGGGGTGGTGAGCAGG | + | chr3 | 129530272 | 129530291 |
| 849 | CAGGCAGGGAGACGGGCACA | − | chr3 | 129526554 | 129526573 |
| 850 | ACGGGCACAAAACACAAATA | − | chr3 | 129526565 | 129526584 |
| 851 | CGGGCACAAAACACAAATAA | − | chr3 | 129526566 | 129526585 |
| 852 | GCTGTCAGAAGCACTATGCA | − | chr3 | 129526597 | 129526616 |
| 853 | CTGTCAGAAGCACTATGCAA | − | chr3 | 129526598 | 129526617 |
| 854 | CTGTGCCCCCTCACCCCACA | − | chr3 | 129527193 | 129527212 |
| 855 | TGTGCCCCCTCACCCCACAA | − | chr3 | 129527194 | 129527213 |
| 856 | TGTCTAATTTCACAGCAAGA | − | chr3 | 129529331 | 129529350 |
| 857 | ATGTCTTGCATTTAACAGGA | − | chr3 | 129529769 | 129529788 |
| 858 | ATCCCACTTAACAGAGAGGA | − | chr3 | 129529818 | 129529837 |
| 859 | CCTTCTTCTTCCTCTGGGCA | − | chr3 | 129530316 | 129530335 |
| 860 | CACACACACACACACACACA | − | chr3 | 129530549 | 129530568 |
| 861 | GACGGGCACAAAACACAAAT | − | chr3 | 129526564 | 129526583 |
| 862 | ATCACCAGGCCAAGCGCCTT | − | chr3 | 129526791 | 129526810 |
| 863 | CTGCTCACTTTCCTCTGGAT | − | chr3 | 129527770 | 129527789 |
| 864 | ATTCCCAGGGGCCTGCAAAT | − | chr3 | 129528046 | 129528065 |
| 865 | AGATGAGAGACTGGGAGAAT | − | chr3 | 129528278 | 129528297 |
| 866 | TTAGGATGCATTCTTCTGCT | − | chr3 | 129530485 | 129530504 |
| 867 | TAAAAAGCTTCCATGCTGTC | − | chr3 | 129526583 | 129526602 |
| 868 | TGGCCATGAGGGTCCACGTC | − | chr3 | 129526730 | 129526749 |
| 869 | AAGGGGGCACTCTTCTGAGC | − | chr3 | 129526930 | 129526949 |
| 870 | GGGCACTCTTCTGAGCAGAC | − | chr3 | 129526934 | 129526953 |
| 871 | AGCTCAAGCTCCAGCTTCTC | − | chr3 | 129527095 | 129527114 |
| 872 | GCACTTGCACAGGCCGCCCC | − | chr3 | 129527364 | 129527383 |
| 873 | CCAGGGCCAGCCTCCCCTTC | − | chr3 | 129527624 | 129527643 |
| 874 | ATTACAACTGCCCCCAGCCC | − | chr3 | 129527824 | 129527843 |
| 875 | AGGCCCAAACATGGCCTCCC | − | chr3 | 129527889 | 129527908 |
| 876 | TAGGCTATGTGTCTGGCACC | − | chr3 | 129528134 | 129528153 |
| 877 | CTCATGGAGCTCCTCCTGTC | − | chr3 | 129528186 | 129528205 |
| 878 | TGTGGGGACTGGATGACTCC | − | chr3 | 129528213 | 129528232 |
| 879 | AGAGACTGGGAGAATAAACC | − | chr3 | 129528283 | 129528302 |
| 880 | CAGAAAGTCTCTAGCTGTCC | − | chr3 | 129528302 | 129528321 |
| 881 | CTGTCCAGAGGACATAGCAC | − | chr3 | 129528316 | 129528335 |
| 882 | TATTTCAAACCCAGGCCACC | − | chr3 | 129528352 | 129528371 |
| 883 | CTGAGCTGGGACCTTGGGAC | − | chr3 | 129528375 | 129528394 |
| 884 | TTGGGACAGACAAGTCATGC | − | chr3 | 129528388 | 129528407 |
| 885 | ATGAACACCCCCAATCTCCC | − | chr3 | 129528550 | 129528569 |
| 886 | TATAAGGGTCTGGGGGGGTC | − | chr3 | 129528612 | 129528631 |
| 887 | GTCTGGGGGGGTCAGAACCC | − | chr3 | 129528619 | 129528638 |
| 888 | GCCACAGCCATGAATGGCAC | − | chr3 | 129528725 | 129528744 |
| 889 | CGTCACACAGGGACGGGTGC | − | chr3 | 129529393 | 129529412 |
| 890 | TGGAGCAGCTGTGCTGAGTC | − | chr3 | 129529478 | 129529497 |
| 891 | GAGGGAGAGCTGGGCAAGCC | − | chr3 | 129529516 | 129529535 |
| 892 | CTTGCATTTAACAGGAAAAC | − | chr3 | 129529773 | 129529792 |
| 893 | TGGGGTGCTGCAGGGATAAC | − | chr3 | 129529796 | 129529815 |
| 894 | GATAACAGATCCCACTTAAC | − | chr3 | 129529810 | 129529829 |
| 895 | TTGTTGCATTCAATAATCAC | − | chr3 | 129530101 | 129530120 |
| 896 | CAGATCACTCAGTTCTGGCC | − | chr3 | 129530120 | 129530139 |
| 897 | CATGGGGTGGTGAGCAGGAC | − | chr3 | 129530274 | 129530293 |
| 898 | CACGTGTGCCAAACGCTGTT | − | chr3 | 129526855 | 129526874 |
| 899 | GTGTCACCTTGGCCCCTCTT | − | chr3 | 129528483 | 129528502 |
| 900 | AAGCAGTTCCTTTTTGCTTT | − | chr3 | 129529743 | 129529762 |
| 901 | GAGATGCAGGAGGAGACGCT | − | chr3 | 129529937 | 129529956 |
| 902 | AGTTGTGGGTCAGTCTTGAC | − | chr3 | 129526684 | 129526703 |
| 903 | GCCTTAAACTACGAGAGGCC | − | chr3 | 129526806 | 129526825 |
| 904 | AAACTACGAGAGGCCCCATC | − | chr3 | 129526811 | 129526830 |
| 905 | TGTGCCAAACGCTGTTAGAC | − | chr3 | 129526859 | 129526878 |
| 906 | GCTGTTAGACCCAACACCAC | − | chr3 | 129526869 | 129526888 |
| 907 | GCCAGGTAGGGGGCTGGAGC | − | chr3 | 129526893 | 129526912 |
| 908 | GAGAGATTAAGGGATATTTC | − | chr3 | 129526992 | 129527011 |
| 909 | GGTCCCTGTGCCCCCTCACC | − | chr3 | 129527188 | 129527207 |
| 910 | TTCGTGTACCCTGTGTGTCC | − | chr3 | 129527248 | 129527267 |
| 911 | CAGCACTTGCACAGGCCGCC | − | chr3 | 129527362 | 129527381 |
| 912 | CACAGGCCGCCCCAGACACC | − | chr3 | 129527371 | 129527390 |
| 913 | GTGTTGGCCTCCATTTTCCC | − | chr3 | 129527453 | 129527472 |
| 914 | TTTCCCCCAGTCATCTGCCC | − | chr3 | 129527467 | 129527486 |
| 915 | CGTGGCTGATTAGGCCTCCC | − | chr3 | 129527580 | 129527599 |
| 916 | TTTCTACACCAGTGATCTGC | − | chr3 | 129527742 | 129527761 |
| 917 | CCCGGCCTAGCGTTCAAGAC | − | chr3 | 129527802 | 129527821 |
| 918 | AAGACCCATTACAACTGCCC | − | chr3 | 129527817 | 129527836 |
| 919 | CCATTACAACTGCCCCCAGC | − | chr3 | 129527822 | 129527841 |
| 920 | GCCCCCAGCCCAGATCTTCC | − | chr3 | 129527833 | 129527852 |
| 921 | CTGCTCCTTCTCTCAAAGGC | − | chr3 | 129527873 | 129527892 |
| 922 | AAAGGCCCAAACATGGCCTC | − | chr3 | 129527887 | 129527906 |
| 923 | GGCCTCCCAGACTGCAACCC | − | chr3 | 129527901 | 129527920 |
| 924 | TGGACGGAATCTGCTTCTTC | − | chr3 | 129527962 | 129527981 |
| 925 | TTCTTTCCATCTTTGTATTC | − | chr3 | 129528030 | 129528049 |
| 926 | AGAGGACATAGCACAGAGGC | − | chr3 | 129528322 | 129528341 |
| 927 | CCATGGTCCCTATTTCAAAC | − | chr3 | 129528342 | 129528361 |
| 928 | TAATATGATTATGAACACCC | − | chr3 | 129528540 | 129528559 |
| 929 | TTATGAACACCCCCAATCTC | − | chr3 | 129528548 | 129528567 |
| 930 | GGGTCTGGGGGGGTCAGAAC | − | chr3 | 129528617 | 129528636 |
| 931 | TACGCAGCCCCTTCGAGTAC | − | chr3 | 129528792 | 129528811 |
| 932 | GCTGATCGTGCTGGGCTTCC | − | chr3 | 129528871 | 129528890 |
| 933 | TGGATACTTCGTCTTCGGGC | − | chr3 | 129529033 | 129529052 |
| 934 | GCAGCTGTGCTGAGTCAGAC | − | chr3 | 129529482 | 129529501 |
| 935 | GACCCCTCCTCTCTGGGGGC | − | chr3 | 129529537 | 129529556 |
| 936 | TGCTGCAGGGATAACAGATC | − | chr3 | 129529801 | 129529820 |
| 937 | CCAGCCTTGCCCTGTCTCCC | − | chr3 | 129530021 | 129530040 |
| 938 | TCCAGGCTGCTGCCTCGGTC | − | chr3 | 129530046 | 129530065 |
| 939 | TGTTCTCTGCAGGGTCAGTC | − | chr3 | 129530169 | 129530188 |
| 940 | CTTTCACTGTTAGGAATGTC | − | chr3 | 129530209 | 129530228 |
| 941 | ATGGCTCCTAGGAGAGGCCC | − | chr3 | 129530355 | 129530374 |
| 942 | CACCACCTCTGCATTCCTTC | − | chr3 | 129530442 | 129530461 |
| 943 | TCCCAACAAGGAACTCTGCC | − | chr3 | 129530460 | 129530479 |
| 944 | AAAACACAAATAAAAAGCTT | − | chr3 | 129526573 | 129526592 |
| 945 | TCAGTCTTGACCCAAGGCAT | − | chr3 | 129526693 | 129526712 |
| 946 | CTCCTTCTGGCCATGAGGGT | − | chr3 | 129526723 | 129526742 |
| 947 | TTCCGTTCTCAGCTCAAGCT | − | chr3 | 129527085 | 129527104 |
| 948 | GGACCTGCCAGTGTTGGCCT | − | chr3 | 129527443 | 129527462 |
| 949 | CAGTGCCCTGTCTGCTGCCT | − | chr3 | 129527604 | 129527623 |
| 950 | CCCTGCTCTGGTAGCCCCCT | − | chr3 | 129527676 | 129527695 |
| 951 | CCCCTCCATCCTCCCTCCCT | − | chr3 | 129527691 | 129527710 |
| 952 | CCATCCTCCCTCCCTCCACT | − | chr3 | 129527696 | 129527715 |
| 953 | CAGGCAGTCAGGCCCTGTCT | − | chr3 | 129527922 | 129527941 |
| 954 | CTGGGCAGGGCTGTTTCTTT | − | chr3 | 129528016 | 129528035 |
| 955 | AACGAACAAGAGAGTGAATT | − | chr3 | 129528078 | 129528097 |
| 956 | CAAGAGAGTGAATTCCAATT | − | chr3 | 129528084 | 129528103 |
| 957 | AGTGTGGGGACTGGATGACT | − | chr3 | 129528211 | 129528230 |
| 958 | ACCAGAAAGTCTCTAGCTGT | − | chr3 | 129528300 | 129528319 |
| 959 | GGGTCAGAACCCAGAGTCAT | − | chr3 | 129528627 | 129528646 |
| 960 | TAACTTCTACGTGCCCTTCT | − | chr3 | 129528754 | 129528773 |
| 961 | GGCTGAGCCATGGCAGTTCT | − | chr3 | 129528826 | 129528845 |
| 962 | CTCACGCTCTACGTCACCGT | − | chr3 | 129528902 | 129528921 |
| 963 | TCCTGTCCTGTCAATGTTAT | − | chr3 | 129529197 | 129529216 |
| 964 | CAGGGTGGGAAGTGGATTTT | − | chr3 | 129529564 | 129529583 |
| 965 | GGAAGTGGATTTTCCATTCT | − | chr3 | 129529571 | 129529590 |
| 966 | CCCTGTCCTCAGGTGCCCCT | − | chr3 | 129529664 | 129529683 |
| 967 | TCCAGCCTCCCTGCCGCGTT | − | chr3 | 129529683 | 129529702 |
| 968 | CTAGGTCTCCTGGCTGTGAT | − | chr3 | 129529900 | 129529919 |
| 969 | TTGCCCTGTCTCCCCCATGT | − | chr3 | 129530027 | 129530046 |
| 970 | GCAGGACAGATGTCTGAATT | − | chr3 | 129530287 | 129530306 |
| 971 | GGACTGCCAATTCTGGGTTT | − | chr3 | 129530421 | 129530440 |
| 972 | TCGTGTCCCTTATGGGCCCA | + | chr3 | 129530616 | 129530635 |
| 973 | CAGAGCGCTAAGCAAATAAC | + | chr3 | 129530634 | 129530653 |
| 974 | TTCGTGTCCCTTATGGGCCC | − | chr3 | 129530615 | 129530634 |
| 975 | CAAAACTCCCTACCGGGTTC | − | chr3 | 129530567 | 129530586 |
| 976 | GATTCGTGTCCCTTATGGGC | − | chr3 | 129530613 | 129530632 |

TABLE 6
Human genomic region that can be targeted.

| SEQ | | | Relative |
|---|---|---|---|
| ID | | | location |
| NO | Human Genomic Region Sequence | Strand | from TSS |
| 977 | CCTTCAGACTGGAGTCCCCTGAAGGGTTCTGCCCCTCCCCT | + | Upstream |
| | GCTCTGGTAGCCCCCTCCATCCTCCCTCCCTCCACTCCATCT | | |
| | TTGGGGGCATTTGAGTCACCTTTCTACACCAGTGATCTGCC | | |
| | CAAGCCACTGCTCACTTTCCTCTGGATAAAGCCAGGTTCCC | | |
| | CGGCCTAGCGTTCAAGACCCATTACAACTGCCCCCAGCCCA | | |
| | GATCTTCCCCACCTAGCCACCTGGCAAACTGCTCCTTCTCTC | | |
| | AAAGGCCCAAACATGGCCTCCCAGACTGCAACCCCCAGGC | | |
| | AGTCAGGCCCTGTCTCCACAACCTCACAGCCACCCTGGACG | | |
| | GAATCTGCTTCTTCCCACATTTGAGTCCTCCTCAGCCCCTGA | | |
| | GCTCCTCTGGGCAGGGCTGTTTCTTTCCATCTTTGTATTCCC | | |
| | AGGGGCCTGCAAATAAATGTTTAATGAACGAACAAGAGAG | | |
| | TGAATTCCAATTCCATGCAACAAGGATTGGGCTCCTGGGCC | | |
| | CTAGGCTATGTGTCTGGCACCAGAAACGGAAGCTGCAGGT | | |
| | TGCAGCCCCTGCCCTCATGGAGCTCCTCCTGTCAGAGGAGT | | |
| | GTGGGGACTGGATGACTCCAGAGGTAACTTGTGGGGGAAC | | |
| | GAACAGGTAAGGGGCTGTGTGACGAGATGAGAGACTGGGA | | |
| | GAATAAACCAGAAAGTCTCTAGCTGTCCAGAGGACATAGC | | |
| | ACAGAGGCCCATGGTCCCTATTTCAAACCCAGGCCACCAG | | |
| | ACTGAGCTGGGACCTTGGGACAGACAAGTCATGCAGAAGT | | |
| | TAGGGGACCTTCTCCTCCCTTTTCCTGGATCCTGAGTACCTC | | |
| | TCCTCCCTGACCTCAGGCTTCCTCCTAGTGTCACCTTGGCCC | | |
| | CTCTTAGAAGCCAATTAGGCCCTCAGTTTCTGCAGCGGGGA | | |
| | TTAATATGATTATGAACACCCCCAATCTCCCAGATGCTGAT | | |
| | TCAGCCAGGAGCTTAGGAGGGGGAGGTCACTTTATAAGGG | | |
| | TCTGGGGGGGTCAGAACCC | | |
| 978 | TATGTGTCTGGCACCAGAAACGGAAGCTGCAGGTTGCAGC | + | Upstream |
| | CCCTGCCCTCATGGAGCTCCTCCTGTCAGAGGAGTGTGGGG | | |
| | ACTGGATGACTCCAGAGGTAACTTGTGGGGGAACGAACAG | | |
| | GTAAGGGGCTGTGTGACGAGATGAGAGACTGGGAGAATAA | | |
| | ACCAGAAAGTCTCTAGCTGTCCAGAGGACATAGCACAGAG | | |
| | GCCCATGGTCCCTATTTCAAACCCAGGCCACCAGACTGAGC | | |
| | TGGGACCTTGGGACAGACAAGTCATGCAGAAGTTAGGGGA | | |
| | CCTTCTCCTCCCTTTTCCTGGATCCTGAGTACCTCTCCTCCC | | |
| | TGACCTCAGGCTTCCTCCTAGTGTCACCTTGGCCCCTCTTAG | | |
| | AAGCCAATTAGGCCCTCAGTTTCTGCAGCGGGGATTAATAT | | |
| | GATTATGAACACCCCCAATCTCCCAGATGCTGATTCAGCCA | | |
| | GGAGCTTAGGAGGGGGAGGTCACTTTATAAGGGTCTGGGG | | |
| | GGGTCAGAACCC | | |
| 979 | AGAGTCATCCAGCTGGAGCCCTGAGTGGCTGAGCTCAGGC | + | Downstream |
| | CTTCGCAGCATTCTTGGGTGGGAGCAGCCACGGGTCAGCCA | | |
| | CAAGGGCCACAGCCATGAATGGCACAGAAGGCCCTAACTT | | |
| | CTACGTGCCCTTCTCCAATGCGACGGGTGTGGTACGCAGCC | | |
| | CCTTCGAGTACCCACAGTACTACCTGGCTGAGCCATGGCAG | | |
| | TTCTCCATGCTGGCCGCCTACATGTTTCTGCTGATCGTGCTG | | |
| | GGCTTCCCCATCAACTTCCTCACGCTCTACGTCACCGTCCA | | |
| | GCACAAGAAGCTGCGCACGCCTCTCAACTACATCCTGCTCA | | |
| | ACCTAGCCGTGGCTGACCTCTTCATGGTCCTAGGTGGCTTC | | |
| | ACCAGCACCCTCTACACCTCTCTGCATGGATACTTCGTCTTC | | |
| | GGGCCCACAGGATGCAATTTGGAGGGCTTCTTTGCCACCCT | | |
| | GGGCGGTATGAGCCGGGTGTGGGTGGGGTGTGCAGGAGCC | | |
| | CGGGAGCATGGAGGGGTCTGGGAGAGTCCCGGGCTTGGCG | | |
| | GTGGTGGCTGAGAGGCCTTCTCCCTTCTCCTGTCCTGTCAAT | | |
| | GTTATCCAAAGCCCTCATATATTCAGTCAACAAACACCATT | | |
| | CATGGTGATAGCCGGGCTGCTGTTTGTGCAGGGCTGGCACT | | |
| | GAACACTGCCTTGATCTTATTTGGAGCAATATGCGCTTGTC | | |
| | TAATTTCACAGCAAGAAAACTGAGCTGAGGCTCAAAGAAG | | |
| | TCAAGCGCCCTGCTGGGGCGTCACACAGGGACGGGTGCAG | | |
| | AGTTGAGTTGGAAGCCCGCATCTATCTCGGGCCATGTTTGC | | |
| | AGCACCAAGCCTCTGTTTCCCTTGGAGCAGCTGTGCTGAGT | | |
| | CAGACCCAGGCTGGGCACTGAGGGAGAGCTGGGCAAGCCA | | |
| | GACCCCTCCTCTCTGGGGGCCCAAGCTCAGGGTGGGAAGT | | |
| | GGATTTTCCATTCTCCAGTCATTGGGTCTTCCCTGTGCTGGG | | |
| | CAATGGGCTCGGTCCCCTCT | | |
| 980 | AGAGTCATCCAGCTGGAGCCCTGAGTGGCTGAGCTCAGGC | + | Downstream |
| | CTTCGCAGCATTCTTGGGTGGGAGCAGCCACGGGTCAGCCA | | |
| | CAAGGGCCACAGCCATGAATGGCACAGAAGGCCCTAACTT | | |
| | CTACGTGCCCTTCTCCAATGCGACGGGTGTGGTACGCAGCC | | |
| | CCTTCGAGTACCCACAGTACTACCTGGCTGAGCCATGGCAG | | |
| | TTCTCCATGCTGGCCGCCTACATGTTTCTGCTGATCGTGCTG | | |
| | GGCTTCCCCATCAACTTCCTCACGCTCTACGTCACCGTCCA | | |
| | GCACAAGAAGCTGCGCACGCCTCTCAACTACATCCTGCTCA | | |
| | ACCTAGCCGTGGCTGACCTCTTCATGGTCCTAGGTGGCTTC | | |
| | ACCAGCACCCTCTACACCTCTCTGCATGGATACTTCGTCTTC | | |
| | GGGCCCACAGGATGCAATTTGGAGGGCTTCTTTGCCACCCT | | |
| | GGGCGGTATGAGCCGGGTGTGGGTGGGGTGTGCAGGAGCC | | |
| | CGGGAGCAT | | |
| 981 | GGGTTCTGACCCCCCCAGACCCTTATAAAGTGACCTCCCCC | − | Upstream |
| | TCCTAAGCTCCTGGCTGAATCAGCATCTGGGAGATTGGGGG | | |
| | TGTTCATAATCATATTAATCCCCGCTGCAGAAACTGAGGGC | | |
| | CTAATTGGCTTCTAAGAGGGGCCAAGGTGACACTAGGAGG | | |
| | AAGCCTGAGGTCAGGGAGGAGAGGTACTCAGGATCCAGGA | | |
| | AAAGGGAGGAGAAGGTCCCCTAACTTCTGCATGACTTGTCT | | |
| | GTCCCAAGGTCCCAGCTCAGTCTGGTGGCCTGGGTTTGAAA | | |
| | TAGGGACCATGGGCCTCTGTGCTATGTCCTCTGGACAGCTA | | |
| | GAGACTTTCTGGTTTATTCTCCCAGTCTCTCATCTCGTCACA | | |
| | CAGCCCCTTACCTGTTCGTTCCCCCACAAGTTACCTCTGGA | | |
| | GTCATCCAGTCCCCACACTCCTCTGACAGGAGGAGCTCCAT | | |
| | GAGGGCAGGGGCTGCAACCTGCAGCTTCCGTTTCTGGTGCC | | |
| | AGACACATAGCCTAGGGCCCAGGAGCCCAATCCTTGTTGC | | |
| | ATGGAATTGGAATTCACTCTCTTGTTCGTTCATTAAACATTT | | |
| | ATTTGCAGGCCCCTGGGAATACAAAGATGGAAAGAAACAG | | |
| | CCCTGCCCAGAGGAGCTCAGGGGCTGAGGAGGACTCAAAT | | |
| | GTGGGAAGAAGCAGATTCCGTCCAGGGTGGCTGTGAGGTT | | |
| | GTGGAGACAGGGCCTGACTGCCTGGGGGTTGCAGTCTGGG | | |
| | AGGCCATGTTTGGGCCTTTGAGAGAAGGAGCAGTTTGCCA | | |
| | GGTGGCTAGGTGGGGAAGATCTGGGCTGGGGGCAGTTGTA | | |
| | ATGGGTCTTGAACGCTAGGCCGGGGAACCTGGCTTTATCCA | | |
| | GAGGAAAGTGAGCAGTGGCTTGGGCAGATCACTGGTGTAG | | |
| | AAAGGTGACTCAAATGCCCCCAAAGATGGAGTGGAGGGAG | | |
| | GGAGGATGGAGGGGGCTACCAGAGCAGGGGAGGGGCAGA | | |
| | ACCCTTCAGGGGACTCCAGTCTGAAGG | | |
| 982 | GGGTTCTGACCCCCCCAGACCCTTATAAAGTGACCTCCCCC | − | Upstream |
| | TCCTAAGCTCCTGGCTGAATCAGCATCTGGGAGATTGGGGG | | |
| | TGTTCATAATCATATTAATCCCCGCTGCAGAAACTGAGGGC | | |
| | CTAATTGGCTTCTAAGAGGGGCCAAGGTGACACTAGGAGG | | |
| | AAGCCTGAGGTCAGGGAGGAGAGGTACTCAGGATCCAGGA | | |
| | AAAGGGAGGAGAAGGTCCCCTAACTTCTGCATGACTTGTCT | | |
| | GTCCCAAGGTCCCAGCTCAGTCTGGTGGCCTGGGTTTGAAA | | |
| | TAGGGACCATGGGCCTCTGTGCTATGTCCTCTGGACAGCTA | | |
| | GAGACTTTCTGGTTTATTCTCCCAGTCTCTCATCTCGTCACA | | |
| | CAGCCCCTTACCTGTTCGTTCCCCCACAAGTTACCTCTGGA | | |
| | GTCATCCAGTCCCCACACTCCTCTGACAGGAGGAGCTCCAT | | |
| | GAGGGCAGGGGCTGCAACCTGCAGCTTCCGTTTCTGGTGCC | | |
| | AGACACATA | | |
| 983 | AGAGGGGACCGAGCCCATTGCCCAGCACAGGGAAGACCCA | − | Downstream |
| | ATGACTGGAGAATGGAAAATCCACTTCCCACCCTGAGCTTG | | |
| | GGCCCCCAGAGAGGAGGGGTCTGGCTTGCCCAGCTCTCCCT | | |
| | CAGTGCCCAGCCTGGGTCTGACTCAGCACAGCTGCTCCAAG | | |
| | GGAAACAGAGGCTTGGTGCTGCAAACATGGCCCGAGATAG | | |
| | ATGCGGGCTTCCAACTCAACTCTGCACCCGTCCCTGTGTGA | | |
| | CGCCCCAGCAGGGCGCTTGACTTCTTTGAGCCTCAGCTCAG | | |
| | TTTTCTTGCTGTGAAATTAGACAAGCGCATATTGCTCCAAA | | |
| | TAAGATCAAGGCAGTGTTCAGTGCCAGCCCTGCACAAACA | | |
| | GCAGCCCGGCTATCACCATGAATGGTGTTTGTTGACTGAAT | | |
| | ATATGAGGGCTTTGGATAACATTGACAGGACAGGAGAAGG | | |
| | GAGAAGGCCTCTCAGCCACCACCGCCAAGCCCGGGACTCT | | |
| | CCCAGACCCCTCCATGCTCCCGGGCTCCTGCACACCCCACC | | |
| | CACACCCGGCTCATACCGCCCAGGGTGGCAAAGAAGCCCT | | |
| | CCAAATTGCATCCTGTGGGCCCGAAGACGAAGTATCCATGC | | |
| | AGAGAGGTGTAGAGGGTGCTGGTGAAGCCACCTAGGACCA | | |
| | TGAAGAGGTCAGCCACGGCTAGGTTGAGCAGGATGTAGTT | | |
| | GAGAGGCGTGCGCAGCTTCTTGTGCTGGACGGTGACGTAG | | |
| | AGCGTGAGGAAGTTGATGGGGAAGCCCAGCACGATCAGCA | | |
| | GAAACATGTAGGCGGCCAGCATGGAGAACTGCCATGGCTC | | |
| | AGCCAGGTAGTACTGTGGGTACTCGAAGGGGCTGCGTACC | | |
| | ACACCCGTCGCATTGGAGAAGGGCACGTAGAAGTTAGGGC | | |
| | CTTCTGTGCCATTCATGGCTGTGGCCCTTGTGGCTGACCCGT | | |
| | GGCTGCTCCCACCCAAGAATGCTGCGAAGGCCTGAGCTCA | | |
| | GCCACTCAGGGCTCCAGCTGGATGACTCT | | |
| 984 | ATGCTCCCGGGCTCCTGCACACCCCACCCACACCCGGCTCA | − | Downstream |
| | TACCGCCCAGGGTGGCAAAGAAGCCCTCCAAATTGCATCCT | | |
| | GTGGGCCCGAAGACGAAGTATCCATGCAGAGAGGTGTAGA | | |
| | GGGTGCTGGTGAAGCCACCTAGGACCATGAAGAGGTCAGC | | |
| | CACGGCTAGGTTGAGCAGGATGTAGTTGAGAGGCGTGCGC | | |
| | AGCTTCTTGTGCTGGACGGTGACGTAGAGCGTGAGGAAGTT | | |
| | GATGGGGAAGCCCAGCACGATCAGCAGAAACATGTAGGCG | | |
| | GCCAGCATGGAGAACTGCCATGGCTCAGCCAGGTAGTACT | | |
| | GTGGGTACTCGAAGGGGCTGCGTACCACACCCGTCGCATTG | | |
| | GAGAAGGGCACGTAGAAGTTAGGGCCTTCTGTGCCATTCAT | | |
| | GGCTGTGGCCCTTGTGGCTGACCCGTGGCTGCTCCCACCCA | | |
| | AGAATGCTGCGAAGGCCTGAGCTCAGCCACTCAGGGCTCC | | |
| | AGCTGGATGACTCT | | |

### Embodiments

The following non-limiting embodiments provide illustrative examples of the invention, but do not limit the scope of the invention.

Embodiment 1. A system comprising:
a heterologous polypeptide comprising an actuator moiety, wherein the actuator moiety is for binding an endogenous target gene encoding a target protein in a cell, to decrease expression level of the target protein, and wherein the actuator moiety substantially lacks DNA cleavage activity;
a heterologous polynucleotide encoding a non-disease causing variant of the endogenous target gene that encodes the target protein;
wherein the endogenous target gene is associated with an ocular disease,
optionally wherein:
(1) the endogenous target gene comprises G-protein-coupled receptor (GPCR), further optionally wherein the GPCR comprises Rhodopsin; and/or
(2) the actuator moiety is configured to bind to a non-coding region of the endogenous target gene; and/or
(3) the endogenous target gene comprises a disease causing allele of the target protein; and/or
(4) the endogenous target gene comprises a non-disease causing allele of the target protein, further optionally wherein the non-disease causing variant is a wild type allele; and/or
(5) the heterologous polynucleotide is not integrated into the endogenous target gene; and/or
(6) the heterologous polypeptide is under control of a tissue-specific promoter; and/or
(7) the heterologous polynucleotide is under control of a constitutive promoter; and/or
(8) the heterologous polynucleotide and an additional heterologous polynucleotide encoding the heterologous polypeptide are part of the same vector; and/or
(9) the actuator moiety is a deactivated Cas (dCas) protein, further optionally wherein:
   (i) a size of the dCas protein is less than or equal to about 800 amino acids; and/or
   (ii) a size of the dCas protein is less than or equal to about 600 amino acids; and/or
   (iii) the dCas protein comprises a polynucleotide sequence exhibiting at least about 90% sequence identity to the polynucleotide sequence selected from Table 1; and/or
(10) the system further comprises a guide nucleic acid capable of forming a complex with the actuator moiety, wherein the complex binds the endogenous target gene, further optionally wherein:
   (i) the guide nucleic acid comprises a plurality of different guide nucleic acids capable of targeting different regions of the endogenous target gene; and/or
   (ii) the guide nucleic acid exhibits at least about 70% sequence identity to the polynucleotide sequence of any one of SEQ ID NOs. 700-976; and/or
(11) the actuator moiety is coupled to a transcriptional repressor; and/or
(12) the actuator moiety is fused to the transcriptional repressor; and/or
(13) the cell is a retinal cell, further optionally wherein the retinal cell is a retinal pigment epithelium (RPE) cell; and/or
(14) the actuator moiety is configured to bind to a domain of the endogenous target gene, wherein the domain is free of a nucleotide mutation that causes the ocular disease; and/or
(15) the expression level of the endogenous target gene encoding the target protein in the cell is decreased by at least about 15%, at least about 20%, at least about 30%, at least about 40%, at least about 50%, at least about 60%, at least about 70%, or at least about 80% as compared to that in a control cell that does not have the actuator moiety; and/or
(16) the heterologous polynucleotide is configured to yield a copy number of the non-disease causing variant of the endogenous target gene of at least about 20,000, at least about 50,000, at least about 100,000, at least about 500,000, at least about 1,000,000, or at least about 1,500,000; and/or
(17) the cell is in a retina tissue; and/or
(18) the cell is in a human retina tissue.

Embodiment 2. One or more polynucleotides encoding the system of any one of the preceding embodiments,
optionally wherein:
(1) the one or more polynucleotides comprise a single polynucleotide encoding at least the heterologous polypeptide and the heterologous polynucleotide, further optionally wherein:
   (i) the single polynucleotide further encodes the guide nucleic acid; and/or
   (ii) the single polynucleotide has a size of less than or equal to about 5 kilobases; and/or
   (iii) the single polynucleotide has a size of less than or equal to about 4.7 kilobases.

Embodiment 3. A method comprising administrating the system of any one of the preceding embodiments to a subject in need thereof, optionally wherein:
(1) the administrating comprises intravitreal injection or subretinal injection; and/or
(2) the subject has or is suspected of having retinitis pigmentosa 4 (RP4), further optionally wherein the method further comprises, prior to the administrating, determining that the subject has the RP4.

Embodiment 4. A method comprising:
(a) decreasing expression level of an endogenous target gene encoding a target protein in a cell via action of a heterologous polypeptide comprising an actuator moiety, wherein the actuator moiety is for binding the endogenous target gene, and wherein the actuator moiety substantially lacks DNA cleavage activity; and
(b) contacting the cell with a heterologous polynucleotide encoding a non-disease causing variant of the endogenous target gene,
wherein the endogenous target gene is associated with an ocular disease,
optionally wherein:
(1) the endogenous target gene encodes a G-protein-coupled receptor (GPCR), further optionally wherein the GPCR comprises Rhodopsin; and/or
(2) the actuator moiety is configured to bind to a non-coding region of the endogenous target gene; and/or
(3) the endogenous target gene comprises a disease causing allele of the target protein; and/or
(4) the endogenous target gene comprises a non-disease causing allele of the target protein; and/or
(5) the non-disease causing variant is a wild type allele; and/or
(6) the heterologous polynucleotide is not integrated into the endogenous target gene; and/or
(7) subsequent to (a) and (b), the cell exhibits a decreased amount of cytoplasmic Rhodopsin and an increased amount of membrane-bound rhodopsin, as compared to a control cell that has not been subjected to (a) and/or (b); and/or
(8) subsequent to (a) and (b), apoptosis propensity of the cell is reduced by at least about 0.1-fold, at least about 1-fold, at least about 5-fold, at least about 10-fold, at least about 50-fold, at least about 100-fold, or at least about 500-fold, as compared to that of a control cell that has not been subjected to (a) and/or (b); and/or
(9) subsequent to (a) and (b), a degree of endoplasmic reticulum (ER) stress in the cell is reduced by at least about 0.1-fold, at least about 1-fold, at least about 5-fold, at least about 10-fold, at least about 50-fold, at least about 100-fold, or at least about 500-fold, as compared to that of a control cell that has not been subjected to (a) and/or (b); and/or
(10) the heterologous polypeptide is under control of a tissue-specific promoter; and/or
(11) the heterologous polynucleotide is under control of a constitutive promoter; and/or
(12) the heterologous polynucleotide and an additional heterologous polynucleotide encoding the heterologous polypeptide are part of the same vector; and/or
(13) the actuator moiety is a deactivated Cas (dCas) protein, further optionally wherein:
   (i) a size of the dCas protein is less than or equal to about 800 amino acids; and/or
   (ii) a size of the dCas protein is less than or equal to about 600 amino acids; and/or
   (iii) the dCas protein comprises a polynucleotide sequence exhibiting at least about 90% sequence identity to the polynucleotide sequence selected from Table 1; and/or
(14) the decreasing in (a) is via action of a complex comprising the actuator moiety and a guide nucleic acid, wherein the complex binds the endogenous target gene, further optionally wherein:
   (i) the guide nucleic acid comprises a plurality of different guide nucleic acids capable of targeting different regions of the endogenous target gene; and/or
   (ii) the guide nucleic acid exhibits at least about 70% sequence identity to the polynucleotide sequence of any one of SEQ ID NOs. 700-976; and/or
(15) the actuator moiety is coupled to a transcriptional repressor; and/or
(16) the actuator moiety is fused to the transcriptional repressor; and/or
(17) the cell is a retinal cell, further optionally wherein the retinal cell is a retinal pigment epithelium (RPE) cell; and/or
(18) the actuator moiety is configured to bind to a domain of the endogenous target gene, wherein the domain is free of a nucleotide mutation that causes the ocular disease; and/or
(19) the expression level of the target protein in the cell is decreased by at least about 15%, at least about 20%, at least about 30%, at least about 40%, at least about 50%, at least about 60%, at least about 70%, or at least about 80% as compared to that in a control cell that does not have the actuator moiety; and/or
(20) the heterologous polynucleotide is configured to yield a copy number of the non-disease causing variant of the endogenous target gene at least about 15,000, at least about 50,000, at least about 100,000, at least about 110,000, at least about 120,000, at least about 130,000, at least about 140,000, or at least about 150,000.

Embodiment 5. A system comprising:
a heterologous polypeptide comprising an actuator moiety, wherein the actuator moiety is for binding an endogenous target gene encoding Rhodopsin in a cell, to decrease expression level of the Rhodopsin; and
a heterologous polynucleotide encoding a non-disease causing variant of the Rhodopsin,
wherein the heterologous polynucleotide is not integrated into the endogenous target gene,
optionally wherein:
(1) the actuator moiety is configured to bind to a non-coding region of the endogenous target gene; and/or
(2) the endogenous target gene comprises a disease causing allele of the Rhodopsin;
and/or
(3) the endogenous target gene comprises a non-disease causing allele of the Rhodopsin;

and/or
(4) the non-disease causing variant is a wild type allele; and/or
(5) the endogenous target gene and the heterologous polynucleotide sequence are under control of different promoters; and/or
(6) the heterologous polypeptide is under control of a tissue-specific promoter; and/or
(7) the heterologous polynucleotide is under control of a constitutive promoter; and/or
(8) the heterologous polynucleotide and an additional heterologous polynucleotide encoding the heterologous polypeptide are part of the same vector; and/or
(9) the actuator moiety substantially lacks DNA cleavage activity; and/or
(10) the actuator moiety is a deactivated Cas (dCas) protein, further optionally wherein:
   (i) a size of the dCas protein is less than or equal to about 800 amino acids; and/or
   (ii) a size of the dCas protein is less than or equal to about 600 amino acids; and/or
   (iii) the dCas protein comprises a polynucleotide sequence exhibiting at least about 90% sequence identity to the polynucleotide sequence selected from Table 1; and/or
(11) the system further comprises a guide nucleic acid capable of forming a complex with the actuator moiety, wherein the complex binds the endogenous target gene, further optionally wherein:
   (i) the guide nucleic acid comprises a plurality of different guide nucleic acids capable of targeting different regions of the endogenous target gene; and/or
   (ii) the guide nucleic acid exhibits at least about 70% sequence identity to the polynucleotide sequence of any one of SEQ ID NOs. 700-976; and/or
(12) the actuator moiety is coupled to a transcriptional repressor; and/or
(13) the actuator moiety is fused to the transcriptional repressor; and/or
(14) the cell is a retinal cell, further optionally wherein the retinal cell is a retinal pigment epithelium (RPE) cell; and/or
(15) the actuator moiety is configured to bind to a domain of the endogenous target gene, wherein the domain is free of a nucleotide mutation that causes the ocular disease; and/or
(16) the expression level of the endogenous target gene encoding Rhodopsin in the cell is decreased by at least about 15%, at least about 20%, at least about 30%, at least about 40%, at least about 50%, at least about 60%, at least about 70%, or at least about 80% as compared to that in a control that does not have the actuator moiety; and/or
(17) the heterologous polynucleotide is configured to yield a copy number of the non-disease causing variant of the endogenous target gene of at least about 20,000, at least about 50,000, at least about 100,000, at least about 500,000, at least about 1,000,000, or at least about 1,500,000.

Embodiment 6. One or more polynucleotides encoding the system of any one of the preceding embodiments,
optionally wherein:
(1) the one or more polynucleotides comprise a single polynucleotide encoding at least the heterologous polypeptide and the heterologous polynucleotide, further optionally wherein:
   (i) the single polynucleotide further encodes the guide nucleic acid; and/or
   (ii) the single polynucleotide has a size of less than or equal to about 5 kilobases; and/or
   (iii) the single polynucleotide has a size of less than or equal to about 4.7 kilobases.

Embodiment 7. A method comprising administrating the system of any one of the preceding embodiments to a subject in need thereof,
optionally wherein:
(1) the administrating comprises intravitreal injection or subretinal injection; and/or
(2) the subject has or is suspected of having retinitis pigmentosa 4 (RP4), further optionally wherein, prior to the administrating, determining that the subject has the RP4.

Embodiment 8. A method comprising:
(a) decreasing expression level of an endogenous target gene encoding Rhodopsin in a cell, via action of a heterologous polypeptide comprising an actuator moiety, wherein the actuator moiety is for binding the endogenous target gene; and
(b) contacting the cell with a heterologous polynucleotide encoding a non-disease variant allele of the Rhodopsin, wherein the heterologous polynucleotide is not integrated into the endogenous target gene,
optionally wherein:
(1) the actuator moiety is configured to bind to a non-coding region of the endogenous target gene; and/or
(2) the endogenous target gene comprises a disease causing allele of the Rhodopsin; and/or
(3) the endogenous target gene comprises a non-disease causing allele of the Rhodopsin; and/or
(4) the non-disease causing variant is a wild type allele; and/or
(5) the endogenous target gene and the heterologous polynucleotide sequence are under control of different promoters; and/or
(6) subsequent to (a) and (b), the cell exhibits a decreased amount of cytoplasmic Rhodopsin and an increased amount of membrane-bound rhodopsin, as compared to a control cell in absence of (a) and/or (b); and/or
(7) subsequent to (a) and (b), apoptosis propensity of the cell is reduced by at least about 0.1-fold, at least about 1-fold, at least about 5-fold, at least about 10-fold, at least about 50-fold, at least about 100-fold, or at least about 500-fold, as compared to that of a control cell in absence of (a) and/or (b); and/or
(8) subsequent to (a) and (b), a degree of endoplasmic reticulum (ER) stress in the cell is reduced by at least about 0.1-fold, at least about 1-fold, at least about 5-fold, at least about 10-fold, at least about 50-fold, at least about 100-fold, or at least about 500-fold, as compared to that of a control cell in absence of (a) and/or (b); and/or
(9) the heterologous polypeptide is under control of a tissue-specific promoter; and/or
(10) the heterologous polynucleotide is under control of a constitutive promoter; and/or
(11) the heterologous polynucleotide and an additional heterologous polynucleotide encoding the heterologous polypeptide are part of the same vector; and/or
(12) the actuator moiety substantially lacks DNA cleavage activity; and/or
(13) the actuator moiety is a deactivated Cas (dCas) protein, further optionally wherein:
   (i) a size of the dCas protein is less than or equal to about 800 amino acids; and/or
   (ii) a size of the dCas protein is less than or equal to about 600 amino acids; and/or
   (iii) the dCas protein comprises a polynucleotide sequence exhibiting at least about 90% sequence identity to the polynucleotide sequence selected from Table 1; and/or
(14) the decreasing in (a) is via action of a complex comprising the actuator moiety and a guide nucleic acid, wherein the complex binds the endogenous target gene, further optionally wherein:
   (i) the guide nucleic acid comprises a plurality of different guide nucleic acids capable of targeting different regions of the endogenous target gene; and/or
   (ii) the guide nucleic acid exhibits at least about 70% sequence identity to the polynucleotide sequence of any one of SEQ ID NOs. 700-976; and/or
(15) the actuator moiety is coupled to a transcriptional repressor; and/or
(16) the actuator moiety is fused to the transcriptional repressor; and/or
(17) the cell is a retinal cell, further optionally wherein the retinal cell is a retinal pigment epithelium (RPE) cell; and/or
(18) the expression level of the Rhodopsin in the cell is decreased by at least about 15%, at least about 20%, at least about 30%, at least about 40%, at least about 50%, at least about 60%, at least about 70%, or at least about 80% as compared to that in a control that does not have the actuator moiety; and/or
(19) the heterologous polynucleotide is configured to yield a copy number of the non-disease causing variant of the Rhodopsin of at least about 15,000, at least about 50,000, at least about 100,000, at least about 110,000, at least about 120,000, at least about 130,000, at least about 140,000, or at least about 150,000.

Embodiment 9. A system comprising:
a heterologous nucleic acid molecule exhibiting specific binding to a target polynucleotide sequence of a chromosomal gene encoding Rhodopsin, to decrease expression level of the Rhodopsin in a cell,
wherein the target polynucleotide sequence (i) is part of a non-coding region of the chromosomal gene, and (ii) exhibits at least about 70% sequence identity to the polynucleotide sequence of any one of SEQ ID NOs. 700-976,
optionally wherein:
(1) the target polynucleotide sequence exhibits at least about 80% sequence identity to the polynucleotide sequence of any one of SEQ ID NOs. 700-976; and/or
(2) the target polynucleotide sequence exhibits at least about 90% sequence identity to the polynucleotide sequence of any one of SEQ ID NOs. 700-976; and/or
(3) the target polynucleotide sequence exhibits at least about 70% sequence identity to the polynucleotide sequence of any one of SEQ ID NOs. 700-729; and/or
(4) the target polynucleotide sequence exhibits at least about 80% sequence identity to the polynucleotide sequence of any one of SEQ ID NOs. 700-729; and/or
(5) the target polynucleotide sequence exhibits at least about 90% sequence identity to the polynucleotide sequence of any one of SEQ ID NOs. 700-729; and/or
(6) the target polynucleotide sequence is free of a nucleotide mutation that causes an ocular diseases; and/or
(7) the target polynucleotide sequence has a length of at least about 15 nucleotides; and/or
(8) the heterologous nucleic acid molecule is a guide nucleic acid molecule capable of forming a complex with a Cas protein; and/or
(9) the Cas protein is a deactivated Cas (dCas) protein, further optionally wherein:
(i) a size of the dCas protein is less than or equal to about 800 amino acids; and/or
(ii) a size of the dCas protein is less than or equal to about 600 amino acids; and/or
(iii) the dCas protein comprises a polynucleotide sequence exhibiting at least about 90% sequence identity to the polynucleotide sequence selected from Table 1.

Embodiment 10. A method comprising:
a) contacting a cell with a vector comprising a heterologous nucleic acid molecule exhibiting specific binding to a target polynucleotide sequence of a chromosomal gene encoding Rhodopsin,
wherein the target polynucleotide sequence (i) is part of a non-coding region of the chromosomal gene, and (ii) exhibits at least about 70% sequence identity to the polynucleotide sequence of any one of SEQ ID NOs. 700-976;
b) decreasing expression level of the Rhodopsin in the cell,
optionally wherein:
(1) the target polynucleotide sequence exhibits at least about 80% sequence identity to the polynucleotide sequence of any one of SEQ ID NOs. 700-976; and/or
(2) the target polynucleotide sequence exhibits at least about 90% sequence identity to the polynucleotide sequence of any one of SEQ ID NOs. 700-976; and/or
(3) the target polynucleotide sequence exhibits at least about 70% sequence identity to the polynucleotide sequence of any one of SEQ ID NOs. 700-729; and/or
(4) the target polynucleotide sequence exhibits at least about 80% sequence identity to the polynucleotide sequence of any one of SEQ ID NOs. 700-729; and/or
(5) the target polynucleotide sequence exhibits at least about 90% sequence identity to the polynucleotide sequence of any one of SEQ ID NOs. 700-729; and/or
(6) the target polynucleotide sequence is free of a nucleotide mutation that causes an ocular diseases; and/or
(7) the target polynucleotide sequence has a length of at least about 15 nucleotides; and/or
(8) the heterologous nucleic acid molecule is a guide nucleic acid molecule capable of forming a complex with a Cas protein; and/or
(9) the Cas protein is a deactivated Cas (dCas) protein, further optionally wherein:
   (i) a size of the dCas protein is less than or equal to about 800 amino acids; and/or
   (ii) a size of the dCas protein is less than or equal to about 600 amino acids; and/or
   (iii) the dCas protein comprises a polynucleotide sequence exhibiting at least about 90% sequence identity to the polynucleotide sequence selected from Table 1.

It shall be understood that different aspects of the invention can be appreciated individually, collectively, or in combination with each other. Various aspects of the invention described herein may be applied to any of the particular applications disclosed herein. The compositions of matter disclosed herein in the composition section of the present disclosure may be utilized in the method section including methods of use and production disclosed herein, or vice versa.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A system comprising:
a heterologous polypeptide comprising an actuator moiety, wherein the actuator moiety is for binding an endogenous target gene encoding a target protein in a cell, to decrease expression level of the target protein, and wherein the actuator moiety substantially lacks DNA cleavage activity;
a heterologous polynucleotide encoding a non-disease causing variant of the endogenous target gene that encodes the target protein;
wherein the endogenous target gene is associated with an ocular disease.

2-73. (canceled)


SEQUENCE LISTING (984 sequences)

SEQ ID NO: 1 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 2 - protein, 500 aa, synthetic construct
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 3 - protein, 422 aa, synthetic construct
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 4 - protein, 509 aa, synthetic construct
MEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 5 - protein, 499 aa, synthetic construct
MNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 6 - protein, 509 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENA

SEQ ID NO: 7 - protein, 499 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFK

SEQ ID NO: 8 - protein, 509 aa, synthetic construct
MAKNTITKTLKLRIVRPYNSAEVEKIVADEKNNREKIALEKNKDKVNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 9 - protein, 498 aa, synthetic construct
MAKNTITKTLKLRIVRPYNSAEVEKIVADEKNNREKIALECKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 10 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 11 - skipped

SEQ ID NO: 12 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 13 - protein, 544 aa, unidentified
MNMSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 14 - protein, 586 aa, unidentified
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 15 - protein, 603 aa, unidentified
MKSFKLKLLPTDEQNVLLNEVFCKWASLCTRMASKGHDKERLAPPDSSGNYFNKTQLNQVNTDVTDHMGALEESASQKERAVEKVKRRLKLISDMLSEPNLRDVSQQKPTTFRPLEWVKEGLLKTKYHTVHYWQKECDKLTKQKERMEKTIEKIKKGKITFKPTKMSLHQNCFSLSFGKGTFSMRPFSDTKRGINLDMLTAPIQPAIGKNDGKSSLRSKEFIARNIENYIIFSIHSQLFGLSRSEELLLNAKKEELVAKRDAMLKKKSDSLSKKIKELEKIVGRKITDSERSEIMSQGGKLSSEKFSEDNSYLKTLKVLAKDIIGREELFRLKKYPIVIRKPLNERKKLKNLKPDEWEYYLQLSYDELEKKEFTPKTIMGIDRGLKHILAIAIYDPVQNKFVKNMLIPNPILGWKWKLRKIKRSIQHMERRIRAQQNAHVPENQLKKRLKSIENKIDYYYHNVSRQILNLAHDFKSAIVVEDLQNMKQHGRKKSKGLRGLNYALSNFDYGKIMGLVKYKAESENVPLLTVLPAGTSQNCAYCLLYGKEQGNYVRNNVNSKIGKCKLHGEIDADINAARTIAICYHKNINEPKPYGERKTFKRK

SEQ ID NO: 16 - protein, 424 aa, unidentified
MKYTKVMRYQIIKPLNAEWDELGMVLRDIQKETRAALNKTIQLCWEYQGFSADYKQIHGQYPKPKDVLGYTSMHGYAYDRLKNEFSKIASSNLSQTIKRAVDKWNSDLKEILRGDRSIPNFRKDCPIDIVKQSTKIQKCNDGYVLSLGLINREYKNELGRKNGVFDVLIKANDKTQQTILERIINGDYTYTASQIINHKNKWFINLTYQFETKETALDPNNVMGVDLGIVYPVYIAFNNSLHRYHIKGGEIERFRRQVEKRKRELLNQGKYCGDGRKGHGYATRTKSIESISDKIARFRDTCNHKYSRFIVDMALKHNCGIIQMEDLTGISKESTFLKNWTYYDLQQKIEYKAREAGIQVIKIEPQYTSQRCSKCGYIDKENRQEQATFKCIECGFKTNADYNAARNIAIPNIDKIIRKTLKMQ

SEQ ID NO: 17 - protein, 443 aa, Ruminococcus sp.
MTLLVKVVKIHLISEQFDKAGNRIDYEEVNKILWELQKQTREAKNKTVQLLWEWNNFSSDYVKASGIYPKAKDIFGYSSVHGQANKELRTKLALNSSNLSTTTMDVCKNFNTYKKEVWKGKRSVPSYKSDQPLDLHKDSIKLIYENNEFYVRLALLKKAEFAKYGFKDGFRFKMQVKDNSTKTILERCFDEVYKINASKLLYDQKKKKWKLNLSYSFDNKNISELDKEKILGVDVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 18 - protein, 497 aa, Syntrophomonas palmitatica
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 19 - protein, 497 aa, Clostridium novyi
MITVRKIKLTIMGDKDTRNSQYKWIRDEQYNQYRALNMGMTYLAVNDILYMNESGLEIRTIKDLKDCEKDIDKNKKEIEKLTARLEKEQNKKNSSSEKLDEIKYKISLVENKIEDYKLKIVELNKILEETQKERMDIQKEFKEKYVDDLYQVLDKIPFKHLDNKSLVTQRIKADIKSDKSNGLLKGERSIRNYKRNFPLMTRGRDLKFKYDDNDDIEIKWMEGIKFKVILGNRIKNSLELRHTLHKVIEGKYKICDSSLQFDKNNNLILNLTLDIPIDIVNKKVSGRVVGVDLGLKIPAYCALNDVEYIKKSIGRIDDFLKVRTQMQSRRRRLQIAIQSAKGGKGRVNKLQALERFAEKEKNFAKTYNHFLSSNIVKFAVSNQAEQINMELLSLKETQNKSILRNWSYYQLQTMIEYKAQREGIKVKYIDPYHTSQTCSKCGNYEEGQRESQADFICKKCGYKVNADYNAARNIAMSNKYITKKEESKYYKIKESMV

SEQ ID NO: 20 - protein, 495 aa, synthetic construct
MAKNTITKTLKLRIVRPYNSAEVEKIVADEKERRKQAGGTGELDGGFYKKLEKKHSEMFSFDRLNLLLNQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 21 - protein, 496 aa, synthetic construct
MEVQKTVMKTLSLRILRPLYSQEIEKEIKEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 22 - protein, 495 aa, synthetic construct
MAKNTITKTLKLRIVRPLYSQEIEKEIKEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 23 - protein, 451 aa, synthetic construct
MIKVYRYEIVKPLDLDWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 24 - protein, 485 aa, synthetic construct
MITVRKIKLTIMGDKDTRNSQYKWIRDEQYNQYRALNMGMTYLAVNDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 25 - protein, 462 aa, synthetic construct
MGESVKAIKLKILDMFLDPECTKQDDNWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 26 - protein, 453 aa, synthetic construct
MKYTKVMRYQIIKPLNAEWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 27 - protein, 460 aa, synthetic construct
MTLLVKVVKIHLISEQFDKAGNRIDYEEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 28 - protein, 529 aa, synthetic construct
MAKNTITKTLKLRIVRPYYSQEIEKIVAEEKNRREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 29 - protein, 529 aa, synthetic construct
MAKNTITKTLKLRIVRPYYSAEVEKIVAEEKNNREKIALDKNKDKVKEACSKHLKVAAYCTTQVERNACLFCKARKLDDKFYKKLRGQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 30 - protein, 529 aa, synthetic construct
MAKNTITKTLKLRIVRPYYSAEIEKIVADEKNRREKIALEKNKDKVKEACSKHLKVAAYCTTQVERNACLFCKARKLDDKFYQKLRKQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 31 - protein, 529 aa, synthetic construct
MAKNTITKTLKLRIVRPYNSQEVEKIVAEEKNRREKIALDKNKDKVKEACSKHLKVAAYCTTQVERNACLFCKARKLDDKFYQKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 32 - protein, 529 aa, synthetic construct
MAKNTITKTLKLRIVRPYNSQEVEKIVAEEKNNREKIALDKNKDKVKEACSKHLKVAAYCTTQVERNACLFCKARKLDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 33 - protein, 529 aa, synthetic construct
MAKNTITKTLKLRIVRPYNSQEVEKIVAEEKNNREKIALDKNKDKVKEACSKHLKVAAYCTTQVERNACLFCKARKLDDKFYKKLRGQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 34 - protein, 529 aa, synthetic construct
MAKNTITKTLKLRIVRPYYSAEVEKIVAEEKNNREKIALDKNKDKVKEACSKHLKVAAYCTTQVERNACLFCKARKLDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 35 - protein, 529 aa, synthetic construct
MAKNTITKTLKLRIVRPYNSAEIEKIVADEKNRREKIALDKNKDKVKEACSKHLKVAAYCTTQVERNACLFCKARKLDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 36 - protein, 529 aa, synthetic construct
MAKNTITKTLKLRIVRPYNSAEIEKIVADEKNRREKIALDKNKDKVKEACSKHLKVAAYCTTQVERNACLFCKARKLDDKFYKKLRGQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 37 - protein, 529 aa, synthetic construct
MAKNTITKTLKLRIVRPYNSQEIEKIVAEEKNRREKIALDKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 38 - protein, 529 aa, synthetic construct
MAKNTITKTLKLRIVRPYNSAEIEKIVAEEKNRREKIALDKNKDKVKEACSKHLKVAAYCTTQVERNACLFCKARKLDDKFYKKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 39 - protein, 529 aa, synthetic construct
MAKNTITKTLKLRIVRPYNSAEVEKIVAEEKNRREKIALDKNKDKVKEACSKHLKVAAYCTTQVERNACLFCKARKLDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 40 - protein, 529 aa, synthetic construct
MAKNTITKTLKLRIVRPYNSAEVEKIVADEKNRREKIALDKNKDKVKEACSKHLKVAAYCTTQVERNACLFCKARKLDDKFYKKLRKQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 41 - protein, 529 aa, synthetic construct
MAKNTITKTLKLRIVRPYYSAEIEKIVADEKNRREKIALDKNKDKVKEACSKHLKVAAYCTTQVERNACLFCKARKLDDKFYQKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 42 - protein, 529 aa, synthetic construct
MAKNTITKTLKLRIVRPYYSAEIEKIVAEEKNRREKIALEKNKDKVKEACSKHLKVAAYCTTQVERNACLFCKARKLDDKFYQKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 43 - protein, 529 aa, synthetic construct
MAKNTITKTLKLRIVRPYYSAEIEKIVAEEKNNREKIALDKNKDKVKEACSKHLKVAAYCTTQVERNACLFCKARKLDDKFYQKLRGQFPDAVFWQEISEIFRQLQKQAREIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 44 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAARLFKNAAIASGLRSKIKSNFRLKELKNMKSGLPTTKSDNFPIPLVKQKGGQYTGFKISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 45 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFKISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 46 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAGLFKNAAIASGLRSKIKSNFRLKELKNMKSGLPTTKSDNFPIPLVKQKGGQYTGFKISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 47 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAARLFKNAAIASGLRSKIKSNFRLKELKNMKSGLPTTKSDNFPIPLVKQKGGQYTGFRISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 48 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFRISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 49 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAGLFKNAAIASGLRSKIKSNFRLKELKNMKSGLPTTKSDNFPIPLVKQKGGQYTGFRISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 50 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAARLFKNAAIASGLRSKIKSNFRLKELKNMKSGLPTTKSDNFPIPLVKQKGGQYTGFSISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 51 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFSISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 52 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAGLFKNAAIASGLRSKIKSNFRLKELKNMKSGLPTTKSDNFPIPLVKQKGGQYTGFSISNHNSDFIIKIPFGRWQVKKEIDKYRPWEKFDFRQVQKSPKPISLLLSTQRRKRNKGWSKDEGTEAEIRKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 53 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLENFNKKMFARRRILLKKNRHKRGGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 54 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIEGGDLFHFNKKMFARRRILLKKNRHKRAGHGAKNKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 55 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIEGGDLENFNKKMFARRRILLKKNRHKRGGHGRDKKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 56 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIEGGDLENFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 57 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGRDKKLKPIEQLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 58 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLENFNKKMFARRRILLKKNRHKRAGHGRDKKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 59 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLEHFNKKMFARRRILLKKNRHKRKGHGAKNKLKPIETLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 60 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDGGDLEHFNKKMFARRRILLKKNRHKRKGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 61 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDGGDLFHFNKKMFARRRILLKKNRHKRAGHGAKNKLKPIETLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 62 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDGGDLEHFNKKMFARRRILLKKNRHKRKGHGAKNKLKPIETLTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 63 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSIDSNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 64 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSIDSNDLFHFNKKMFARRRILLKKNRHKRAGHGAAHKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 65 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSIDSNDLFKFNKKMFARRRILLKKNRHKRAGHGAAHKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 66 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDSNDLFKFNKKMFARRRILLKKNRHKRAGHGAAHKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 67 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSIDSNDLFKFNKKMFARRRILLKKNRHKRAGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 68 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 69 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIDSNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 70 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFKFNKKMFARRRILLKKNRHKRKGHGAAHKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 71 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLERFNKKMFARRRILLKKNRHKRKGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 72 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLERFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 73 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLEKFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 74 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLFHFNKKMFARRRILLKKNRHKRAGHGRKKKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 75 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLEKFNKKMFARRRILLKKNRHKRAGHGRKKKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 76 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSIKGGDLEKFNKKMFARRRILLKKNRHKRAGHGRKKKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 77 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWAKEIADFFIKNKVGTVQMEDLSTMKRKEDSYFNIRLRGFWPYYEMQNKIEFKLKQYGIEIRKVAPNNTSQLCSKCGHLNNYFNFEYRKKNKFPKFKCEKCNFKENAAYNAARNISTPDIKSTKERP

SEQ ID NO: 78 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 79 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWAKEIADFFIKNKVGTVQMEDLSTMKRKEDSYFNIRLRGFWPYYEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 80 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 81 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISTPDIKSTKERP

SEQ ID NO: 82 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLSTMKRKEDSYFNIRLRGFWPYYEMQNKIEFKLKQYGIEIRKVAPNNTSKTCSKCGHLNNYFNFEYRKKNKFPKFKCEKCNFKENAAYNAALNISNPKLKSTKERP

SEQ ID NO: 83 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 84 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPKFKCEKCNFKENAAYNAARNISTPDIKSTKERP

SEQ ID NO: 85 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWSRYIADFFIKNKVGTVQMEDLESMKRKEDSYFNIRLRGFWPYYEMQNKIEFKLKQYGIKIRKVAPNNTSQRCSKCGHLNNYFNFEYRKKNKFPHFKCEKCNFKANAAYNAARNISNPNIKSTKERP

SEQ ID NO: 86 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACYIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 87 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACYIADFFIKNKVGTVQMENLESMKRKEDSYFNIRLRGFWPYAEMQNKIEFKLKQYGIEIRKVAPNNTSQRCSKCGHLNNYFNFEYRKKNKFPHFKCEKCNFKRNAAYNAARNISNPKLKSTKERP

SEQ ID NO: 88 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACYIADFFIKNKVGTVQMEDLESMKRKEDSYFNIRLRGFWPYAEMQNKIEFKLKQYGIEIRKVAPNNTSKTCSKCGHLNNYFNFEYRKKNKFPHFKCEKCNFKENAAYNAARNISNPNIKSTKERP

SEQ ID NO: 89 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWARYIADFFIKNKVGTVQMEDLESMKRKEDSYFNIRLRGFWPYYEMQNKIEFKLKQYGIKIRKVAPNNTSKTCSKCGHLNNYFNFEYRKKNKFPHFKCEKCNFKENAAYNAALNISNPKLKSTKERP

SEQ ID NO: 90 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQRCSKCGHLNNYFNFEYRKKNKFPHFKCEKCNFKRNAAYNAARNISNPNIKSTKERP

SEQ ID NO: 91 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 92 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKRNAAYNAARNISNPNIKSTKERP

SEQ ID NO: 93 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWANRIADFFIKNKVGTVQMENLESMKRKEDSYFNIRLRGFWPYAEMQNKIEFKLKQYGIKIRKVAPNNTSQRCSKCGHLNNYFNFEYRKKNKFPHFKCEKCNFKRNAAYNAAKNISNPKLKSTKERP

SEQ ID NO: 94 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQRCSKCGHLNNYFNFEYRKKNKFPHFKCEKCNFKRNAAYNAAKNISNPKLKSTKERP

SEQ ID NO: 95 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIKIRKVAPNNTSQRCSKCGHLNNYFNFEYRKKNKFPHFKCEKCNFKRNAAYNAAKNISNPKLKSTKERP

SEQ ID NO: 96 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWANRIADFFIKNKVGTVQMENLESMKRKEDSYFNIRLRGFWPYAEMQNKIEFKLKQYGIKIRKVAPNNTSQRCSKCGHLNNYFNFEYRKKNKFPHFKCEKCNFKENAAYNAALNISNPKLKSTKERP

SEQ ID NO: 97 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWANRIADFFIKNKVGTVQMENLESMKRKEDSYFNIRLRGFWPYAEMQNKIEFKLKQYGIEIRKVAPNNTSQRCSKCGHLNNYFNFEYRKKNKFPHFKCEKCNFKRNAAYNAALNISNPKLKSTKERP

SEQ ID NO: 98 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWSRFIADFFIKNKVGTVQMEDLESMKRKEDSYFNIRLRGFWPYYEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 99 - protein, 514 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIDVQLYSKEYSKDSGKGTHRYFLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 100 - protein, 514 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIASLSLLSNPAKQEMNVKRKISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 101 - protein, 540 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPIYERKPNRSIVGGLAVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISAAYNAARNLSTPDIEKFVAKATKGINLPEK

SEQ ID NO: 102 - protein, 590 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPTHETVLDPDKVMGVALGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNAAYNAAKNISTPYIDQIIMEKCLELGIPYRSITCKECGHIQASGNTCEVCGSTNILKPKK

SEQ ID NO: 103 - protein, 522 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPQTRVLDLNKIMGIALGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANAAYNAARNIAIPNIDKIIAESIK

SEQ ID NO: 104 - protein, 532 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPIDIVNKKVSGRVVGVALGLKIPAYCALNDVEYIKKSIGRIDDFLKVRTQMQSRRRRLQIAIQSAKGGKGRVNKLQALERFAEKEKNFAKTYNHFLSSNIVKFAVSNQAEQINMELLSLKETQNKSILRNWSYYQLQTMIEYKAQREGIKVKYIDPYHTSQTCSKCGNYEEGQRESQADFICKKCGYKVNAAYNAARNIAMSNKYITKKEESKYYKIKESMV

SEQ ID NO: 105 - protein, 524 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVETKETALDPNNVMGVALGIVYPVYIAFNNSLHRYHIKGGEIERFRRQVEKRKRELLNQGKYCGDGRKGHGYATRTKSIESISDKIARFRDTCNHKYSRFIVDMALKHNCGIIQMEDLTGISKESTFLKNWTYYDLQQKIEYKAREAGIQVIKIEPQYTSQRCSKCGYIDKENRQEQATFKCIECGFKTNAAYNAARNIAIPNIDKIIRKTLKMQ

SEQ ID NO: 106 - protein, 540 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVNRSIVGGLAVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISAAYNAARNLSTPDIEKFVAKATKGINLPEK

SEQ ID NO: 107 - protein, 590 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPDKVMGVALGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNAAYNAAKNISTPYIDQIIMEKCLELGIPYRSITCKECGHIQASGNTCEVCGSTNILKPKK

SEQ ID NO: 108 - protein, 523 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDLNKIMGIALGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANAAYNAARNIAIPNIDKIIAESIK

SEQ ID NO: 109 - protein, 524 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPNNVMGVALGIVYPVYIAFNNSLHRYHIKGGEIERFRRQVEKRKRELLNQGKYCGDGRKGHGYATRTKSIESISDKIARFRDTCNHKYSRFIVDMALKHNCGIIQMEDLTGISKESTFLKNWTYYDLQQKIEYKAREAGIQVIKIEPQYTSQRCSKCGYIDKENRQEQATFKCIECGFKTNAAYNAARNIAIPNIDKIIRKTLKMQ

SEQ ID NO: 110 - protein, 535 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDKEKILGVAVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNAAYNASQNIGIKNIDKIIEKDLQKQESEVQVNENK

SEQ ID NO: 111 - protein, 517 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 112 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 113 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 114 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 115 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 116 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 117 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 118 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 119 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 120 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 121 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 122 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 123 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 124 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 125 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 126 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 127 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 128 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 129 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 130 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 131 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 132 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 133 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 134 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 135 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 136 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 137 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 138 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 139 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 140 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 141 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 142 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 143 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 144 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 145 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 146 - protein, 495 aa, synthetic construct; identical to SEQ ID NO: 12

SEQ ID NO: 147 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 148 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 149 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 150 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 151 - protein, 483 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 152 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 153 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 154 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 155 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 156 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 157 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 158 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 159 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 160 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 161 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 162 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 163 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 164 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 165 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 166 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 167 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 168 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 169 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 170 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 171 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 172 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 173 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 174 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 175 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 176 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 177 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 178 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 179 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 180 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 181 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 182 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 183 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 184 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISTPDIKSTKERP

SEQ ID NO: 185 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNISNPNIKSTKERP

SEQ ID NO: 186 - protein, 495 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 187 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNI

SEQ ID NO: 188 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 189 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNI

SEQ ID NO: 190 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQRCSKCGHLNNYFNFEYRKKNKFPHFKCEKCNFKENAAYNAARNI

SEQ ID NO: 191 - protein, 483 aa, synthetic construct
MAKNTITKTLKLRIVRPYNSQEIEKIVAEEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNI

SEQ ID NO: 192 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNSFSRYSISDNDLFKFNKKMFARRRILLKKNRHKRKGHGAKNKLKPITELTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 193 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 194 - protein, 495 aa, synthetic construct
MAKNTITKTLKLRIVRPYNSAEIEKIVADEKERRKQAGGTGELDDKFYKKLRKQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 195 - protein, 495 aa, synthetic construct
MAKNTITKTLKLRIVRPYNSAEIEKIVADEKERRKQAGGTGELDDKFYKKLRKQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 196 - protein, 495 aa, synthetic construct
MAKNTITKTLKLRIVRPYNSAEIEKIVADEKERRKQAGGTGELDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 197 - protein, 495 aa, synthetic construct
MAKNTITKTLKLRIVRPYNSAEIEKIVADEKERRKQAGGTGELDDKFYKKLRKQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPKFKCEKCNFKENAAYNAALNISNPDIKSTKERP

SEQ ID NO: 198 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMEDLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 199 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPKLKSTKERP

SEQ ID NO: 200 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPDIKSTKERP

SEQ ID NO: 201 - protein, 495 aa, synthetic construct
MAKNTITKTLKLRIVRPYNSAEVEKIVADEKERRKQAGGTGELDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSQLCSKCGHLNNYFNFEYRKKNKFPHFKCEKCNFKENAAYNAALNISNPDIKSTKERP

SEQ ID NO: 202-499 - skipped (298 entries)

SEQ ID NO: 500 - DNA, 159 bp, synthetic construct
  source 1..159: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 501 - DNA, 139 bp, synthetic construct
  source 1..139: mol_type = other DNA
gaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 502 - DNA, 138 bp, synthetic construct
  source 1..138: mol_type = other DNA
aaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 503 - DNA, 137 bp, synthetic construct
  source 1..137: mol_type = other DNA
accgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 504 - DNA, 136 bp, synthetic construct
  source 1..136: mol_type = other DNA
ccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 505 - DNA, 151 bp, synthetic construct
  source 1..151: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaagcaataaggaatgcaac

SEQ ID NO: 506 - DNA, 148 bp, synthetic construct
  source 1..148: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaagaaaggaatgcaac

SEQ ID NO: 507 - DNA, 157 bp, synthetic construct
  source 1..157: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaatcttcggattaaggaatgcaac

SEQ ID NO: 508 - DNA, 153 bp, synthetic construct
  source 1..153: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattgcaaaaggaatgcaac

SEQ ID NO: 509 - DNA, 153 bp, synthetic construct
  source 1..153: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcgttaaggaatgcaac

SEQ ID NO: 510 - DNA, 151 bp, synthetic construct
  source 1..151: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaatgcaaaggaatgcaac

SEQ ID NO: 511 - DNA, 153 bp, synthetic construct
  source 1..153: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaagcaattaaggaatgcaac

SEQ ID NO: 512 - DNA, 149 bp, synthetic construct
  source 1..149: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaagaaaggaatgcaac

SEQ ID NO: 513 - DNA, 132 bp, synthetic construct
  source 1..132: mol_type = other DNA
ggcttcactgataaagtggagaaccgcttcacttagagtgaaggtgggctgcttgcatcagcctaatgtcgagaagtgctttcttcggaaagtaaccctcgaaacaaattcatttgaatgaaggaatgcaac

SEQ ID NO: 514 - DNA, 132 bp, synthetic construct
  source 1..132: mol_type = other DNA
ggcttcactgataaagtggagaaccgcttcacttcgagtgaaggtgggctgcttgcatcagcctaatgtcgagaagtgctttcttcggaaagtaaccctcgaaacaaattcatttgaatgaaggaatgcaac

SEQ ID NO: 515 - DNA, 131 bp, synthetic construct
  source 1..131: mol_type = other DNA
ggcttcactgataaagtggagaaccgcttcacttcggtgaaggtgggctgcttgcatcagcctaatgtcgagaagtgctttcttcggaaagtaaccctcgaaacaaattcatttgaatgaaggaatgcaac

SEQ ID NO: 516 - DNA, 134 bp, synthetic construct
  source 1..134: mol_type = other DNA
ggcttcactgataaagtggagaaccgcttcaccttaggagtgaaggtgggctgcttgcatcagcctaatgtcgagaagtgctttcttcggaaagtaaccctcgaaacaaattcatttgaatgaaggaatgcaac

SEQ ID NO: 517 - DNA, 134 bp, synthetic construct
  source 1..134: mol_type = other DNA
ggcttcactgataaagtggagaaccgcttcaccttcggagtgaaggtgggctgcttgcatcagcctaatgtcgagaagtgctttcttcggaaagtaaccctcgaaacaaattcatttgaatgaaggaatgcaac

SEQ ID NO: 518 - DNA, 136 bp, synthetic construct
  source 1..136: mol_type = other DNA
ggcttcactgataaagtggagaaccgcttcacgcttcggcagtgaaggtgggctgcttgcatcagcctaatgtcgagaagtgctttcttcggaaagtaaccctcgaaacaaattcatttgaatgaaggaatgcaac

SEQ ID NO: 519 - DNA, 135 bp, synthetic construct
  source 1..135: mol_type = other DNA
accgcttcaccaaaagctgtccttagggattagaacttgagtgaaggtgggctgcttgcatcagcctaatgtcgagaagtgctttcttcggaaagtaaccctcgaaacaaattcatttgaatgaaggaatgcaac

SEQ ID NO: 520 - DNA, 133 bp, synthetic construct
  source 1..133: mol_type = other DNA
accgcttcaccaaaagctgtcttaggattagaacttgagtgaaggtgggctgcttgcatcagcctaatgtcgagaagtgctttcttcggaaagtaaccctcgaaacaaattcatttgaatgaaggaatgcaac

SEQ ID NO: 521 - DNA, 131 bp, synthetic construct
  source 1..131: mol_type = other DNA
accgcttcaccaaaagctgtttagattagaacttgagtgaaggtgggctgcttgcatcagcctaatgtcgagaagtgctttcttcggaaagtaaccctcgaaacaaattcatttgaatgaaggaatgcaac

SEQ ID NO: 522 - DNA, 129 bp, synthetic construct
  source 1..129: mol_type = other DNA
accgcttcaccaaaagctgttagttagaacttgagtgaaggtgggctgcttgcatcagcctaatgtcgagaagtgctttcttcggaaagtaaccctcgaaacaaattcatttgaatgaaggaatgcaac

SEQ ID NO: 523 - DNA, 126 bp, synthetic construct
  source 1..126: mol_type = other DNA
accgcttcaccaaaagctttagagaacttgagtgaaggtgggctgcttgcatcagcctaatgtcgagaagtgctttcttcggaaagtaaccctcgaaacaaattcatttgaatgaaggaatgcaac

SEQ ID NO: 524 - DNA, 124 bp, synthetic construct
  source 1..124: mol_type = other DNA
accgcttcaccaaaagcttcggcacttgagtgaaggtgggctgcttgcatcagcctaatgtcgagaagtgctttcttcggaaagtaaccctcgaaacaaattcatttgaatgaaggaatgcaac

SEQ ID NO: 525 - DNA, 122 bp, synthetic construct
  source 1..122: mol_type = other DNA
accgcttcaccaaaagttcgcacttgagtgaaggtgggctgcttgcatcagcctaatgtcgagaagtgctttcttcggaaagtaaccctcgaaacaaattcatttgaatgaaggaatgcaac

SEQ ID NO: 526 - DNA, 120 bp, synthetic construct
  source 1..120: mol_type = other DNA
accgcttcaccaaaattcgtcttgagtgaaggtgggctgcttgcatcagcctaatgtcgagaagtgctttcttcggaaagtaaccctcgaaacaaattcatttgaatgaaggaatgcaac

SEQ ID NO: 527 - DNA, 118 bp, synthetic construct
  source 1..118: mol_type = other DNA
accgcttcaccaagttcgcttgagtgaaggtgggctgcttgcatcagcctaatgtcgagaagtgctttcttcggaaagtaaccctcgaaacaaattcatttgaatgaaggaatgcaac

SEQ ID NO: 528 - DNA, 116 bp, synthetic construct
  source 1..116: mol_type = other DNA
accgcttcaccaattcgttgagtgaaggtgggctgcttgcatcagcctaatgtcgagaagtgctttcttcggaaagtaaccctcgaaacaaattcatttgaatgaaggaatgcaac

SEQ ID NO: 529 - DNA, 114 bp, synthetic construct
  source 1..114: mol_type = other DNA
accgcttcaccattcgtgagtgaaggtgggctgcttgcatcagcctaatgtcgagaagtgctttcttcggaaagtaaccctcgaaacaaattcatttgaatgaaggaatgcaac

SEQ ID NO: 530 - DNA, 159 bp, synthetic construct; identical to SEQ ID NO: 500
  source 1..159: mol_type = other DNA

SEQ ID NO: 531 - skipped

SEQ ID NO: 532 - DNA, 159 bp, synthetic construct; identical to SEQ ID NO: 500
  source 1..159: mol_type = other DNA

SEQ ID NO: 533 - skipped

SEQ ID NO: 534 - DNA, 159 bp, synthetic construct; identical to SEQ ID NO: 500
  source 1..159: mol_type = other DNA

SEQ ID NO: 535 - skipped

SEQ ID NO: 536 - DNA, 159 bp, synthetic construct; identical to SEQ ID NO: 500
  source 1..159: mol_type = other DNA

SEQ ID NO: 537 - DNA, 10 bp, synthetic construct
  source 1..10: mol_type = other DNA
ttttattttt

SEQ ID NO: 538 - DNA, 130 bp, synthetic construct
  source 1..130: mol_type = other DNA
ggcttcactgataaagtggagaaccgcttcaccgagtgaaggtgggctgcttgcatcagcctaatgtcgagaagtgctttcttcggaaagtaaccctcgaaacaaattcatttgaatgaaggaatgcaac

SEQ ID NO: 539 - DNA, 10 bp, synthetic construct
  source 1..10: mol_type = other DNA
ttttattttt

SEQ ID NO: 540 - DNA, 149 bp, synthetic construct; identical to SEQ ID NO: 512
  source 1..149: mol_type = other DNA

SEQ ID NO: 541 - DNA, 132 bp, synthetic construct; identical to SEQ ID NO: 513
  source 1..132: mol_type = other DNA

SEQ ID NO: 542 - DNA, 10 bp, synthetic construct
  source 1..10: mol_type = other DNA
ttttattttt

SEQ ID NO: 543 - DNA, 130 bp, synthetic construct; identical to SEQ ID NO: 538
  source 1..130: mol_type = other DNA

SEQ ID NO: 544 - DNA, 10 bp, synthetic construct
  source 1..10: mol_type = other DNA
ttttattttt

SEQ ID NO: 545 - DNA, 122 bp, synthetic construct
  source 1..122: mol_type = other DNA
ggcttcactgataaagtggagaaccgcttcacttagagtgaaggtgggctgcttgcatcagcctaatgtcgagaagtgctttcttcggaaagtaaccctcgaaacaaagaaaggaatgcaac

SEQ ID NO: 546 - DNA, 120 bp, synthetic construct
  source 1..120: mol_type = other DNA
ggcttcactgataaagtggagaaccgcttcaccgagtgaaggtgggctgcttgcatcagcctaatgtcgagaagtgctttcttcggaaagtaaccctcgaaacaaagaaaggaatgcaac

SEQ ID NO: 547 - DNA, 100 bp, synthetic construct
  source 1..100: mol_type = other DNA
accgcttcacttagagtgaaggtgggctgcttgcatcagcctaatgtcgagaagtgctttcttcggaaagtaaccctcgaaacaaagaaaggaatgcaac

SEQ ID NO: 548 - DNA, 98 bp, synthetic construct
  source 1..98: mol_type = other DNA
accgcttcaccgagtgaaggtgggctgcttgcatcagcctaatgtcgagaagtgctttcttcggaaagtaaccctcgaaacaaagaaaggaatgcaac

SEQ ID NO: 549 - DNA, 100 bp, synthetic construct; identical to SEQ ID NO: 547
  source 1..100: mol_type = other DNA

SEQ ID NO: 550 - DNA, 10 bp, synthetic construct
  source 1..10: mol_type = other DNA
ttttattttt

SEQ ID NO: 551 - DNA, 98 bp, synthetic construct; identical to SEQ ID NO: 548
  source 1..98: mol_type = other DNA

SEQ ID NO: 552 - DNA, 10 bp, synthetic construct
  source 1..10: mol_type = other DNA
ttttattttt

SEQ ID NO: 553 - DNA, 104 bp, synthetic construct
  source 1..104: mol_type = other DNA
accgcttcacgcttcggcagtgaaggtgggctgcttgcatcagcctaatgtcgagaagtgctttcttcggaaagtaaccctcgaaacaaagaaaggaatgcaac

SEQ ID NO: 554 - DNA, 104 bp, synthetic construct; identical to SEQ ID NO: 553
  source 1..104: mol_type = other DNA

SEQ ID NO: 555 - DNA, 103 bp, synthetic construct
  source 1..103: mol_type = other DNA
ccgcttcacgcttcggcagtgaaggtgggctgcttgcatcagcctaatgtcgagaagtgctttcttcggaaagtaaccctcgaaacaaagaaaggaatgcaac

SEQ ID NO: 556 - DNA, 103 bp, synthetic construct
  source 1..103: mol_type = other DNA
ccgcttcacgcttaggcagtgaaggtgggctgcttgcatcagcctaatgtcgagaagtgctttcttcggaaagtaaccctcgaaacaaagaaaggaatgcaac

SEQ ID NO: 557 - DNA, 103 bp, synthetic construct
  source 1..103: mol_type = other DNA
ccgcttcactcttaggaagtgaaggtgggctgcttgcatcagcctaatgtcgagaagtgctttcttcggaaagtaaccctcgaaacaaagaaaggaatgcaac

SEQ ID NO: 558 - DNA, 103 bp, synthetic construct
  source 1..103: mol_type = other DNA
ccgcttcacgtttagacagtgaaggtgggctgcttgcatcagcctaatgtcgagaagtgctttcttcggaaagtaaccctcgaaacaaagaaaggaatgcaac

SEQ ID NO: 559 - DNA, 104 bp, synthetic construct
  source 1..104: mol_type = other DNA
gccgcttcacgcttcggcagtgaaggtgggctgcttgcatcagcctaatgtcgagaagtgctttcttcggaaagtaaccctcgaaacaaagaaaggaatgcaac

SEQ ID NO: 560 - DNA, 105 bp, synthetic construct
  source 1..105: mol_type = other DNA
ggccgcttcacgcttcggcagtgaaggtgggctgcttgcatcagcctaatgtcgagaagtgctttcttcggaaagtaaccctcgaaacaaagaaaggaatgcaac

SEQ ID NO: 561 - DNA, 102 bp, synthetic construct
  source 1..102: mol_type = other DNA
cgcttcacgcttcggcagtgaaggtgggctgcttgcatcagcctaatgtcgagaagtgctttcttcggaaagtaaccctcgaaacaaagaaaggaatgcaac

SEQ ID NO: 562 - DNA, 101 bp, synthetic construct
  source 1..101: mol_type = other DNA
gcttcacgcttcggcagtgaaggtgggctgcttgcatcagcctaatgtcgagaagtgctttcttcggaaagtaaccctcgaaacaaagaaaggaatgcaac

SEQ ID NO: 563 - DNA, 102 bp, synthetic construct
  source 1..102: mol_type = other DNA
ggcttcacgcttcggcagtgaaggtgggctgcttgcatcagcctaatgtcgagaagtgctttcttcggaaagtaaccctcgaaacaaagaaaggaatgcaac

SEQ ID NO: 564 - DNA, 101 bp, synthetic construct; identical to SEQ ID NO: 562
  source 1..101: mol_type = other DNA

SEQ ID NO: 565 - DNA, 102 bp, synthetic construct; identical to SEQ ID NO: 563
  source 1..102: mol_type = other DNA

SEQ ID NO: 566 - DNA, 102 bp, synthetic construct
  source 1..102: mol_type = other DNA
ccgcttcacgcttcggcagtgaaggtgggctgcttgcatcagcctaatgtcgagaagtgctttcttcggaaagtaaccctcgaaacaaagaaggaatgcaac

SEQ ID NO: 567 - DNA, 101 bp, synthetic construct
  source 1..101: mol_type = other DNA
ccgcttcacgcttcggcagtgaaggtgggctgcttgcatcagcctaatgtcgagaagtgctttcttcggaaagtaaccctcgaaacaaagaggaatgcaac

SEQ ID NO: 568 - DNA, 100 bp, synthetic construct
  source 1..100: mol_type = other DNA
ccgcttcacgcttcggcagtgaaggtgggctgcttgcatcagcctaatgtcgagaagtgctttcttcggaaagtaaccctcgaaacaaagggaatgcaac

SEQ ID NO: 569 - DNA, 99 bp, synthetic construct
  source 1..99: mol_type = other DNA
ccgcttcacgcttcggcagtgaaggtgggctgcttgcatcagcctaatgtcgagaagtgctttcttcggaaagtaaccctcgaaacaaaggaatgcaac

SEQ ID NO: 570 - DNA, 98 bp, synthetic construct
  source 1..98: mol_type = other DNA
ccgcttcacgcttcggcagtgaaggtgggctgcttgcatcagcctaatgtcgagaagtgctttcttcggaaagtaaccctcgaaacaaggaatgcaac

SEQ ID NO: 571 - DNA, 97 bp, synthetic construct
  source 1..97: mol_type = other DNA
ccgcttcacgcttcggcagtgaaggtgggctgcttgcatcagcctaatgtcgagaagtgctttcttcggaaagtaaccctcgaaacaggaatgcaac

SEQ ID NO: 572 - DNA, 96 bp, synthetic construct
  source 1..96: mol_type = other DNA
ccgcttcacgcttcggcagtgaaggtgggctgcttgcatcagcctaatgtcgagaagtgctttcttcggaaagtaaccctcgaaacggaatgcaac

SEQ ID NO: 573 - DNA, 95 bp, synthetic construct
  source 1..95: mol_type = other DNA
ccgcttcacgcttcggcagtgaaggtgggctgcttgcatcagcctaatgtcgagaagtgctttcttcggaaagtaaccctcgaaaggaatgcaac

SEQ ID NO: 574 - DNA, 94 bp, synthetic construct
  source 1..94: mol_type = other DNA
ccgcttcacgcttcggcagtgaaggtgggctgcttgcatcagcctaatgtcgagaagtgctttcttcggaaagtaaccctcgaaggaatgcaac

SEQ ID NO: 575 - DNA, 93 bp, synthetic construct
  source 1..93: mol_type = other DNA
ccgcttcacgcttcggcagtgaaggtgggctgcttgcatcagcctaatgtcgagaagtgctttcttcggaaagtaaccctcgaggaatgcaac

SEQ ID NO: 576 - DNA, 97 bp, synthetic construct
  source 1..97: mol_type = other DNA
ccgcttcacgcttcggcagtgaaggtgggctgcttgcatcagcctaatgtcgagaagtgctttcttcggaaagtaaccctcgaaacaagaatgcaac

SEQ ID NO: 577 - DNA, 99 bp, synthetic construct; identical to SEQ ID NO: 569
  source 1..99: mol_type = other DNA

SEQ ID NO: 578 - DNA, 98 bp, synthetic construct
  source 1..98: mol_type = other DNA
ccgcttcacgcttcggcagtgaaggtgggctgcttgcatcagcctaatgtcgagaagtgctttcttcggaaagtaaccctcgaaacaaagaatgcaac

SEQ ID NO: 579 - DNA, 102 bp, synthetic construct
  source 1..102: mol_type = other DNA
ccgcttcacgcttcggcagtgaaggtgggctgcttgcatcagcctaatgtcgagaagtgctttcttcggaaagtaaccctcgaaacaaagaaagaatgcaac

SEQ ID NO: 580 - DNA, 100 bp, synthetic construct
  source 1..100: mol_type = other DNA
ccgcttcacgcttcggcagtgaaggtgggctgcttgcatcagcctaatgtcgagaagtgctttcttcggaaagtaaccctcgaaacaaagagaatgcaac

SEQ ID NO: 581 - DNA, 99 bp, synthetic construct
  source 1..99: mol_type = other DNA
ccgcttcacgcttcggcagtgaaggtgggctgcttgcatcagcctaatgtcgagaagtgctttcttcggaaagtaaccctcgaaacaaagaaatgcaac

SEQ ID NO: 582 - DNA, 96 bp, synthetic construct
  source 1..96: mol_type = other DNA
ccgcttcacgcttcggcagtgaaggtgggctgcttgcatcagcctaatgtcgagaagtgctttcttcggaaagtaaccctcgaaacagaatgcaac

SEQ ID NO: 583 - DNA, 95 bp, synthetic construct
  source 1..95: mol_type = other DNA
ccgcttcacgcttcggcagtgaaggtgggctgcttgcatcagcctaatgtcgagaagtgctttcttcggaaagtaaccctcgaaacaaatgcaac

SEQ ID NO: 584 - DNA, 103 bp, synthetic construct
  source 1..103: mol_type = other DNA
ccgcttcacgcttcggcagtgaaggtgggctgattgcatcagcctaatgtcgagaagtgctttcttcggaaagtaaccctcgaaacaaagaaaggaatgcaac

SEQ ID NO: 585 - DNA, 103 bp, synthetic construct
  source 1..103: mol_type = other DNA
ccgcttcacgcttcggcagtgaaggtaggctgcttgcatcagcctaatgtcgagaagtgctttcttcggaaagtaaccctcgaaacaaagaaaggaatgcaac

SEQ ID NO: 586 - DNA, 103 bp, synthetic construct
  source 1..103: mol_type = other DNA
ccgcttcacgcttcggcagtgaaggtgggctgcttgcatcagcccaatgtcgagaagtgctttcttcggaaagtaaccctcgaaacaaagaaaggaatgcaac

SEQ ID NO: 587 - DNA, 97 bp, synthetic construct
  source 1..97: mol_type = other DNA
ggcttcacgcttcggcagtgaaggtaggctgcttgcatcagcctaatgtcgagaagtgctttcttcggaaagtaaccctcgaaacaaggaatgcaac

SEQ ID NO: 588 - DNA, 97 bp, synthetic construct
  source 1..97: mol_type = other DNA
ggcttcacgcttcggcagtgaaggtgggctgcttgcatcagcccaatgtcgagaagtgctttcttcggaaagtaaccctcgaaacaaggaatgcaac

SEQ ID NO: 589 - DNA, 103 bp, synthetic construct
  source 1..103: mol_type = other DNA
ccgcttcactcttaggaagtgaaggtgggctgattgcatcagcctaatgtcgagaagtgctttcttcggaaagtaaccctcgaaacaaagaaaggaatgcaac

SEQ ID NO: 590 - DNA, 100 bp, synthetic construct
  source 1..100: mol_type = other DNA
ccgcttcacgcttcggcagtgaaggtgggctgattgcatcagcctaatgtcgagaagtgctttcttcggaaagtaaccctcgaaacaaagggaatgcaac

SEQ ID NO: 591 - DNA, 99 bp, synthetic construct
  source 1..99: mol_type = other DNA
ccgcttcacgcttcggcagtgaaggtgggctgattgcatcagcctaatgtcgagaagtgctttcttcggaaagtaaccctcgaaacaaaggaatgcaac

SEQ ID NO: 592 - DNA, 98 bp, synthetic construct
  source 1..98: mol_type = other DNA
ccgcttcacgcttcggcagtgaaggtgggctgattgcatcagcctaatgtcgagaagtgctttcttcggaaagtaaccctcgaaacaaggaatgcaac

SEQ ID NO: 593 - DNA, 97 bp, synthetic construct
  source 1..97: mol_type = other DNA
ccgcttcacgcttcggcagtgaaggtgggctgattgcatcagcctaatgtcgagaagtgctttcttcggaaagtaaccctcgaaacaagaatgcaac

SEQ ID NO: 594 - DNA, 99 bp, synthetic construct; identical to SEQ ID NO: 591
  source 1..99: mol_type = other DNA

SEQ ID NO: 595 - DNA, 98 bp, synthetic construct
  source 1..98: mol_type = other DNA
ccgcttcacgcttcggcagtgaaggtgggctgattgcatcagcctaatgtcgagaagtgctttcttcggaaagtaaccctcgaaacaaagaatgcaac

SEQ ID NO: 596 - DNA, 96 bp, synthetic construct
  source 1..96: mol_type = other DNA
ccgcttcacgcttcggcagtgaaggtgggctgattgcatcagcctaatgtcgagaagtgctttcttcggaaagtaaccctcgaaacagaatgcaac

SEQ ID NO: 597 - RNA, 30 nt, synthetic construct
  source 1..30: mol_type = other RNA
ccgcttcacgcttcggcagtgaaggtgggc

SEQ ID NO: 598 - RNA, 30 nt, synthetic construct
  source 1..30: mol_type = other RNA
ccgcttcactcttaggaagtgaaggtgggc

SEQ ID NO: 599 - RNA, 30 nt, synthetic construct
  source 1..30: mol_type = other RNA
gaaagtaaccctcgaaacaaagaatgcaac

SEQ ID NO: 600 - RNA, 30 nt, synthetic construct
  source 1..30: mol_type = other RNA
aagtaaccctcgaaacaaagggaatgcaac

SEQ ID NO: 601 - RNA, 30 nt, synthetic construct
  source 1..30: mol_type = other RNA
aaagtaaccctcgaaacaaaggaatgcaac

SEQ ID NO: 602-699 - skipped (98 entries)

SEQ ID NO: 700 - RNA, 23 nt, synthetic construct
  source 1..23: mol_type = other RNA
ggggcatttgagtcacctttcta

SEQ ID NO: 701 - RNA, 23 nt, synthetic construct
  source 1..23: mol_type = other RNA
tccagaggaaagtgagcagtggc

SEQ ID NO: 702 - RNA, 23 nt, synthetic construct
  source 1..23: mol_type = other RNA
attctgacgtggaccctcatggc

SEQ ID NO: 703 - RNA, 23 nt, synthetic construct
  source 1..23: mol_type = other RNA
agagaaggagcagtttgccaggt

SEQ ID NO: 704 - RNA, 23 nt, synthetic construct
  source 1..23: mol_type = other RNA
ttctcccagtctctcatctcgtc

SEQ ID NO: 705 - RNA, 23 nt, synthetic construct
  source 1..23: mol_type = other RNA
aaatagggaccatgggcctctgt

SEQ ID NO: 706 - RNA, 23 nt, synthetic construct
  source 1..23: mol_type = other RNA
gcacacgtggcagggctaaggca

SEQ ID NO: 707 - RNA, 23 nt, synthetic construct
  source 1..23: mol_type = other RNA
ccaccctgggcggtatgagccgg

SEQ ID NO: 708 - RNA, 23 nt, synthetic construct
  source 1..23: mol_type = other RNA
gataacattgacaggacaggaga

SEQ ID NO: 709 - RNA, 23 nt, synthetic construct
  source 1..23: mol_type = other RNA
tgcagggctggcactgaacactg

SEQ ID NO: 710 - RNA, 23 nt, synthetic construct
  source 1..23: mol_type = other RNA
gagcaatatgcgcttgtctaatt

SEQ ID NO: 711 - RNA, 23 nt, synthetic construct
  source 1..23: mol_type = other RNA
cagcaccaagcctctgtttccct

SEQ ID NO: 712 - RNA, 23 nt, synthetic construct
  source 1..23: mol_type = other RNA
cactctcaggggtccttccgcct

SEQ ID NO: 713 - RNA, 23 nt, synthetic construct
  source 1..23: mol_type = other RNA
tttgtggtccctgtgccccctca

SEQ ID NO: 714 - RNA, 23 nt, synthetic construct
  source 1..23: mol_type = other RNA
tggtccctgtgccccctcacccc

SEQ ID NO: 715 - RNA, 23 nt, synthetic construct
  source 1..23: mol_type = other RNA
agtcacctttctacaccagtgat

SEQ ID NO: 716 - RNA, 23 nt, synthetic construct
  source 1..23: mol_type = other RNA
ccaggtggctaggtggggaagat

SEQ ID NO: 717 - RNA, 23 nt, synthetic construct
  source 1..23: mol_type = other RNA
ggcctttgagagaaggagcagtt

SEQ ID NO: 718 - RNA, 23 nt, synthetic construct
  source 1..23: mol_type = other RNA
caggcccctgggaatacaaagat

SEQ ID NO: 719 - RNA, 23 nt, synthetic construct
  source 1..23: mol_type = other RNA
aggcgcttggcctggtgattatg

SEQ ID NO: 720 - RNA, 23 nt, synthetic construct
  source 1..23: mol_type = other RNA
taagggtctgggggggtcagaac

SEQ ID NO: 721 - RNA, 23 nt, synthetic construct
  source 1..23: mol_type = other RNA
gagggcttctttgccaccctggg

SEQ ID NO: 722 - RNA, 23 nt, synthetic construct
  source 1..23: mol_type = other RNA
tggggtgagggggcacagggacc

SEQ ID NO: 723 - RNA, 23 nt, synthetic construct
  source 1..23: mol_type = other RNA
aggccctcgtggctgattaggcc

SEQ ID NO: 724 - RNA, 23 nt, synthetic construct
  source 1..23: mol_type = other RNA
agtcctcctcagcccctgagctc

SEQ ID NO: 725 - RNA, 23 nt, synthetic construct
  source 1..23: mol_type = other RNA
agcctcagctcagttttcttgct

SEQ ID NO: 726 - RNA, 23 nt, synthetic construct
  source 1..23: mol_type = other RNA
atgaacgaacaagagagtgaatt

SEQ ID NO: 727 - RNA, 23 nt, synthetic construct
  source 1..23: mol_type = other RNA
ttgactgaatatatgagggcttt

SEQ ID NO: 728 - RNA, 23 nt, synthetic construct
  source 1..23: mol_type = other RNA
tattcccaggggcctgcaaataa

SEQ ID NO: 729 - RNA, 23 nt, synthetic construct
  source 1..23: mol_type = other RNA
tttgcaggcccctgggaatacaa

SEQ ID NO: 730 - RNA, 20 nt, synthetic construct
  source 1..20: mol_type = other RNA
cccagtcatctgcccccaag

SEQ ID NO: 731 - RNA, 20 nt, synthetic construct
  source 1..20: mol_type = other RNA
tggatcctgagtacctctcc

SEQ ID NO: 732 - RNA, 20 nt, synthetic construct
  source 1..20: mol_type = other RNA
attctccagtcattgggtct

SEQ ID NO: 733 - RNA, 20 nt, synthetic construct
  source 1..20: mol_type = other RNA
ctttagaataatgtcttgca

SEQ ID NO: 734 - RNA, 20 nt, synthetic construct
  source 1..20: mol_type = other RNA
tttagaataatgtcttgcat

SEQ ID NO: 735 - RNA, 20 nt, synthetic construct
  source 1..20: mol_type = other RNA
gaacgaacaagagagtgaat

SEQ ID NO: 736 - RNA, 20 nt, synthetic construct
  source 1..20: mol_type = other RNA
agggtctgggggggtcagaa

SEQ ID NO: 737 - RNA, 20 nt, synthetic construct
  source 1..20: mol_type = other RNA
ataatgtcttgcatttaaca

SEQ ID NO: 738 - RNA, 20 nt, synthetic construct
  source 1..20: mol_type = other RNA
aggaaaacagatggggtgct

SEQ ID NO: 739 - RNA, 20 nt, synthetic construct
  source 1..20: mol_type = other RNA
gatgtggccaggcagcaaca

SEQ ID NO: 740 - RNA, 20 nt, synthetic construct
  source 1..20: mol_type = other RNA
aatattgtccctttcactgt

SEQ ID NO: 741 - RNA, 20 nt, synthetic construct
  source 1..20: mol_type = other RNA
catgagggtccacgtcagaa

SEQ ID NO: 742 - RNA, 20 nt, synthetic construct
  source 1..20: mol_type = other RNA
cagacagatctgggaatcct

SEQ ID NO: 743 - RNA, 20 nt, synthetic construct
  source 1..20: mol_type = other RNA
aatcctgggtgggaagagag

SEQ ID NO: 744 - RNA, 20 nt, synthetic construct
  source 1..20: mol_type = other RNA
ccacagtaggtgctcaatac

SEQ ID NO: 745 - RNA, 20 nt, synthetic construct
  source 1..20: mol_type = other RNA
cccaaggctctgactacttt

SEQ ID NO: 746 - RNA, 20 nt, synthetic construct
  source 1..20: mol_type = other RNA
tactttctttctcacggtac

SEQ ID NO: 747 - RNA, 20 nt, synthetic construct
  source 1..20: mol_type = other RNA
atcggccctcgtggggccac

SEQ ID NO: 748 - RNA, 20 nt, synthetic construct
  source 1..20: mol_type = other RNA
gcctccagggccagcctccc

SEQ ID NO: 749 - RNA, 20 nt, synthetic construct
  source 1..20: mol_type = other RNA
cctcccctgctctggtagcc

SEQ ID NO: 750 - RNA, 20 nt, synthetic construct
  source 1..20: mol_type = other RNA
agccccctccatcctccctc

SEQ ID NO: 751 - RNA, 20 nt, synthetic construct
  source 1..20: mol_type = other RNA
caagccactgctcactttcc

SEQ ID NO: 752 - RNA, 20 nt, synthetic construct
  source 1..20: mol_type = other RNA
taaagccaggttccccggcc

SEQ ID NO: 753 - RNA, 20 nt, synthetic construct
  source 1..20: mol_type = other RNA
tcttcccacatttgagtcct

SEQ ID NO: 754 - RNA, 20 nt, synthetic construct
  source 1..20: mol_type = other RNA
cagggctgtttctttccatc

SEQ ID NO: 755 - RNA, 20 nt, synthetic construct
  source 1..20: mol_type = other RNA
accagaaacggaagctgcag

SEQ ID NO: 756 - RNA, 20 nt, synthetic construct
  source 1..20: mol_type = other RNA
gcggggattaatatgattat

SEQ ID NO: 757 - RNA, 20 nt, synthetic construct
  source 1..20: mol_type = other RNA
ggggtcagaacccagagtca

SEQ ID NO: 758 - RNA, 20 nt, synthetic construct
  source 1..20: mol_type = other RNA
gatcgtgctgggcttcccca

SEQ ID NO: 759 - RNA, 20 nt, synthetic construct
  source 1..20: mol_type = other RNA
tggatacttcgtcttcgggc

SEQ ID NO: 760 - RNA, 20 nt, synthetic construct
  source 1..20: mol_type = other RNA
agagtcccgggcttggcggt

SEQ ID NO: 761 - RNA, 20 nt, synthetic construct
  source 1..20: mol_type = other RNA
tcccttggagcagctgtgct

SEQ ID NO: 762 - RNA, 20 nt, synthetic construct
  source 1..20: mol_type = other RNA
ggcccaagctcagggtggga

SEQ ID NO: 763 - RNA, 20 nt, synthetic construct
  source 1..20: mol_type = other RNA
atcctctgcctcccctctca

SEQ ID NO: 764 - RNA, 20 nt, synthetic construct
  source 1..20: mol_type = other RNA
tcccctctcagcccctgtcc

SEQ ID NO: 765 - RNA, 20 nt, synthetic construct
  source 1..20: mol_type = other RNA
gcagttcctttttgctttag

SEQ ID NO: 766 - RNA, 20 nt, synthetic construct
  source 1..20: mol_type = other RNA
gagatgcaggaggagacgct

SEQ ID NO: 767 - RNA, 20 nt, synthetic construct
  source 1..20: mol_type = other RNA
agccttgccctgtctccccc

SEQ ID NO: 768 - RNA, 20 nt, synthetic construct
  source 1..20: mol_type = other RNA
cattgttgggtgtttgttgc

SEQ ID NO: 769 - RNA, 20 nt, synthetic construct
  source 1..20: mol_type = other RNA
cagaaggtgggtgtgccact

SEQ ID NO: 770 - RNA, 20 nt, synthetic construct
  source 1..20: mol_type = other RNA
gggtcagtcccagtttacaa

SEQ ID NO: 771 - RNA, 20 nt, synthetic construct
  source 1..20: mol_type = other RNA
ttccatcatttccttcttct

SEQ ID NO: 772 - RNA, 20 nt, synthetic construct
  source 1..20: mol_type = other RNA
caaaacattgcacattgctt

SEQ ID NO: 773 - RNA, 20 nt, synthetic construct
  source 1..20: mol_type = other RNA
tttccaccacctctgcattc

SEQ ID NO: 774 - RNA, 20 nt, synthetic construct
  source 1..20: mol_type = other RNA
ttccttcccaacaaggaact

SEQ ID NO: 775 - RNA, 20 nt, synthetic construct
  source 1..20: mol_type = other RNA
ccacattaggatgcattctt

SEQ ID NO: 776 - RNA, 20 nt, synthetic construct
  source 1..20: mol_type = other RNA
aaacacacacacacacacac

SEQ ID NO: 777 - RNA, 20 nt, synthetic construct
  source 1..20: mol_type = other RNA
tgtttgtggtccctgtgccc

SEQ ID NO: 778 - RNA, 20 nt, synthetic construct
  source 1..20: mol_type = other RNA
ccagtgatctgcccaagcca

SEQ ID NO: 779 - RNA, 20 nt, synthetic construct
  source 1..20: mol_type = other RNA
tgtccagaggacatagcaca

SEQ ID NO: 780 - RNA, 20 nt, synthetic construct
  source 1..20: mol_type = other RNA
tgcccttctccaatgcgacg

SEQ ID NO: 781 - RNA, 20 nt, synthetic construct
  source 1..20: mol_type = other RNA
tcaccgtccagcacaagaag

SEQ ID NO: 782 - RNA, 20 nt, synthetic construct
  source 1..20: mol_type = other RNA
cctctctgcatggatacttc

SEQ ID NO: 783 - RNA, 20 nt, synthetic construct
  source 1..20: mol_type = other RNA
ttcacagcaagaaaactgag

SEQ ID NO: 784 - RNA, 20 nt, synthetic construct
  source 1..20: mol_type = other RNA
tcgggccatgtttgcagcac

SEQ ID NO: 785 - RNA, 20 nt, synthetic construct
  source 1..20: mol_type = other RNA
gggggctcagcccggccagg

SEQ ID NO: 786 - RNA, 20 nt, synthetic construct
  source 1..20: mol_type = other RNA
ctcagcccggccagggaggg

SEQ ID NO: 787 - RNA, 20 nt, synthetic construct
  source 1..20: mol_type = other RNA
agtcttgacccaaggcatcc

SEQ ID NO: 788 - RNA, 20 nt, synthetic construct
  source 1..20: mol_type = other RNA
taatcaccaggccaagcgcc

SEQ ID NO: 789 - RNA, 20 nt, synthetic construct
  source 1..20: mol_type = other RNA
gcagggaagggggcactctt

SEQ ID NO: 790 - RNA, 20 nt, synthetic construct
  source 1..20: mol_type = other RNA
tcctgggtgggaagagagac

SEQ ID NO: 791 - RNA, 20 nt, synthetic construct
  source 1..20: mol_type = other RNA
ggaagagagacagtgagaga

SEQ ID NO: 792 - RNA, 20 nt, synthetic construct
  source 1..20: mol_type = other RNA
gagagacagtgagagagaga

SEQ ID NO: 793 - RNA, 20 nt, synthetic construct
  source 1..20: mol_type = other RNA
tggctgcgactgaactgtcc

SEQ ID NO: 794 - RNA, 20 nt, synthetic construct
  source 1..20: mol_type = other RNA
acagtaggtgctcaatacac

SEQ ID NO: 795 - RNA, 20 nt, synthetic construct
  source 1..20: mol_type = other RNA
gtgtcatcatgttgcttgga

SEQ ID NO: 796 - RNA, 20 nt, synthetic construct
  source 1..20: mol_type = other RNA
cacctggtacatggcatttg

SEQ ID NO: 797 - RNA, 20 nt, synthetic construct
  source 1..20: mol_type = other RNA
catttgagtcacctttctac

SEQ ID NO: 798 - RNA, 20 nt, synthetic construct
  source 1..20: mol_type = other RNA
gggctgtttctttccatctt

SEQ ID NO: 799 - RNA, 20 nt, synthetic construct
  source 1..20: mol_type = other RNA
cctgggccctaggctatgtg

SEQ ID NO: 800 - RNA, 20 nt, synthetic construct
  source 1..20: mol_type = other RNA
ctaggctatgtgtctggcac

SEQ ID NO: 801 - RNA, 20 nt, synthetic construct
  source 1..20: mol_type = other RNA
ctggatgactccagaggtaa

SEQ ID NO: 802 - RNA, 20 nt, synthetic construct
  source 1..20: mol_type = other RNA
aacgaacaggtaaggggctg

SEQ ID NO: 803 - RNA, 20 nt, synthetic construct
  source 1..20: mol_type = other RNA
aataaaccagaaagtctcta

SEQ ID NO: 804 - RNA, 20 nt, synthetic construct
  source 1..20: mol_type = other RNA
cttgggacagacaagtcatg

SEQ ID NO: 805 - RNA, 20 nt, synthetic construct
  source 1..20: mol_type = other RNA
agacaagtcatgcagaagtt

SEQ ID NO: 806 - RNA, 20 nt, synthetic construct
  source 1..20: mol_type = other RNA
ggtcagaacccagagtcatc

SEQ ID NO: 807 - RNA, 20 nt, synthetic construct
  source 1..20: mol_type = other RNA
ggagcagccacgggtcagcc

SEQ ID NO: 808 - RNA, 20 nt, synthetic construct
  source 1..20: mol_type = other RNA
cagccacgggtcagccacaa

SEQ ID NO: 809 - RNA, 20 nt, synthetic construct
  source 1..20: mol_type = other RNA
tccccatcaacttcctcacg

SEQ ID NO: 810 - RNA, 20 nt, synthetic construct
  source 1..20: mol_type = other RNA
gtatgagccgggtgtgggtg

SEQ ID NO: 811 - RNA, 20 nt, synthetic construct
  source 1..20: mol_type = other RNA
gggtgtgcaggagcccggga

SEQ ID NO: 812 - RNA, 20 nt, synthetic construct
  source 1..20: mol_type = other RNA
gtgcaggagcccgggagcat

SEQ ID NO: 813 - RNA, 20 nt, synthetic construct
  source 1..20: mol_type = other RNA
agtcccgggcttggcggtgg

SEQ ID NO: 814 - RNA, 20 nt, synthetic construct
  source 1..20: mol_type = other RNA
gtcacacagggacgggtgca

SEQ ID NO: 815 - RNA, 20 nt, synthetic construct
  source 1..20: mol_type = other RNA
ctgagggagagctgggcaag

SEQ ID NO: 816 - RNA, 20 nt, synthetic construct
  source 1..20: mol_type = other RNA
agccagacccctcctctctg

SEQ ID NO: 817 - RNA, 20 nt, synthetic construct
  source 1..20: mol_type = other RNA
cccaagctcagggtgggaag

SEQ ID NO: 818 - RNA, 20 nt, synthetic construct
  source 1..20: mol_type = other RNA
gtggattttccattctccag

SEQ ID NO: 819 - RNA, 20 nt, synthetic construct
  source 1..20: mol_type = other RNA
ttccctgtgctgggcaatgg

SEQ ID NO: 820 - RNA, 20 nt, synthetic construct
  source 1..20: mol_type = other RNA
atgggctcggtcccctctgg

SEQ ID NO: 821 - RNA, 20 nt, synthetic construct
  source 1..20: mol_type = other RNA
cggtcccctctggcatcctc

SEQ ID NO: 822 - RNA, 20 nt, synthetic construct
  source 1..20: mol_type = other RNA
gctgcagggataacagatcc

SEQ ID NO: 823 - RNA, 20 nt, synthetic construct
  source 1..20: mol_type = other RNA
acggggagagcttaccgcca

SEQ ID NO: 824 - RNA, 20 nt, synthetic construct
  source 1..20: mol_type = other RNA
tttgttgcattcaataatca

SEQ ID NO: 825 - RNA, 20 nt, synthetic construct
  source 1..20: mol_type = other RNA
tgccacttacgggtggttgt

SEQ ID NO: 826 - RNA, 20 nt, synthetic construct
  source 1..20: mol_type = other RNA
ggtgagcaggacagatgtct

SEQ ID NO: 827 - RNA, 20 nt, synthetic construct
  source 1..20: mol_type = other RNA
aaacattgcacattgcttca

SEQ ID NO: 828 - RNA, 20 nt, synthetic construct
  source 1..20: mol_type = other RNA
tccaccacctctgcattcct

SEQ ID NO: 829 - RNA, 20 nt, synthetic construct
  source 1..20: mol_type = other RNA
ccaggtgggctgcagggaag

SEQ ID NO: 830 - RNA, 20 nt, synthetic construct
  source 1..20: mol_type = other RNA
tccttcccctgaagcttcct

SEQ ID NO: 831 - RNA, 20 nt, synthetic construct
  source 1..20: mol_type = other RNA
gtggtcactgagcggccgca

SEQ ID NO: 832 - RNA, 20 nt, synthetic construct
  source 1..20: mol_type = other RNA
acggggagagggggacctgc

SEQ ID NO: 833 - RNA, 20 nt, synthetic construct
  source 1..20: mol_type = other RNA
cggccctcgtggggccacct

SEQ ID NO: 834 - RNA, 20 nt, synthetic construct
  source 1..20: mol_type = other RNA
cccctgaagggttctgcccc

SEQ ID NO: 835 - RNA, 20 nt, synthetic construct
  source 1..20: mol_type = other RNA
aagccaggttccccggccta

SEQ ID NO: 836 - RNA, 20 nt, synthetic construct
  source 1..20: mol_type = other RNA
gaatctgcttcttcccacat

SEQ ID NO: 837 - RNA, 20 nt, synthetic construct
  source 1..20: mol_type = other RNA
tcctcctgtcagaggagtgt

SEQ ID NO: 838 - RNA, 20 nt, synthetic construct
  source 1..20: mol_type = other RNA
actccagaggtaacttgtgg

SEQ ID NO: 839 - RNA, 20 nt, synthetic construct
  source 1..20: mol_type = other RNA
ctgagtacctctcctccctg

SEQ ID NO: 840 - RNA, 20 nt, synthetic construct
  source 1..20: mol_type = other RNA
cctgagtggctgagctcagg

SEQ ID NO: 841 - RNA, 20 nt, synthetic construct
  source 1..20: mol_type = other RNA
cttcgtcttcgggcccacag

SEQ ID NO: 842 - RNA, 20 nt, synthetic construct
  source 1..20: mol_type = other RNA
gcttctttgccaccctgggc

SEQ ID NO: 843 - RNA, 20 nt, synthetic construct
  source 1..20: mol_type = other RNA
ggtctgggagagtcccgggc

SEQ ID NO: 844 - RNA, 20 nt, synthetic construct
  source 1..20: mol_type = other RNA
aatatgcgcttgtctaattt

SEQ ID NO: 845 - RNA, 20 nt, synthetic construct
  source 1..20: mol_type = other RNA
cccgcatctatctcgggcca

SEQ ID NO: 846 - RNA, 20 nt, synthetic construct
  source 1..20: mol_type = other RNA
agctgtgctgagtcagaccc

SEQ ID NO: 847 - RNA, 20 nt, synthetic construct
  source 1..20: mol_type = other RNA
ttccattctccagtcattgg

SEQ ID NO: 848 - RNA, 20 nt, synthetic construct
  source 1..20: mol_type = other RNA
ttcatggggtggtgagcagg

SEQ ID NO: 849 - RNA, 20 nt, synthetic construct
  source 1..20: mol_type = other RNA
caggcagggagacgggcaca

SEQ ID NO: 850 - RNA, 20 nt, synthetic construct
  source 1..20: mol_type = other RNA
acgggcacaaaacacaaata

SEQ ID NO: 851 - RNA, 20 nt, synthetic construct
  source 1..20: mol_type = other RNA
cgggcacaaaacacaaataa

SEQ ID NO: 852 - RNA, 20 nt, synthetic construct
  source 1..20: mol_type = other RNA
gctgtcagaagcactatgca

SEQ ID NO: 853 - RNA, 20 nt, synthetic construct
  source 1..20: mol_type = other RNA
ctgtcagaagcactatgcaa

SEQ ID NO: 854 - RNA, 20 nt, synthetic construct
  source 1..20: mol_type = other RNA
ctgtgccccctcaccccaca

SEQ ID NO: 855 - RNA, 20 nt, synthetic construct
  source 1..20: mol_type = other RNA
tgtgccccctcaccccacaa

SEQ ID NO: 856 - RNA, 20 nt, synthetic construct
  source 1..20: mol_type = other RNA
tgtctaatttcacagcaaga

SEQ ID NO: 857 - RNA, 20 nt, synthetic construct
  source 1..20: mol_type = other RNA
atgtcttgcatttaacagga

SEQ ID NO: 858 - RNA, 20 nt, synthetic construct
  source 1..20: mol_type = other RNA
atcccacttaacagagagga

SEQ ID NO: 859 - RNA, 20 nt, synthetic construct
  source 1..20: mol_type = other RNA
ccttcttcttcctctgggca

SEQ ID NO: 860 - RNA, 20 nt, synthetic construct
  source 1..20: mol_type = other RNA
cacacacacacacacacaca

SEQ ID NO: 861 - RNA, 20 nt, synthetic construct
  source 1..20: mol_type = other RNA
gacgggcacaaaacacaaat

SEQ ID NO: 862 - RNA, 20 nt, synthetic construct
  source 1..20: mol_type = other RNA
atcaccaggccaagcgcctt

SEQ ID NO: 863 - RNA, 20 nt, synthetic construct
  source 1..20: mol_type = other RNA
ctgctcactttcctctggat

SEQ ID NO: 864 - RNA, 20 nt, synthetic construct
  source 1..20: mol_type = other RNA
attcccaggggcctgcaaat

SEQ ID NO: 865 - RNA, 20 nt, synthetic construct
  source 1..20: mol_type = other RNA
agatgagagactgggagaat

SEQ ID NO: 866 - RNA, 20 nt, synthetic construct
  source 1..20: mol_type = other RNA
ttaggatgcattcttctgct

SEQ ID NO: 867 - RNA, 20 nt, synthetic construct
  source 1..20: mol_type = other RNA
taaaaagcttccatgctgtc

SEQ ID NO: 868 - RNA, 20 nt, synthetic construct
  source 1..20: mol_type = other RNA
tggccatgagggtccacgtc

SEQ ID NO: 869 - RNA, 20 nt, synthetic construct
  source 1..20: mol_type = other RNA
aagggggcactcttctgagc

SEQ ID NO: 870 - RNA, 20 nt, synthetic construct
  source 1..20: mol_type = other RNA
gggcactcttctgagcagac

SEQ ID NO: 871 - RNA, 20 nt, synthetic construct
  source 1..20: mol_type = other RNA
agctcaagctccagcttctc

SEQ ID NO: 872 - RNA, 20 nt, synthetic construct
  source 1..20: mol_type = other RNA
gcacttgcacaggccgcccc

SEQ ID NO: 873 - RNA, 20 nt, synthetic construct
  source 1..20: mol_type = other RNA
ccagggccagcctccccttc

SEQ ID NO: 874 - RNA, 20 nt, synthetic construct
  source 1..20: mol_type = other RNA
attacaactgcccccagccc

SEQ ID NO: 875 - RNA, 20 nt, synthetic construct
  source 1..20: mol_type = other RNA
aggcccaaacatggcctccc

SEQ ID NO: 876 - RNA, 20 nt, synthetic construct
  source 1..20: mol_type = other RNA
taggctatgtgtctggcacc

SEQ ID NO: 877 - RNA, 20 nt, synthetic construct
  source 1..20: mol_type = other RNA
ctcatggagctcctcctgtc

SEQ ID NO: 878 - RNA, 20 nt, synthetic construct
  source 1..20: mol_type = other RNA
tgtggggactggatgactcc

SEQ ID NO: 879 - RNA, 20 nt, synthetic construct
  source 1..20: mol_type = other RNA
agagactgggagaataaacc

SEQ ID NO: 880 - RNA, 20 nt, synthetic construct
  source 1..20: mol_type = other RNA
cagaaagtctctagctgtcc

SEQ ID NO: 881 - RNA, 20 nt, synthetic construct
  source 1..20: mol_type = other RNA
ctgtccagaggacatagcac

SEQ ID NO: 882 - RNA, 20 nt, synthetic construct
  source 1..20: mol_type = other RNA
tatttcaaacccaggccacc

SEQ ID NO: 883 - RNA, 20 nt, synthetic construct
  source 1..20: mol_type = other RNA
ctgagctgggaccttgggac

SEQ ID NO: 884 - RNA, 20 nt, synthetic construct
  source 1..20: mol_type = other RNA
ttgggacagacaagtcatgc

SEQ ID NO: 885 - RNA, 20 nt, synthetic construct
  source 1..20: mol_type = other RNA
atgaacacccccaatctccc

SEQ ID NO: 886 - RNA, 20 nt, synthetic construct
  source 1..20: mol_type = other RNA
tataagggtctgggggggtc

SEQ ID NO: 887 - RNA, 20 nt, synthetic construct
  source 1..20: mol_type = other RNA
gtctgggggggtcagaaccc

SEQ ID NO: 888 - RNA, 20 nt, synthetic construct
  source 1..20: mol_type = other RNA
gccacagccatgaatggcac

SEQ ID NO: 889 - RNA, 20 nt, synthetic construct
  source 1..20: mol_type = other RNA
cgtcacacagggacgggtgc

SEQ ID NO: 890 - RNA, 20 nt, synthetic construct
  source 1..20: mol_type = other RNA
tggagcagctgtgctgagtc

SEQ ID NO: 891 - RNA, 20 nt, synthetic construct
  source 1..20: mol_type = other RNA
gagggagagctgggcaagcc

SEQ ID NO: 892 - RNA, 20 nt, synthetic construct
  source 1..20: mol_type = other RNA
cttgcatttaacaggaaaac

SEQ ID NO: 893 - RNA, 20 nt, synthetic construct
  source 1..20: mol_type = other RNA
tggggtgctgcagggataac

SEQ ID NO: 894 - RNA, 20 nt, synthetic construct
  source 1..20: mol_type = other RNA
gataacagatcccacttaac

SEQ ID NO: 895 - RNA, 20 nt, synthetic construct
  source 1..20: mol_type = other RNA
ttgttgcattcaataatcac

SEQ ID NO: 896 - RNA, 20 nt, synthetic construct
  source 1..20: mol_type = other RNA
cagatcactcagttctggcc

SEQ ID NO: 897 - RNA, 20 nt, synthetic construct
  source 1..20: mol_type = other RNA
catggggtggtgagcaggac

SEQ ID NO: 898 - RNA, 20 nt, synthetic construct
  source 1..20: mol_type = other RNA
cacgtgtgccaaacgctgtt

SEQ ID NO: 899 - RNA, 20 nt, synthetic construct
  source 1..20: mol_type = other RNA
gtgtcaccttggcccctctt

SEQ ID NO: 900 - RNA, 20 nt, synthetic construct
  source 1..20: mol_type = other RNA
aagcagttcctttttgcttt

SEQ ID NO: 901 - RNA, 20 nt, synthetic construct
  source 1..20: mol_type = other RNA
gagatgcaggaggagacgct

SEQ ID NO: 902 - RNA, 20 nt, synthetic construct
  source 1..20: mol_type = other RNA
agttgtgggtcagtcttgac

SEQ ID NO: 903 - RNA, 20 nt, synthetic construct
  source 1..20: mol_type = other RNA
gccttaaactacgagaggcc

SEQ ID NO: 904 - RNA, 20 nt, synthetic construct
  source 1..20: mol_type = other RNA
aaactacgagaggccccatc

SEQ ID NO: 905 - RNA, 20 nt, synthetic construct
  source 1..20: mol_type = other RNA
tgtgccaaacgctgttagac

SEQ ID NO: 906 - RNA, 20 nt, synthetic construct
  source 1..20: mol_type = other RNA
gctgttagacccaacaccac

SEQ ID NO: 907 - RNA, 20 nt, synthetic construct
  source 1..20: mol_type = other RNA
gccaggtagggggctggagc

SEQ ID NO: 908 - RNA, 20 nt, synthetic construct
  source 1..20: mol_type = other RNA
gagagattaagggatatttc

SEQ ID NO: 909 - RNA, 20 nt, synthetic construct
  source 1..20: mol_type = other RNA
ggtccctgtgccccctcacc

SEQ ID NO: 910 - RNA, 20 nt, synthetic construct
  source 1..20: mol_type = other RNA
ttcgtgtaccctgtgtgtcc

SEQ ID NO: 911 - RNA, 20 nt, synthetic construct
  source 1..20: mol_type = other RNA
cagcacttgcacaggccgcc

SEQ ID NO: 912 - RNA, 20 nt, synthetic construct
  source 1..20: mol_type = other RNA
cacaggccgccccagacacc

SEQ ID NO: 913 - RNA, 20 nt, synthetic construct
  source 1..20: mol_type = other RNA
gtgttggcctccattttccc

SEQ ID NO: 914 - RNA, 20 nt, synthetic construct
  source 1..20: mol_type = other RNA
tttcccccagtcatctgccc

SEQ ID NO: 915 - RNA, 20 nt, synthetic construct
  source 1..20: mol_type = other RNA
cgtggctgattaggcctccc

SEQ ID NO: 916 - RNA, 20 nt, synthetic construct
  source 1..20: mol_type = other RNA
tttctacaccagtgatctgc

SEQ ID NO: 917 - RNA, 20 nt, synthetic construct
  source 1..20: mol_type = other RNA
cccggcctagcgttcaagac

SEQ ID NO: 918 - RNA, 20 nt, synthetic construct
  source 1..20: mol_type = other RNA
aagacccattacaactgccc

SEQ ID NO: 919 - RNA, 20 nt, synthetic construct
  source 1..20: mol_type = other RNA
ccattacaactgcccccagc

SEQ ID NO: 920 - RNA, 20 nt, synthetic construct
  source 1..20: mol_type = other RNA
gcccccagcccagatcttcc

SEQ ID NO: 921 - RNA, 20 nt, synthetic construct
  source 1..20: mol_type = other RNA
ctgctccttctctcaaaggc

SEQ ID NO: 922 - RNA, 20 nt, synthetic construct
  source 1..20: mol_type = other RNA
aaaggcccaaacatggcctc

SEQ ID NO: 923 - RNA, 20 nt, synthetic construct
  source 1..20: mol_type = other RNA
ggcctcccagactgcaaccc

SEQ ID NO: 924 - RNA, 20 nt, synthetic construct
  source 1..20: mol_type = other RNA
tggacggaatctgcttcttc

SEQ ID NO: 925 - RNA, 20 nt, synthetic construct
  source 1..20: mol_type = other RNA
ttctttccatctttgtattc

SEQ ID NO: 926 - RNA, 20 nt, synthetic construct
  source 1..20: mol_type = other RNA
agaggacatagcacagaggc

SEQ ID NO: 927 - RNA, 20 nt, synthetic construct
  source 1..20: mol_type = other RNA
ccatggtccctatttcaaac

SEQ ID NO: 928 - RNA, 20 nt, synthetic construct
  source 1..20: mol_type = other RNA
taatatgattatgaacaccc

SEQ ID NO: 929 - RNA, 20 nt, synthetic construct
  source 1..20: mol_type = other RNA
ttatgaacacccccaatctc

SEQ ID NO: 930 - RNA, 20 nt, synthetic construct
  source 1..20: mol_type = other RNA
gggtctgggggggtcagaac

SEQ ID NO: 931 - RNA, 20 nt, synthetic construct
  source 1..20: mol_type = other RNA
tacgcagccccttcgagtac

SEQ ID NO: 932 - RNA, 20 nt, synthetic construct
  source 1..20: mol_type = other RNA
gctgatcgtgctgggcttcc

SEQ ID NO: 933 - RNA, 20 nt, synthetic construct
  source 1..20: mol_type = other RNA
tggatacttcgtcttcgggc

SEQ ID NO: 934 - RNA, 20 nt, synthetic construct
  source 1..20: mol_type = other RNA
gcagctgtgctgagtcagac

SEQ ID NO: 935 - RNA, 20 nt, synthetic construct
  source 1..20: mol_type = other RNA
gacccctcctctctgggggc

SEQ ID NO: 936 - RNA, 20 nt, synthetic construct
  source 1..20: mol_type = other RNA
tgctgcagggataacagatc

SEQ ID NO: 937 - RNA, 20 nt, synthetic construct
  source 1..20: mol_type = other RNA
ccagccttgccctgtctccc

SEQ ID NO: 938 - RNA, 20 nt, synthetic construct
  source 1..20: mol_type = other RNA
tccaggctgctgcctcggtc

SEQ ID NO: 939 - RNA, 20 nt, synthetic construct
  source 1..20: mol_type = other RNA
tgttctctgcagggtcagtc

SEQ ID NO: 940 - RNA, 20 nt, synthetic construct
  source 1..20: mol_type = other RNA
ctttcactgttaggaatgtc

SEQ ID NO: 941 - RNA, 20 nt, synthetic construct
  source 1..20: mol_type = other RNA
atggctcctaggagaggccc

SEQ ID NO: 942 - RNA, 20 nt, synthetic construct
  source 1..20: mol_type = other RNA
caccacctctgcattccttc

SEQ ID NO: 943 - RNA, 20 nt, synthetic construct
  source 1..20: mol_type = other RNA
tcccaacaaggaactctgcc

SEQ ID NO: 944 - RNA, 20 nt, synthetic construct
  source 1..20: mol_type = other RNA
aaaacacaaataaaaagctt

SEQ ID NO: 945 - RNA, 20 nt, synthetic construct
  source 1..20: mol_type = other RNA
tcagtcttgacccaaggcat

SEQ ID NO: 946 - RNA, 20 nt, synthetic construct
  source 1..20: mol_type = other RNA
ctccttctggccatgagggt

SEQ ID NO: 947 - RNA, 20 nt, synthetic construct
  source 1..20: mol_type = other RNA
ttccgttctcagctcaagct

SEQ ID NO: 948 - RNA, 20 nt, synthetic construct
  source 1..20: mol_type = other RNA
ggacctgccagtgttggcct

SEQ ID NO: 949 - RNA, 20 nt, synthetic construct
  source 1..20: mol_type = other RNA
cagtgccctgtctgctgcct

SEQ ID NO: 950 - RNA, 20 nt, synthetic construct
  source 1..20: mol_type = other RNA
ccctgctctggtagccccct

SEQ ID NO: 951 - RNA, 20 nt, synthetic construct
  source 1..20: mol_type = other RNA
cccctccatcctccctccct

SEQ ID NO: 952 - RNA, 20 nt, synthetic construct
  source 1..20: mol_type = other RNA
ccatcctccctccctccact

SEQ ID NO: 953 - RNA, 20 nt, synthetic construct
  source 1..20: mol_type = other RNA
caggcagtcaggccctgtct

SEQ ID NO: 954 - RNA, 20 nt, synthetic construct
  source 1..20: mol_type = other RNA
ctgggcagggctgtttcttt

SEQ ID NO: 955 - RNA, 20 nt, synthetic construct
  source 1..20: mol_type = other RNA
aacgaacaagagagtgaatt

SEQ ID NO: 956 - RNA, 20 nt, synthetic construct
  source 1..20: mol_type = other RNA
caagagagtgaattccaatt

SEQ ID NO: 957 - RNA, 20 nt, synthetic construct
  source 1..20: mol_type = other RNA
agtgtggggactggatgact

SEQ ID NO: 958 - RNA, 20 nt, synthetic construct
  source 1..20: mol_type = other RNA
accagaaagtctctagctgt

SEQ ID NO: 959 - RNA, 20 nt, synthetic construct
  source 1..20: mol_type = other RNA
gggtcagaacccagagtcat

SEQ ID NO: 960 - RNA, 20 nt, synthetic construct
  source 1..20: mol_type = other RNA
taacttctacgtgcccttct

SEQ ID NO: 961 - RNA, 20 nt, synthetic construct
  source 1..20: mol_type = other RNA
ggctgagccatggcagttct

SEQ ID NO: 962 - RNA, 20 nt, synthetic construct
  source 1..20: mol_type = other RNA
ctcacgctctacgtcaccgt

SEQ ID NO: 963 - RNA, 20 nt, synthetic construct
  source 1..20: mol_type = other RNA
tcctgtcctgtcaatgttat

SEQ ID NO: 964 - RNA, 20 nt, synthetic construct
  source 1..20: mol_type = other RNA
cagggtgggaagtggatttt

SEQ ID NO: 965 - RNA, 20 nt, synthetic construct
  source 1..20: mol_type = other RNA
ggaagtggattttccattct

SEQ ID NO: 966 - RNA, 20 nt, synthetic construct
  source 1..20: mol_type = other RNA
ccctgtcctcaggtgcccct

SEQ ID NO: 967 - RNA, 20 nt, synthetic construct
  source 1..20: mol_type = other RNA
tccagcctccctgccgcgtt

SEQ ID NO: 968 - RNA, 20 nt, synthetic construct
  source 1..20: mol_type = other RNA
ctaggtctcctggctgtgat

SEQ ID NO: 969 - RNA, 20 nt, synthetic construct
  source 1..20: mol_type = other RNA
ttgccctgtctcccccatgt

SEQ ID NO: 970 - RNA, 20 nt, synthetic construct
  source 1..20: mol_type = other RNA
gcaggacagatgtctgaatt

SEQ ID NO: 971 - RNA, 20 nt, synthetic construct
  source 1..20: mol_type = other RNA
ggactgccaattctgggttt

SEQ ID NO: 972 - RNA, 20 nt, synthetic construct
  source 1..20: mol_type = other RNA
tcgtgtcccttatgggccca

SEQ ID NO: 973 - RNA, 20 nt, synthetic construct
  source 1..20: mol_type = other RNA
cagagcgctaagcaaataac

SEQ ID NO: 974 - RNA, 20 nt, synthetic construct
  source 1..20: mol_type = other RNA
ttcgtgtcccttatgggccc

SEQ ID NO: 975 - RNA, 20 nt, synthetic construct
  source 1..20: mol_type = other RNA
caaaactccctaccgggttc

SEQ ID NO: 976 - RNA, 20 nt, synthetic construct
  source 1..20: mol_type = other RNA
gattcgtgtcccttatgggc

SEQ ID NO: 977 - DNA, 1000 bp, Homo sapiens
  source 1..1000: mol_type = genomic DNA
ccttcagactggagtcccctgaagggttctgcccctcccctgctctggtagccccctccatcctccctccctccactccatctttgggggcatttgagtcacctttctacaccagtgatctgcccaagccactgctcactttcctctggataaagccaggttccccggcctagcgttcaagacccattacaactgcccccagcccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaaccc

SEQ ID NO: 978 - DNA, 500 bp, Homo sapiens
  source 1..500: mol_type = genomic DNA
tatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaaccc

SEQ ID NO: 979 - DNA, 1000 bp, Homo sapiens
  source 1..1000: mol_type = genomic DNA
agagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcatggaggggtctgggagagtcccgggcttggcggtggtggctgagaggccttctcccttctcctgtcctgtcaatgttatccaaagccctcatatattcagtcaacaaacaccattcatggtgatagccgggctgctgtttgtgcagggctggcactgaacactgccttgatcttatttggagcaatatgcgcttgtctaatttcacagcaagaaaactgagctgaggctcaaagaagtcaagcgccctgctggggcgtcacacagggacgggtgcagagttgagttggaagcccgcatctatctcgggccatgtttgcagcaccaagcctctgtttcccttggagcagctgtgctgagtcagacccaggctgggcactgagggagagctgggcaagccagacccctcctctctgggggcccaagctcagggtgggaagtggattttccattctccagtcattgggtcttccctgtgctgggcaatgggctcggtcccctct

SEQ ID NO: 980 - DNA, 500 bp, Homo sapiens
  source 1..500: mol_type = genomic DNA
agagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcat

SEQ ID NO: 981 - DNA, 1000 bp, Homo sapiens
  source 1..1000: mol_type = genomic DNA
gggttctgacccccccagacccttataaagtgacctccccctcctaagctcctggctgaatcagcatctgggagattgggggtgttcataatcatattaatccccgctgcagaaactgagggcctaattggcttctaagaggggccaaggtgacactaggaggaagcctgaggtcagggaggagaggtactcaggatccaggaaaagggaggagaaggtcccctaacttctgcatgacttgtctgtcccaaggtcccagctcagtctggtggcctgggtttgaaatagggaccatgggcctctgtgctatgtcctctggacagctagagactttctggtttattctcccagtctctcatctcgtcacacagccccttacctgttcgttcccccacaagttacctctggagtcatccagtccccacactcctctgacaggaggagctccatgagggcaggggctgcaacctgcagcttccgtttctggtgccagacacatagcctagggcccaggagcccaatccttgttgcatggaattggaattcactctcttgttcgttcattaaacatttatttgcaggcccctgggaatacaaagatggaaagaaacagccctgcccagaggagctcaggggctgaggaggactcaaatgtgggaagaagcagattccgtccagggtggctgtgaggttgtggagacagggcctgactgcctgggggttgcagtctgggaggccatgtttgggcctttgagagaaggagcagtttgccaggtggctaggtggggaagatctgggctgggggcagttgtaatgggtcttgaacgctaggccggggaacctggctttatccagaggaaagtgagcagtggcttgggcagatcactggtgtagaaaggtgactcaaatgcccccaaagatggagtggagggagggaggatggagggggctaccagagcaggggaggggcagaacccttcaggggactccagtctgaagg

SEQ ID NO: 982 - DNA, 500 bp, Homo sapiens
  source 1..500: mol_type = genomic DNA
gggttctgacccccccagacccttataaagtgacctccccctcctaagctcctggctgaatcagcatctgggagattgggggtgttcataatcatattaatccccgctgcagaaactgagggcctaattggcttctaagaggggccaaggtgacactaggaggaagcctgaggtcagggaggagaggtactcaggatccaggaaaagggaggagaaggtcccctaacttctgcatgacttgtctgtcccaaggtcccagctcagtctggtggcctgggtttgaaatagggaccatgggcctctgtgctatgtcctctggacagctagagactttctggtttattctcccagtctctcatctcgtcacacagccccttacctgttcgttcccccacaagttacctctggagtcatccagtccccacactcctctgacaggaggagctccatgagggcaggggctgcaacctgcagcttccgtttctggtgccagacacata

SEQ ID NO: 983 - DNA, 1000 bp, Homo sapiens
  source 1..1000: mol_type = genomic DNA
agaggggaccgagcccattgcccagcacagggaagacccaatgactggagaatggaaaatccacttcccaccctgagcttgggcccccagagaggaggggtctggcttgcccagctctccctcagtgcccagcctgggtctgactcagcacagctgctccaagggaaacagaggcttggtgctgcaaacatggcccgagatagatgcgggcttccaactcaactctgcacccgtccctgtgtgacgccccagcagggcgcttgacttctttgagcctcagctcagttttcttgctgtgaaattagacaagcgcatattgctccaaataagatcaaggcagtgttcagtgccagccctgcacaaacagcagcccggctatcaccatgaatggtgtttgttgactgaatatatgagggctttggataacattgacaggacaggagaagggagaaggcctctcagccaccaccgccaagcccgggactctcccagacccctccatgctcccgggctcctgcacaccccacccacacccggctcataccgcccagggtggcaaagaagccctccaaattgcatcctgtgggcccgaagacgaagtatccatgcagagaggtgtagagggtgctggtgaagccacctaggaccatgaagaggtcagccacggctaggttgagcaggatgtagttgagaggcgtgcgcagcttcttgtgctggacggtgacgtagagcgtgaggaagttgatggggaagcccagcacgatcagcagaaacatgtaggcggccagcatggagaactgccatggctcagccaggtagtactgtgggtactcgaaggggctgcgtaccacacccgtcgcattggagaagggcacgtagaagttagggccttctgtgccattcatggctgtggcccttgtggctgacccgtggctgctcccacccaagaatgctgcgaaggcctgagctcagccactcagggctccagctggatgactct

SEQ ID NO: 984 - DNA, 500 bp, Homo sapiens
  source 1..500: mol_type = genomic DNA
atgctcccgggctcctgcacaccccacccacacccggctcataccgcccagggtggcaaagaagccctccaaattgcatcctgtgggcccgaagacgaagtatccatgcagagaggtgtagagggtgctggtgaagccacctaggaccatgaagaggtcagccacggctaggttgagcaggatgtagttgagaggcgtgcgcagcttcttgtgctggacggtgacgtagagcgtgaggaagttgatggggaagcccagcacgatcagcagaaacatgtaggcggccagcatggagaactgccatggctcagccaggtagtactgtgggtactcgaaggggctgcgtaccacacccgtcgcattggagaagggcacgtagaagttagggccttctgtgccattcatggctgtggcccttgtggctgacccgtggctgctcccacccaagaatgctgcgaaggcctgagctcagccactcagggctccagctggatgactct